(19) United States
(12) Patent Application Publication
(10) Pub. No.: US 2025/0079000 A1
(43) Pub. Date: Mar. 6, 2025

(54) METHODS AND MARKERS FOR DIAGNOSIS OF PARKINSON'S DISEASE

(71) Applicant: Berg LLC, Framingham, MA (US)

(72) Inventors: Michael Andrew Kiebish, Millis, MA (US); Niven Rajin Narain, Cambridge, MA (US); Paula Patricia Narain, Cambridge, MA (US); Rangaprasad Sarangarajan, Boylston, MA (US)

(21) Appl. No.: 18/290,575

(22) PCT Filed: May 13, 2022
(86) PCT No.: PCT/US2022/029311

§ 371 Date: Nov. 14, 2023

Related U.S. Application Data
(60) Provisional application No. 63/188,677, filed on May 14, 2021.

(51) Int. Cl. G16H 50/20; C12Q 1/6883

(52) U.S. Cl. CPC G16H 50/20; C12Q 1/6883; C12Q 2600/158

(57) ABSTRACT

Methods for diagnosing the presence of Parkinson's disease in a subject are provided, such methods including the detection of levels of markers diagnostic of Parkinson's disease, including proteins, nucleic acids, and lipids, and optionally, determining performance in a clinical test such as an anxiety test, a sleep test, a smell test or any combination thereof. The invention also provides methods of treating Parkinson's disease by modulating the level or activity of the marker proteins, nucleic acids and lipids. Compositions in the form of kits and panels of reagents for detecting the markers of the invention, and optionally determining performance in a clinical test such as an anxiety test, a sleep test, a smell test or any combination thereof, are also provided.

### RELATED APPLICATIONS

This application claims priority to U.S. Provisional Application No. 63/188,677, filed on May 14, 2021, the entire contents of which are hereby incorporated herein by reference.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### BACKGROUND

Parkinson's disease (PD) is a degenerative disorder of the central nervous system. Because there is no definitive test for the diagnosis of PD, the disease must be diagnosed based on clinical criteria. Tremor at rest, slowness of movement (bradykinesia), rigidity, and postural instability are generally considered the cardinal signs of PD. The motor symptoms of Parkinson's disease result from the death of dopamine-generating cells in the substantia nigra, a region of the midbrain. The cause of this cell death is unknown. Early in the course of the disease, the most obvious symptoms are movement-related; these include shaking, rigidity, slowness of movement and difficulty with walking and gait. Later in the progression of PD, thinking and behavioral problems may arise, with dementia commonly occurring in the advanced stages of the disease. Depression is the most common psychiatric symptom. Other symptoms include sensory, sleep and emotional problems. Parkinson's disease is more common in older people, with most cases occurring after the age of 50.

Parkinson's disease is often defined as a parkinsonian syndrome that is idiopathic (having no known cause), although some atypical cases have a genetic origin. For example, variants of leucine-rich repeat kinase 2 (LRRK2), encoded by the PARK8 gene, are associated with an increased risk of Parkinson's disease type 8. Paisán-Ruíz et al., 2004, Neuron 44 (4): 595-600. Mutations in the glucocerebrosidase (GBA) gene are associated with susceptibility to familial Parkinson disease susceptibility and earlier onset of the disease. Nichols, et al., 2009, Neurology 72 (4): 310-316. In addition, mutations in the α-synuclein (SNCA) gene cause a rare dominant form of PD in familial and sporadic cases, and loss-of-function mutations in Parkin, PINK1, DJ-1 and ATP13A2 cause autosomal recessive parkinsonism with early-onset. Lesage et al., 2009, Human Molecular Genetics 18, Review Issue 1, R48-R59.

Many risk and protective factors for PD have been investigated. The clearest evidence is for an increased risk of PD in people exposed to certain pesticides, and a reduced risk in tobacco smokers. The pathology of the disease is characterized by the accumulation of the protein alpha-synuclein in inclusion bodies in neurons known as Lewy bodies. Lewy bodies are the pathological hallmark of the idiopathic disorder, and the distribution of the Lewy bodies throughout the Parkinsonian brain varies from one individual to another. The anatomical distribution of the Lewy bodies is often directly related to the expression and degree of the clinical symptoms of each individual. PD is also characterized by insufficient formation and activity of dopamine produced in certain neurons within parts of the midbrain.

There is no cure for PD, but medications and surgery can provide relief from the symptoms. Levodopa (L-DOPA, L-3,4-dihydroxyphenylalanine) has been the most widely used treatment for over 30 years. L-DOPA is converted into dopamine in the dopaminergic neurons by dopa decarboxylase. Since motor symptoms are produced by a lack of dopamine in the substantia nigra, the administration of L-DOPA temporarily diminishes the motor symptoms. Levodopa is usually combined with a dopa decarboxylase inhibitor or COMT inhibitor. The other main families of drugs useful for treating motor symptoms are dopamine agonists and monoamine oxidase B (MAO-B) inhibitors such as selegiline and rasagiline. MAO-B breaks down dopamine secreted by the dopaminergic neurons, and MAO-B inhibitors increase the level of dopamine in the basal ganglia by blocking its metabolism. The reduction in MAO-B activity results in increased L-DOPA in the striatum. See, The National Collaborating Centre for Chronic Conditions, ed. (2006), “Symptomatic pharmacological therapy in Parkinson's disease”, Parkinson's Disease. London: Royal College of Physicians. pp. 59-100.

PD is diagnosed from a patient's medical history and a neurological examination. There is no lab test that will clearly identify the disease, but brain scans are sometimes used to rule out disorders that could give rise to similar symptoms. A diagnosis of PD may be confirmed by administering levodopa and monitoring for relief of motor impairment. The finding of Lewy bodies in the midbrain on autopsy is usually considered proof that the person had Parkinson's disease. The progress of the illness over time may reveal that it is not Parkinson's disease, and some authorities recommend that the diagnosis be periodically reviewed. Jankovic, 2008, J. Neurol. Neurosurg. Psychiatr. 79 (4): 368-76. Although the diagnosis of PD is straightforward when patients have a classical presentation, differentiating PD from other forms of parkinsonism can be challenging early in the course of the disease, when signs and symptoms overlap with other syndromes. Tolosa et al., 2006, Lancet Neurol 5:75-86.

A number of rating scales are used for the evaluation of motor impairment and disability in patients with PD, but most of these scales have not been fully evaluated for validity and reliability. Ramaker et al., 2002, Mov Disord 17:867-76. The Hoehn and Yahr scale is commonly used to compare groups of patients and to provide gross assessment of disease progression, ranging from stage 0 (no signs of disease) to stage 5 (wheelchair bound or bedridden unless assisted). However, the reliability and validity of diagnostic criteria for PD have not been clearly established. de Rijk et al., 1997, Neurology 48:1277-81. Misdiagnosis of PD can arise for a number of reasons. For example, in a study of patients taking antiparkinsonian medication (n=402), the most common causes of misdiagnoses were essential tremor, Alzheimer's disease, and vascular parkinsonism. Tolosa, 2006, cited above. More than 25% of patients in the study did not respond to antiparkinsonian medication. In addition, many of the prominent features of PD (e.g., rigidity, gait disturbance, bradykinesia) may also occur as a result of normal aging or from comorbid and multifactorial medical conditions (e.g., diabetes, Parkinson's disease). Arvanitakis, et al., 2004, Neurology 63:996-1001.

Accordingly, there is an unmet need for improved diagnosis of PD. Molecular-based markers may address this need.

### SUMMARY OF THE INVENTION

Where applicable or not specifically disclaimed, any one of the embodiments described herein are contemplated to be able to combine with any other one or more embodiments, even though the embodiments are described under different aspects of the invention.

The platform technology described herein is useful for identifying markers of Parkinson's disease and markers useful in identifying stages of Parkinson's disease in a subject, e.g., a male subject or a female subject. This platform technology integrates molecular interactions within and across a hierarchy of models starting from a primary human cell based model to human clinical samples. This approach has led to the identification of biomarkers of Parkinson's disease. The instant application provides several novel biomarkers associated with Parkinson's disease, and which are useful in methods for diagnosing Parkinson's disease, determining the stage of Parkinson's disease, or monitoring the progression of Parkinson's disease.

The invention described herein is based, at least in part, on a novel, collaborative utilization of network biology, genomic, proteomic, metabolomic, transcriptomic, and bioinformatics tools and methodologies, which, when combined, may be used to study any biological system of interest, such as obtaining insight into the molecular mechanisms associated with or causal for Parkinson's disease.

In certain embodiments, the invention provides a method for diagnosing the presence of Parkinson's disease in a subject, or determining the stage of Parkinson's disease in a subject, comprising: (a) detecting the level of at least one of the markers in Table 2 or Table 5 in a biological sample of the subject, and (b) comparing the level of the marker in the biological sample with a predetermined threshold value, wherein an increased or decreased level of the marker as compared to the predetermined threshold value indicates the presence of Parkinson's disease in the subject.

In certain embodiments, the method further comprises detecting the level of one or more additional markers of Parkinson's disease.

In certain embodiments the biological sample is selected from the group consisting of blood, serum, urine, cerebrospinal fluid, organ tissue, feces, skin, hair, and cheek tissue.

In certain embodiments, the level of the marker is determined by immunoassay or ELISA.

In certain embodiments, the level of the marker is determined by mass spectrometry.

In certain embodiments, step (a) comprises (i) contacting the biological sample with a reagent that selectively binds to the marker to form a marker complex, and (ii) detecting the marker complex.

In certain embodiments, the stage of Parkinson's disease is based on the Hoehn-Yahr scale 0, scale 1, scale 1.5, scale 2, scale 2.5, scale 3, scale 4, or scale 5.

In certain embodiments, the method further comprises administering a treatment for Parkinson's disease where the diagnosis indicates the presence of Parkinson's disease in the subject.

In certain embodiments, the method further comprises selecting a subject suspected of having or being at risk of having Parkinson's disease.

In certain embodiments, the method further comprises obtaining a biological sample from a subject suspected of having or being at risk of having Parkinson's disease.

In certain embodiments, the method father comprises comparing the level of the at least one marker in the biological sample with the level of the at least one marker in a control sample selected from the group consisting of: a sample obtained from the same subject at an earlier time point than the biological sample, and a sample from a subject with Parkinson's disease.

In certain embodiments, the level of the at least one of the markers in Table 2 or Table 5 is increased as compared to the control.

In other embodiments, the level of the at least one of the markers in Table 2 or Table 5 is decreased as compared to the control.

The invention also provides a method for monitoring Parkinson's disease in a subject, the method comprising:
(1) determining a level of at least one of the markers in Table 2 or Table 5 in a first biological sample obtained at a first time from a subject having Parkinson's disease;
(2) determining the level of the at least one marker in a second biological sample obtained from the subject at a second time, wherein the second time is later than the first time; and
(3) comparing the level of the at least one marker in the second sample with the level of the at least one marker in the first sample, wherein a change in the level of the at least one marker is indicative of a change in Parkinson's disease status or stage in the subject.

In certain embodiments, the determining steps (1) and (2) further comprise determining the level of one or more additional markers in Table 2 or Table 5.

In certain embodiments, the subject is actively treated for Parkinson's disease prior to obtaining the second sample.

In certain embodiments, the subject is not actively treated for Parkinson's disease prior to obtaining the second sample.

In certain embodiments, a change in the level of the at least one marker and/or the one or more additional markers in the second biological sample as compared to the first biological sample is indicative of progression of Parkinson's disease in the subject.

In certain embodiments, a changed or equivalent level of the markers in Table 2 or Table 5 and/or the one or more additional markers in the second biological sample as compared to the first biological sample is indicative of non-progression of the Parkinson's disease in the subject.

In certain embodiments, the method further comprises comparing the level of the at least one Parkinson's disease markers in the first biological sample or the second biological sample with the level of the at least one marker in a control sample selected from the group consisting of a normal control sample and a sample from a subject with Parkinson's disease.

In certain embodiments, the method further comprises obtaining a first sample and a second sample from the subject.

In certain embodiments, the method further comprises selecting and/or administering a different treatment regimen for the subject based on progression of the Parkinson's disease in the subject.

In certain embodiments, the method further comprises administering a therapeutic for Parkinson's disease to the subject based on progression of the Parkinson's disease in the subject.

In certain embodiments, the method further comprises withholding an active treatment of the Parkinson's disease in the subject based on non-progression of the Parkinson's disease in the subject.

The invention also provides a method of treating Parkinson's disease in a subject, comprising: (a) obtaining a biological sample from a subject suspected of having Parkinson's disease, (b) submitting the biological sample to obtain diagnostic information as to the level of at least one of the markers in Table 2 or Table 5, (c) administering a therapeutically effective amount of a Parkinson's disease therapy if the level of the at least one marker is above or below a threshold level.

The invention also provides a method of treating Parkinson's disease in a subject, comprising: (a) obtaining diagnostic information as to the level of at least one of the markers in Table 2 or Table 5 in a biological sample, and (b) administering a therapeutically effective amount of a Parkinson's disease therapy if the level of the at least one marker is above or below a threshold level.

The invention also provides a method of treating Parkinson's disease in a subject, comprising: (a) obtaining a biological sample from a subject suspected of having Parkinson's disease for use in identifying diagnostic information as to the level of at least one of the markers in Table 2 or Table 5, (b) measuring the level of the at least one marker in the biological sample, (c) recommending to a healthcare provider to administer a Parkinson's disease therapy if the level of the at least one marker is above or below a threshold level.

In certain embodiments, the method further comprises obtaining diagnostic information as to the level of one or more additional markers of Parkinson's disease.

In certain embodiments, the method further comprises measuring the level of the one or more additional markers of Parkinson's disease.

In certain embodiments, the method further comprises administering a therapeutically effective amount of a Parkinson's disease therapy if the level of the at least one marker and at least one of the additional markers of Parkinson's disease are above or below a threshold level.

In certain embodiments, the method further comprises recommending to a healthcare provider to administer a Parkinson's disease therapy if the level of the at least one marker and at least one of the additional markers of Parkinson's disease are above or below a threshold level.

In certain embodiments, the biological sample is selected from the group consisting of blood, serum, urine, cerebrospinal fluid, organ tissue, feces, skin, hair, and cheek tissue.

In certain embodiments, the level of the at least one marker is determined by immunoassay or ELISA.

In certain embodiments, the level of the at least one marker is determined by mass spectrometry.

In certain embodiments, the level of the at least one marker is determined by (i) contacting the biological sample with a reagent that selectively binds to the at least one marker to form a marker complex, and (ii) detecting the marker complex.

The invention also provides a kit for detecting at least one of the markers in Table 2 or Table 5 in a biological sample, comprising at least one reagent for measuring the level of the at least one marker in the biological sample, and a set of instructions for measuring the level of the at least one marker.

The invention also provides a panel for use in a method of detecting at least two markers for Parkinson's disease, the panel comprising at least two detection reagents, wherein each detection reagent is specific for the detection of at least one Parkinson's disease marker of a set of markers, wherein the set of markers comprises at least two of the markers in Table 2 or Table 5.

The invention also provides a panel for use in a method of treating Parkinson's disease, the panel comprising at least two detection reagents, wherein each detection reagent is specific for the detection of at least one Parkinson's disease marker of a set of markers, wherein the set of markers comprises at least two of the markers in Table 2 or Table 5.

The invention also provides a panel for use in a method of monitoring the treatment of Parkinson's disease, the panel comprising at least two detection reagents, wherein each detection reagent is specific for the detection of at least one Parkinson's disease marker of a set of markers, wherein the set of markers comprises at least two of the markers in Table 2 or Table 5.

The invention also provides a kit comprising a panel of the invention and a set of instructions for obtaining diagnostic information as to the level of the at least two markers of Parkinson's disease.

The invention also provides use of a panel comprising a plurality of detection reagents specific for detecting markers of Parkinson's disease in a method for diagnosing and/or treating Parkinson's disease, wherein each detection reagent is specific for the detection of a marker in Table 2 or Table 5.

Where applicable or not specifically disclaimed, any one of the embodiments described herein are contemplated to be able to combine with any other one or more embodiments, even though the embodiments are described under different aspects of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts an overview of the bAIcis™ analysis to identify markers associated with Parkinson's disease.

FIG. 2 depicts bAIcis™ networks for all subjects, female subjects only, and male subjects only, which include the top 20 Parkinson's disease biomarkers identified by network analysis.

FIG. 3 depicts VIN subnetworks for all data.

FIG. 4 depicts VIN subnetworks for female subjects and male subjects.

FIG. 5 depicts biomarkers identified based on the network analysis as described in Example 1, including biomarkers deoxyinosine, phosphoserine, 1-methyladenosine, methylguanine, TRIM14, SGK223, PROS1, C4BPA, C4BPB, HP (haptoglobin), D-erythrose-4-phosphate, oxaloacetate/methysuccinate/ethylmalonic acid/glutaric acid (referred to as oxaloacetate), N-acetylputrescine, and kynurenine. Markers identified by circles are proteins.

FIG. 6 includes box plots depicting biomarker oxaloacetate/methysuccinate/ethylmalonic acid/glutaric acid (referred to as oxaloacetate) for PD vs. control for all subjects, PD vs. control for male subjects only, and PD vs. control for female subjects only. For each analysis, oxaloacetate/methysuccinate/ethylmalonic acid/glutaric acid is increased in PD vs. control samples. PD staging based on Hoehn-Yahr scale stages 1.0, 1.5, 2.0, 2.5, 3, and 4 for all subjects is also depicted. The controls for each analysis are on the left.

FIG. 7 includes box plots depicting biomarker 2-ketohexanoic acid for PD vs. control for all subjects, PD vs. control for male subjects only, and PD vs. control for female subjects only. For each analysis, 2-ketohexanoic acid is decreased in PD vs. control samples. PD staging based on Hoehn-Yahr scale stages 1.0, 1.5, 2.0, 2.5, 3, and 4 for all subjects is also depicted. The controls for each analysis are on the left.

FIG. 8 includes box plots depicting biomarker N-acetylputrescine for PD vs. control for all subjects, PD vs. control for male subjects only, and PD vs. control for female subjects only. For all analysis, N-acetylputrescine is increased in PD vs. control samples. PD staging based on Hoehn-Yahr scale stages 1.0, 1.5, 2.0, 2.5, 3, and 4 for all subjects is also depicted. The controls for each analysis are on the left.

FIG. 9 depicts staging (based on the Hoehn-Yahr scale) and PD vs. control for the “all subjects network” for various combinations of the biomarkers N-acetylputrescine, C4BPA, C4BPB, SGK223, HP, and PROS1.

FIG. 10A-D includes box plots for the following biomarkers: SL-9-HODE, AC-10-2, AC-10-3, and PE-36-6, indicating levels for each marker in PD vs. a control. Panel A shows PD vs. Control in all subjects. Panel B shows PD vs. Control in male subjects. Panel C shows PD vs. Control in female subjects. For panels A-C, the control shown is on the left (light gray), and the marker is shown on the right (dark gray). Panel D shows all subjects based on PD stages. For panel D, the control is shown on the far left, followed by stages 1.0, 1.5, 2, 2.5, 3, and 4, based on the Hoehn-Yahr scale.

FIG. 11A-D includes box plots for the following biomarkers: F5GZZ9 (CD163), P13591 (NCAM), Q14624.3 (ITIH4), BM000397 (N-Acetylputerscine), and oxaloacetate/methysuccinate/ethylmalonic acid/glutaric acid (referred to as BM000437 oxaloacetate), indicating levels for each marker in PD vs. a control. Panel A shows PD vs. Control in all subjects. Panel B shows PD vs. Control in male subjects. Panel C shows PD vs. Control in female subjects. For panels A-C, the control shown is on the left (light gray), and the marker is shown on the right (dark gray). Panel D shows all subjects based on PD stages. For panel D, the control is shown on the far left, followed by stages 1.0, 1.5, 2, 2.5, 3, and 4, based on the Hoehn-Yahr scale.

FIG. 12 depicts AUCs for PD vs. Control models for selected biomarkers.

FIG. 13 depicts ROC curves for PD vs. Control for biomarkers included in Table 5. “All markers” model includes each of the 9 biomarkers included in Table 5.

FIG. 14 shows AUC for PD subjects divided based on groups for biomarkers SL-9-HODE, BM000397 (N-acetylputerscine), oxaloacetate/methysuccinate/ethylmalonic acid/glutaric acid (referred to as BM000437 (oxaloacetate)), and PE-36-6.

FIG. 15 is a mass chromatogram illustrating HILIC-LC-MS/MS analysis of an oxaloacetic acid, methylsuccinic acid, ethylmalonic acid and glutaric acid mixture. No separation is observed.

FIG. 16 depicts the results of a metabolomic stability assessment of biomarkers methysuccinate and N-acetylputerscine during the course of a day (5 time points during the day) in healthy subjects. It was determined that in an assessment of healthy controls, methylsuccinate and N-acetylputrescine are metabolite biomarkers that do not change over the course of a day.

FIG. 17 depicts the results of a metabolomic stability assessment of biomarkers methysuccinate and N-acetylputerscine across five consecutive days in healthy subjects. It was determined that in an assessment of healthy controls, methylsuccinate and N-acetylputrescine are metabolite biomarkers that do not change over the course of 5 consecutive days.

FIGS. 18A-B depict the results of analyses of concomitant medications on the profile of PD biomarkers P13591 (NCAM), SL-9-HODE, methysuccinate, N-acetylputerscine, Q14624.3 (ITIH4), F5GZZ9 (CD163), AC-10:2, AC-10:3, and PE-36:6. (A) Depicts the impact of dopamine replacement medications containing levodopa and COMT inhibitors (e.g., Entac) on the biomarkers. (B) Depicts the impact of dopamine agonist medication on the biomarkers. Normal=non-diseased controls, individuals without PD; Never=PD patients that have never been exposed to that drug; Ever=PD patients that at one time were exposed to the drug; Current=PD patients that are currently taking the drug. The number of subjects in each category are listed in the tables for both (A) and (B).

FIGS. 19A-B depict the results of analyses of concomitant medications on the profile of PD biomarkers P13591 (NCAM), SL-9-HODE, methysuccinate, N-acetylputerscine, Q14624.3 (ITIH4), F5GZZ9 (CD163), AC-10:2, AC-10:3, and PE-36:6. (A) Depicts the impact of both dopamine replacement and dopamine agonist medication on the biomarkers. (B) Depicts the impact of MAOB inhibitors on the biomarkers. Normal=non-diseased controls, individuals without PD; Never=PD patients that have never been exposed to that drug; Ever=PD patients that at one time were exposed to the drug; Current=PD patients that are currently taking the drug. The number of subjects in each category are listed in the tables for both (A) and (B).

FIGS. 20A-B depict the results of analyses of concomitant medications on the profile of PD biomarkers P13591 (NCAM), SL-9-HODE, methysuccinate, N-acetylputerscine, Q14624.3 (ITIH4), F5GZZ9 (CD163), AC-10:2, AC-10:3, and PE-36:6. (A) Depicts an analysis of the impact on the biomarkers in patients that are early in the disease process and have only taken MAOB inhibitors and have never taken dopamine replacement or dopamine agonist medication. (B) Depicts the impact of Amantadine, an antiparkinsonian drug, on the biomarkers. Normal=non-diseased controls, individuals without PD; Never=PD patients that have never been exposed to that drug; Ever=PD patients that at one time were exposed to the drug; Current=PD patients that are currently taking the drug. The number of subjects in each category are listed in the tables for both (A) and (B).

FIG. 21 is a multi-omics biomarker panel analysis including biomarkers methysuccinate, N-acetylputerscine, and SL-9-HODE. The AUC for all patients (0.75), female patients (0.72), and male patients (0.77) are set forth in the table.

FIG. 22 is a multi-omics biomarker panel combination with clinical features, which includes the biomarker methysuccinate in combination with the clinical features set forth in the table. The AUC for all patients is 0.95. Clinical features listed in the table are: BSitTotal—combined score from the smell test; HADSDTotal—Total Depression Score; MedicalHistory NeuACT2—Neurologic Condition 2 Active; Age—age; RBDRBDNO2—not acting out dreams while asleep; MedicalHistoryMUSCAT2—Musculoskeletal Condition 2 Active; MedicalHistoryPULMYES—Pulmonary condition; MedicalHistoryHEMAL1RES—Hematolymphatic Condition 1 Resolved; and MedicalHistoryOTHERACT3—OTHER condition 3 Active.

FIG. 23 is a multi-omics biomarker panel combination with clinical features, which includes the biomarkers methylsuccinate and N-acetylputerscine in combination with the clinical features set forth in the table. The AUC for all patients is 0.7. Clinical features listed in the table are: BSitTotal—combined score from the smell test; HADSDTotal—Total Depression Score; RBDRBDNO2—not acting out dreams while asleep; MedicalHistoryHEMAL1RES—Hematolymphatic Condition 1 Resolved; and MedicalHistoryENTRES1—ENT Condition 1 Resolved.

FIGS. 24A-24C are ROC curves representing the diagnostic value of molecular markers in differentiating individuals with and without Parkinson's Disease (FIG. 24A) and staging Parkinson's Disease (FIG. 24B), as well as ROC curves representing diagnostic value of molecular marker and clinical variable combinations for differentiating individuals with and without Parkinson's disease.

FIGS. 25A-25B are spectra obtained using an earlier, metabolomics-based method (FIG. 25A) and an LC-MS/MS method as described herein (FIG. 25B) for detecting the biomarkers MSA, EMA and GA in plasma.

FIGS. 26A-26C are sensitivity diagrams comparing the level and range of detection achieved for the biomarkers MSA (FIG. 26A), EMA (FIG. 26B) and GA (FIG. 26C) using known methods and using an LC-MS/MS as described herein.

FIGS. 27A-27B are spectra obtained using an earlier, metabolomics-based method (FIG. 27A) and an LC-MS/MS method as described herein (FIG. 27B) for detecting NAP in plasma.

FIGS. 28A-28E are graphical summaries of validation results obtained for precision analyses (FIG. 28A), inter-assay analyses (FIG. 28B), accuracy analyses (FIG. 28C), parallelism analyses (FIG. 28D), and short-term stability analyses (FIG. 28E) performed using the NCAM detection and quantitation methods of the invention.

FIGS. 29A-29F are graphical summaries of validation results obtained for precision analyses (FIGS. 29A-29B), inter-assay analyses (FIG. 29C), accuracy analyses (FIG. 29D), parallelism analyses (FIG. 29E), and short-term stability analyses (FIG. 29F) performed using the ITIH4 detection and quantitation methods of the invention.

FIGS. 30A-30C are scatterplots for the biomarkers EMA (FIG. 30A), GA (FIG. 30B), and MSA (FIG. 30C) in log 2 versus normalized oxaloacetate.

FIGS. 31A-31F are ROC curves for the biomarkers EMA (FIG. 31A), GA (FIG. 31B), MSA (FIG. 31C), NAP (FIG. 31D), NCAM (FIG. 31E), and ITIH4 (FIG. 31F). For each ROC curve, AUC value is presented along with the number of samples in each group, where N1 and NO is the number of samples in PD and Healthy groups, respectively.

FIGS. 32A-32D are an ROC curve (FIG. 32A), diagnostic tables (FIG. 32B and FIG. 32C) and a Beeswarm plot of NAP vs. PD (FIG. 32D) summarizing diagnostic assessment results for the biomarker NAP.

FIGS. 33A-33F are ROC curves for age (FIG. 33A), smell test (BSitTotal, FIG. 33B), and anxiety test (HADsDTotal, FIG. 33C); bar graphs of the proportion distribution of RBDNO results in the presence or absence of Parkinson's Disease (FIG. 33D); and Beeswarm plots for the smell test (BSitTotal vs. PD, FIG. 33E) and anxiety test (HADsDTotal vs. PD, FIG. 33F) representing diagnostic assessment results for select clinical variables.

FIGS. 34A-34C are an ROC curve (FIG. 34A) and diagnostic tables (FIG. 34B and FIG. 34C) summarizing diagnostic assessment results for the combination of biomarkers NAP and EMA.

FIGS. 35A-35C are an ROC curve (FIG. 35A) and diagnostic tables (FIG. 35B and FIG. 35C) summarizing diagnostic assessment results for the NAP+BsitTotal+HADsDTotal+age combination.

FIGS. 36A-36C are an ROC curve (FIG. 36A) and diagnostic tables (FIG. 36B and FIG. 36C) summarizing diagnostic assessment results for the NAP+BsitTotal+HADsDTotal+RBDNO combination.

FIGS. 37A-37C depict plasma sample analysis for N-Acetylputrescine (NAP) and receiver operation characteristic (ROC) curve analysis. FIG. 2A shows plasma levels of NAP between non-disease and PD cohort (left) and ROC curve analysis for NAP alone (right). FIG. 2B shows ROC curve analysis for NAP plus three clinical variables. FIG. 2C shows summary table for clinical performance of marker panel alone and combination, including area under curve (AUC), sensitivity, specificity, positive predictive value (PPV), negative predictive valve (NPV), and odds ratio (OR). 95% confident interval (CI) using Bootstrapping approach in ROC curve. Statistics was calculated by t-test, statistically significant: ****p<0.0001

### DETAILED DESCRIPTION OF THE INVENTION

### A. Overview

As presently described herein, the present invention is based, at least in part, on the discovery that the levels of the markers listed in Table 2 or Table 5, are modulated in subjects having Parkinson's disease, and across various stages of the disease, and thus serve as useful markers of Parkinson's disease and markers of stages of Parkinson's disease. The invention is also based on the discovery that the markers listed in Table 2 or Table 5, alone in combination with one or more of an anxiety test, a sleep test, a smell test, or any combination thereof, can be useful for as diagnostic or prognostic markers for Parkinson's disease. In one embodiment, one or more of the markers listed in Table 2 or Table 5, and optionally, performance on an anxiety test, a sleep test, a smell test, or any combination thereof, can serve as useful diagnostic marker(s) to predict and/or detect the presence of Parkinson's disease in a subject, or the stage of progression of the disease. In another embodiment, one or more of the markers listed in Table 2 or Table 5, and optionally, performance on an anxiety test, a sleep test, a smell test, or any combination thereof can serve as useful prognostic marker(s), serving to inform on the likely progression of Parkinson's disease in a subject with or without treatment. In still another embodiment, one or more of the markers listed in Table 2 or Table 5, and optionally, performance on an anxiety test, a sleep test, a smell test, or any combination thereof can serve as useful predictive marker(s) for helping to assess the likely response of Parkinson's disease to a particular treatment.

Accordingly, the invention provides methods that use markers, e.g., the markers listed in Table 2 or Table 5, and optionally, performance on an anxiety test, a sleep test, a smell test, or any combination thereof, in the diagnosis of Parkinson's disease (e.g., prediction of the presence of Parkinson's disease in a subject), in the diagnosis of the stage of Parkinson's disease (e.g., diagnosis of the stage of Parkinson's disease in a subject), in the prognosis of Parkinson's disease (e.g., prediction of the course or outcome of Parkinson's disease with or without treatment), and in the assessment of therapies intended to treat Parkinson's disease (i.e., the markers listed in Table 2 or Table 5, and optionally, an anxiety test, a sleep test, a smell test, or any combination thereof as a theragnostic or predictive marker). The invention further provides compositions of matter, including panels comprising binding or detection reagents specific for the markers listed in Table 2 or Table 5 and optionally other markers for use in the methods of the invention, as well as kits for practicing the methods of the invention.

As presently described herein, the present invention is also based, at least in part, on the discovery that the levels of N-acetyl putrescine (NAP), ethyl malonic acid (EMA), or both NAP and EMA together, are modulated in subjects having Parkinson's disease, and across various stages of the disease, and thus serve as useful markers of Parkinson's disease and markers of stages of Parkinson's disease. The invention is also based on the discovery that NAP, EMA, or NAP and EMA, in combination with one or more of an anxiety test, a sleep test, a smell test, or any combination thereof, can be useful for as diagnostic or prognostic markers for Parkinson's disease. In one embodiment, one or more of the biomarkers NAP, EMA, or NAP and EMA, and optionally, performance on an anxiety test, a sleep test, a smell test, or any combination thereof can serve as useful diagnostic marker(s) to predict and/or detect the presence of Parkinson's disease in a subject, or the stage of progression of the disease. In another embodiment, the biomarker NAP, EMA, or NAP and EMA, and optionally, performance on an anxiety test, a sleep test, a smell test, or any combination thereof can serve as useful prognostic marker(s), serving to inform on the likely progression of Parkinson's disease in a subject with or without treatment. In still another embodiment, the biomarker NAP, EMA, or NAP and EMA, and optionally, performance on an anxiety test, a sleep test, a smell test, or any combination thereof can serve as useful predictive marker(s) for helping to assess the likely response of Parkinson's disease to a particular treatment.

Accordingly, the invention provides methods that use markers, e.g., NAP, EMA, or NAP and EMA, and optionally, performance on an anxiety test, a sleep test, a smell test, or any combination thereof, in the diagnosis of Parkinson's disease (e.g., prediction of the presence of Parkinson's disease in a subject), in the diagnosis of the stage of Parkinson's disease (e.g., diagnosis of the stage of Parkinson's disease in a subject), in the prognosis of Parkinson's disease (e.g., prediction of the course or outcome of Parkinson's disease with or without treatment), and in the assessment of therapies intended to treat Parkinson's disease (i.e., NAP, EMA, NAP and EMA, and optionally, an anxiety test, a sleep test, a smell test, or any combination thereof as a theragnostic or predictive marker). The invention further provides compositions of matter, including panels comprising binding or detection reagents specific for NAP, EMA, NAP and EMA and optionally other markers for use in the methods of the invention, as well as kits for practicing the methods of the invention.

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

### B. Definitions

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

As used herein, the singular forms “a”, “and”, and “the” include plural references unless the context clearly dictates otherwise. All technical and scientific terms used herein have the same meaning.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “amplification” refers to any known in vitro procedure for obtaining multiple copies (“amplicons”) of a target nucleic acid sequence or its complement or fragments thereof. In vitro amplification refers to production of an amplified nucleic acid that may contain less than the complete target region sequence or its complement. Known in vitro amplification methods include, e.g., transcription-mediated amplification, replicase-mediated amplification, polymerase chain reaction (PCR) amplification, ligase chain reaction (LCR) amplification and strand-displacement amplification (SDA including multiple strand-displacement amplification method (MSDA)). Replicase-mediated amplification uses self-replicating RNA molecules, and a replicase such as Q-β-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Two other known strand-displacement amplification methods do not require endonuclease nicking (Dattagupta et al., U.S. Pat. Nos. 6,087,133 and 6,124,120 (MSDA)). Those skilled in the art will understand that the oligonucleotide primer sequences of the present invention may be readily used in any in vitro amplification method based on primer extension by a polymerase. (see generally Kwoh et al., 1990, Am. Biotechnol. Lab. 8:14-25 and (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 2000, Molecular Cloning—A Laboratory Manual, Third Edition, CSH Laboratories). As commonly known in the art, the oligos are designed to bind to a complementary sequence under selected conditions.

As used herein, the term “antigen” refers to a molecule, e.g., a peptide, polypeptide, protein, fragment, or other biological moiety, which elicits an antibody response in a subject, or is recognized and bound by an antibody.

As used herein, the term “area under the curve” or “AUC” refers to the area under the curve in a plot of sensitivity versus specificity. In one embodiment, the AUC for a biomarker, or combination of biomarkers, of the invention is 0.5. In another embodiment, the AUC for a biomarker, or combination of biomarkers, of the invention is 0.6. In another embodiment, the AUC for a biomarker, or combination of biomarkers, of the invention is 0.7. In another embodiment, the AUC for a biomarker, or combination of biomarkers, of the invention is 0.8. In another embodiment, the AUC for a biomarker, or combination of biomarkers, of the invention is 0.9. In another embodiment, the AUC for a biomarker, or combination of biomarkers, of the invention is 1.0. In specific embodiments, the AUC for a biomarker, or combination of biomarkers, of the invention is 0.5, 0.51, 0.52, 0.53, 0.54, 0.55, 0.56, 0.57, 0.58, 0.59, 0.6, 0.61, 0.62, 0.63, 0.64, 3.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99 or 1.0. In one embodiment, the AUC for a biomarker, or combination of biomarkers, of the invention is at least 0.5. In another embodiment, the AUC for a biomarker, or combination of biomarkers, of the invention is at least 0.6. In another embodiment, the AUC for a biomarker, or combination of biomarkers, of the invention is at least 0.7. In another embodiment, the AUC for a biomarker, or combination of biomarkers, of the invention is at least 0.8. In another embodiment, the AUC for a biomarker, or combination of biomarkers, of the invention is at least 0.9. In another embodiment, the AUC for a biomarker, or combination of biomarkers, of the invention is at least 1.0. In specific embodiments, the AUC for a biomarker, or combination of biomarkers, of the invention is at least 0.5, 0.51, 0.52, 0.53, 0.54, 0.55, 0.56, 0.57, 0.58, 0.59, 0.6, 0.61, 0.62, 0.63, 0.64, 3.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99 or 1.0

As used herein, the term “biomarker” or “marker” is understood to mean a measurable characteristic that reflects in a quantitative or qualitative manner the physiological state of an organism. The physiological state of an organism is inclusive of any disease or non-disease state, e.g., a subject having Parkinson's disease or a subject who is otherwise healthy. Said another way, markers are characteristics that can be objectively measured and evaluated as indicators of normal processes, pathogenic processes, or pharmacologic responses to a therapeutic intervention. Markers can be clinical parameters (e.g., age, performance status such as that on an anxiety test, a sleep test, or a smell test), laboratory measures (e.g., molecular markers), imaging-based measures, or genetic or other molecular determinants, such as phosphorylation or acetylation state of a protein marker, methylation state of nucleic acid, or any other detectable molecular modification to a biological molecule. Examples of markers include, for example, polypeptides, peptides, polypeptide fragments, proteins, antibodies, hormones, polynucleotides, RNA or RNA fragments, microRNA (miRNAs), lipids (e.g., structural lipids or signaling lipids), polysaccharides, and other bodily metabolites. In one embodiment, a biomarker of the invention is NAP, EMA, and/or one or more of the biomarkers included in Table 2 or Table 5. In another embodiment, a biomarker of the invention is one that is metabolically stable over time (e.g., over the course of 1, 2, 3, 4, 5, 6, 7, or more days), and is metabolically stable regardless of the diet of the subject. In still another embodiment, a biomarker of the invention is one that has a consistent biomarker profile regardless of whether or not the patient had been previously or is currently taking medications for PD or a related disease or disorder.

Preferably, a marker of the present invention is modulated (e.g., increased or decreased level) in a biological sample from a subject or a group of subjects having a first phenotype (e.g., having a disease) as compared to a biological sample from a subject or group of subjects having a second phenotype (e.g., not having the disease, e.g., a control). A marker may be differentially present at any level, but is generally present at a level that is increased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A marker is preferably differentially present at a level that is statistically significant (e.g., a p-value less than 0.05 and/or a q-value of less than 0.10 as determined using either Welch's T-test or Wilcoxon's rank-sum Test).

As used herein, the term “clinical parameter” or “clinical feature”, used interchangeably herein, includes any clinical measure of a disease state of a patient. Clinical parameters for PD can include, but are not limited to, assessment of gait, bradykinesia/hypokinesia, tremor, sleep, balance and cognition. Clinical parameters for PD can also include, but are not limited to, age, performance status, combined score from the smell test, total depression score, Neurologic Condition 2 Active, not acting out dreams while asleep, Musculoskeletal Condition 2 Active, pulmonary condition, Hematolymphatic Condition 1 Resolved, condition 3 Active, ENT Condition 1 Resolved, and recent medications. Other clinical parameters include Falls and Near Falls; Capability to Perform Activities of Daily Living; Interference with Activities of Daily Living; Capability to Process Tasks; Capability to Recall and Retrieve Information; Walkers; the Evaluation of Performance Doing Fine Motor Movements; Capability to Eat; Assessment of Sleep Quality; Identification of Circumstances and Triggers for Loose of Balance and Memory Assessment. Other clinical parameters, or symptoms, of PD, are discussed herein such as performance on the anxiety test as reflected in a HADsDTotal score, performance on the sleep test as reflected in the absence or presence of REM sleep behavior disorder, performance on the smell test as reflected in a BsitTotal score, or any combination thereof. One or more clinical features can be assessed in combination with one or more of the biomarkers such as NAP and EMA, as well as those set forth in Tables 2 and 5, for use in the methods of the invention.

As used herein, the term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control sample,” as used herein, refers to any clinically relevant comparative sample, including, for example, a sample from a healthy subject not afflicted with Parkinson's disease, or a sample from a subject from an earlier time point, e.g., prior to treatment, an earlier assessment time point, at an earlier stage of treatment. A control sample can be a purified sample, metabolite, lipid, protein, and/or nucleic acid provided with a kit. Such control samples can be diluted, for example, in a dilution series to allow for quantitative measurement of levels of analytes, e.g., markers, in test samples. A control sample may include a sample derived from one or more subjects. A control sample may also be a sample made at an earlier time point from the subject to be assessed. For example, the control sample could be a sample taken from the subject to be assessed before the onset of a disorder, e.g., Parkinson's disease, at an earlier stage of disease, or before the administration of treatment or of a portion of treatment. The control sample may also be a sample from an animal model, or from a tissue or cell line derived from the animal model of a disorder, e.g., Parkinson's disease. The level of activity or expression of one or more markers (e.g., 1, 2, 3, 4, 5, 6, 7, 8, 9, 10, 11, 12, 13, 14, 15, 16, 17, 18, 19, 20, 21, 22, or more markers) in a control sample consists of a group of measurements that may be determined, e.g., based on any appropriate statistical measurement, such as, for example, measures of central tendency including average, median, or modal values. Different from a control is preferably statistically significantly different from a control.

As used herein, “changed as compared to a control” sample or subject is understood as having a level of the analyte or diagnostic or therapeutic indicator (e.g., marker) to be detected at a level that is statistically different than a sample from a normal, untreated, or abnormal state control sample. Changed as compared to control can also include a difference in the rate of change of the level of one or more markers obtained in a series of at least two subject samples obtained over time. Determination of statistical significance is within the ability of those skilled in the art and can include any acceptable means for determining and/or measuring statistical significance, such as, for example, the number of standard deviations from the mean that constitute a positive or negative result, an increase in the detected level of a biomarker in a sample (e.g., Parkinson's Disease sample) versus a control or healthy sample, wherein the increase is above some threshold value, or a decrease in the detected level of a biomarker in a sample (e.g., Parkinson's Disease sample) versus a control or healthy sample, wherein the decrease is below some threshold value. The threshold value can be determine by any suitable means by measuring the biomarker levels in a plurality of tissues or samples known to have a disease, e.g., Parkinson's Disease, and comparing those levels to a normal sample and calculating a statistically significant threshold value.

The term “control level” refers to an accepted or pre-determined level of a marker in a subject sample. A control level can be a range of values. Marker levels can be compared to a single control value, to a range of control values, to the upper level of normal, or to the lower level of normal as appropriate for the assay. In one embodiment, the control is a standardized control, such as, for example, a control predetermined using an average of the levels of expression of one or more markers from a population of subjects having no Parkinson's disease.

In one embodiment, the control is a standardized control, such as, for example, a control predetermined using an average of the levels of expression of one or more markers from a population of subjects not having PD. A control can also be a sample from a subject at an earlier time point, e.g., a baseline level prior to suspected presence of disease, before the diagnosis of a disease, before the treatment with a specific agent (e.g., levodopa) or intervention (e.g., surgery). In certain embodiments, a change in the level of the marker in a subject can be more significant than the absolute level of a marker, e.g., as compared to control.

As used herein, “detecting”, “detection”, “determining”, and the like are understood to refer to an assay performed for identification of one or more specific markers in a sample, e.g., NAP, EMA, NAP and EMA, and/or one or more (e.g., 1, 2, 3, 4, 5, 6, 7, 8, 9, 10, 11, 12, 13, 14, 15, 16, 17, 18, 19, 20, 21, 22 or more) markers selected from the group consisting of the markers in Table 2 and Table 5. The amount of the marker detected in the sample can be none or below the level of detection of the assay or method.

As used herein, the term “DNA” or “RNA” molecule or sequence (as well as sometimes the term “oligonucleotide”) refers to a molecule comprised generally of the deoxyribonucleotides adenine (A), guanine (G), thymine (T) and/or cytosine (C). In “RNA”, T is replaced by uracil (U).

The terms “disorders”, “diseases”, and “abnormal state” are used inclusively and refer to any deviation from the normal structure or function of any part, organ, or system of the body (or any combination thereof). A specific disease is manifested by characteristic symptoms and signs, including biological, chemical, and physical changes, and is often associated with a variety of other factors including, but not limited to, demographic, environmental, employment, genetic, and medically historical factors. Certain characteristic signs, symptoms, and related factors can be quantitated through a variety of methods to yield important diagnostic information. As used herein the disorder, disease, or abnormal state is Parkinson's disease.

As used herein, a sample obtained at an “earlier time point” is a sample that was obtained at a sufficient time in the past such that clinically relevant information could be obtained in the sample from the earlier time point as compared to the later time point. In certain embodiments, an earlier time point is at least four weeks earlier. In certain embodiments, an earlier time point is at least six weeks earlier. In certain embodiments, an earlier time point is at least two months earlier. In certain embodiments, an earlier time point is at least three months earlier. In certain embodiments, an earlier time point is at least six months earlier. In certain embodiments, an earlier time point is at least nine months earlier. In certain embodiments, an earlier time point is at least one year earlier. Multiple subject samples (e.g., 3, 4, 5, 6, 7, or more) can be obtained at regular or irregular intervals over time and analyzed for trends in changes in marker levels. Appropriate intervals for testing for a particular subject can be determined by one of skill in the art based on ordinary considerations.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

As used herein, “greater predictive value” is understood as an assay that has significantly greater sensitivity and/or specificity, preferably greater sensitivity and specificity, than the test to which it is compared. The predictive value of a test can be determined using an ROC analysis. In an ROC analysis a test that provides perfect discrimination or accuracy between normal and disease states would have an area under the curve (AUC)=1, whereas a very poor test that provides no better discrimination than random chance would have AUC=0.5. As used herein, a test with a greater predictive value will have a statistically improved AUC as compared to another assay. The assays are performed in an appropriate subject population.

A “higher level of expression”, “higher level”, and the like of a marker refers to an expression level in a test sample that is greater than the standard error of the assay employed to assess expression, and is preferably at least 25% more, at least 50% more, at least 75% more, at least two, at least three, at least four, at least five, at least six, at least seven, at least eight, at least nine, or at least ten times the expression level of the marker in a control sample (e.g., sample from a healthy subject not having the marker associated disease, i.e., Parkinson's disease) and preferably, the average expression level of the marker or markers in several control samples.

As used herein, the term “hybridization,” as in “nucleic acid hybridization,” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 2000, supra and Ausubel et al., 1994, supra, or further in Higgins and Hames (Eds.) “Nucleic acid hybridization, a practical approach” IRL Press Oxford, Washington D.C., (1985)) and are commonly known in the art. In the case of a hybridization to a nitrocellulose filter (or other such support like nylon), as for example in the well-known Southern blotting procedure, a nitrocellulose filter can be incubated overnight at a temperature representative of the desired stringency condition (60-65° C. for high stringency, 50-60° C. for moderate stringency and 40-45° C. for low stringency conditions) with a labeled probe in a solution containing high salt (6×SSC or 5×SSPE), 5×Denhardt's solution, 0.5% SDS, and 100 μg/ml denatured carrier DNA (e.g., salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The salt and SDS concentration of the washing solutions may also be adjusted to accommodate for the desired stringency. The selected temperature and salt concentration is based on the melting temperature (Tm) of the DNA hybrid. Of course, RNA-DNA hybrids can also be formed and detected. In such cases, the conditions of hybridization and washing can be adapted according to well-known methods by the person of ordinary skill. Stringent conditions will be preferably used (Sambrook et al., 2000, supra). Other protocols or commercially available hybridization kits (e.g., ExpressHyb® from BD Biosciences Clonetech) using different annealing and washing solutions can also be used as well known in the art. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed).

As used herein, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands. Moreover, the present invention also relates to nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid. The present invention also relates to nucleic acid molecules which comprise one or more mutations or deletions, and to nucleic acid molecules which hybridize to one of the herein described nucleic acid molecules, which show (a) mutation(s) or (a) deletion(s).

The term “including” is used herein to mean, and is used interchangeably with, the phrase “including but not limited to.”

A subject at “increased risk for developing Parkinson's disease” may or may not develop PD. Identification of a subject at increased risk for developing PD should be monitored for additional signs or symptoms of PD. The methods provided herein for identifying a subject with increased risk for developing PD can be used in combination with assessment of other known risk factors or signs of PD.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, a “label” refers to a molecular moiety or compound that can be detected or can lead to a detectable signal. A label is joined, directly or indirectly, to a molecule, such as an antibody, a nucleic acid probe or the protein/antigen or nucleic acid to be detected (e.g., an amplified sequence). Direct labeling can occur through bonds or interactions that link the label to the nucleic acid (e.g., covalent bonds or non-covalent interactions), whereas indirect labeling can occur through the use of a “linker” or bridging moiety, such as oligonucleotide(s) or small molecule carbon chains, which is either directly or indirectly labeled. Bridging moieties may amplify a detectable signal. Labels can include any detectable moiety (e.g., a radionuclide, ligand such as biotin or avidin, enzyme or enzyme substrate, reactive group, chromophore such as a dye or colored particle, luminescent compound including a bioluminescent, phosphorescent or chemiluminescent compound, and fluorescent compound). Preferably, the label on a labeled probe is detectable in a homogeneous assay system, i.e., in a mixture, the bound label exhibits a detectable change compared to an unbound label.

The terms “level of expression of a gene”, “gene expression level”, “level of a marker”, and the like refer to the level of mRNA, as well as pre-mRNA nascent transcript(s), transcript processing intermediates, mature mRNA(s) and degradation products, or the level of protein, encoded by the gene in the cell. The “level” of one of more biomarkers means the absolute or relative amount or concentration of the biomarker in the sample.

A “lower level of expression” or “lower level” of a marker refers to an expression level in a test sample that is less than 90%, 85%, 80%, 75%, 70%, 65%, 60%, 55%, 50%, 45%, 40%, 35%, 30%, 25%, 20%, 15%, or 10% of the expression level of the marker in a control sample (e.g., sample from a healthy subjects not having the marker associated disease, i.e., Parkinson's disease) and preferably, the average expression level of the marker in several control samples.

The term “modulation” refers to upregulation (i.e., activation or stimulation), down-regulation (i.e., inhibition or suppression) of a response (e.g., level of expression of a marker), or the two in combination or apart. A “modulator” is a compound or molecule that modulates, and may be, e.g., an agonist, antagonist, activator, stimulator, suppressor, or inhibitor.

As used herein, “negative fold change” refers to “down-regulation” or “decrease (of expression)” of a gene that is listed herein.

As used herein, “nucleic acid molecule” or “polynucleotides”, refers to a polymer of nucleotides. Non-limiting examples thereof include DNA (e.g., genomic DNA, cDNA), RNA molecules (e.g., mRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the term “nucleic acid” and polynucleotides as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Intl Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs). An “isolated nucleic acid molecule”, as is generally understood and used herein, refers to a polymer of nucleotides, and includes, but should not limited to DNA and RNA. The “isolated” nucleic acid molecule is purified from its natural in vivo state, obtained by cloning or chemically synthesized.

As used herein, the term “obtaining” is understood herein as manufacturing, purchasing, or otherwise coming into possession of.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural rare or synthetic nucleotides. They can be designed to enhance a chosen criteria like stability for example. Chimeras of deoxyribonucleotides and ribonucleotides may also be within the scope of the present invention.

As used herein, “one or more” is understood as each value 1, 2, 3, 4, 5, 6, 7, 8, 9, 10, and any value greater than 10.

The term “or” is used inclusively herein to mean, and is used interchangeably with, the term “and/or,” unless context clearly indicates otherwise. For example, as used herein, filamin B or LY9 is understood to include filamin B alone, LY9 alone, and the combination of filamin B and LY9.

As used herein, “patient” or “subject” can mean either a human or non-human animal, preferably a mammal. By “subject” is meant any animal, including horses, dogs, cats, pigs, goats, rabbits, hamsters, monkeys, guinea pigs, rats, mice, lizards, snakes, sheep, cattle, fish, and birds. A human subject may be referred to as a patient. It should be noted that clinical observations described herein were made with human subjects and, in at least some embodiments, the subjects are human.

As used herein, “positive fold change” refers to “up-regulation” or “increase (of expression)” of a gene that is listed herein.

As used herein, “preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). Prevention does not require that the disease or condition never occurs in the subject. Prevention includes delaying the onset or severity of the disease or condition.

As used herein, a “predetermined threshold value” or “threshold value” of a biomarker refers to the level of the biomarker (e.g., the expression level or quantity (e.g., ng/ml) in a biological sample) in a corresponding control/normal sample or group of control/normal samples obtained from normal or healthy subjects, e.g., those subjects that do not have Parkinson's Disease. The predetermined threshold value may be determined prior to or concurrently with measurement of marker levels in a biological sample. The control sample may be from the same subject at a previous time or from different subjects.

As used herein, a “probe” is meant to include a nucleic acid oligomer or oligonucleotide that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence or it can also be synthesized. Numerous primers and probes which can be designed and used in the context of the present invention can be readily determined by a person of ordinary skill in the art to which the present invention pertains.

As used herein, the terminology “prognosis”, “staging” and “determination of aggressiveness” are defined herein as the prediction of the degree of severity of the Parkinson's Disease and of its evolution as well as the prospect of increasing severity of symptoms as anticipated from usual course of the disease.

As used herein, “prophylactic” or “therapeutic” treatment refers to administration to the subject of one or more agents or interventions to provide the desired clinical effect. If it is administered prior to clinical manifestation of the unwanted condition (e.g., disease or other unwanted state of the host animal) then the treatment is prophylactic, i.e., it protects the host against developing at least one sign or symptom of the unwanted condition, whereas if administered after manifestation of the unwanted condition, the treatment is therapeutic (i.e., it is intended to diminish, ameliorate, or maintain at least one sign or symptom of the existing unwanted condition or side effects therefrom).

Parkinson's disease (PD) is a progressive neurological disorder characterized by a large number of motor and non-motor features that can impact function to a variable degree. Because there is no definitive test for the diagnosis of PD, the disease must be diagnosed based on clinical criteria, including rest tremor, bradykinesia, rigidity and loss of postural reflexes. The presence and specific presentation of these features are used to differentiate PD from related parkinsonian disorders. Other clinical features include secondary motor symptoms (e.g., hypomimia, dysarthria, dysphagia, sialorrhoea, micrographia, shuffling gait, festination, freezing, dystonia, glabellar reflexes), and non-motor symptoms (e.g., autonomic dysfunction, cognitive/neurobehavioral abnormalities, sleep disorders and sensory abnormalities such as anosmia, paresthesias and pain). Absence of rest tremor, early occurrence of gait difficulty, postural instability, dementia, hallucinations, and the presence of dysautonomia, ophthalmoparesis, ataxia and other atypical features, coupled with poor or no response to levodopa, suggest diagnoses other than PD. Jankovic, 2008, J. Neurol. Neurosurg. Psychiatr. 79 (4): 368-76.

Parkinsonian disorders can be classified as four types: primary (idiopathic) parkinsonism, secondary (acquired, symptomatic) parkinsonism, heredodegenerative parkinsonism and multiple system degeneration (parkinsonism plus syndromes). Several features, such as tremor, early gait abnormality (e.g., freezing), postural instability, pyramidal tract findings and response to levodopa, can be used to differentiate PD from other parkinsonian disorders. Jankovic, cited above.

Present clinical practice typically requires the presence of at least one primary motor symptom for a diagnosis of PD (See U.S. Pat. No. 8,778,334). The primary motor symptoms are:
(i) Resting Tremor: About 70 percent of people with Parkinson's experience a slight tremor, which is often the first identifiable symptom. The tremor is typically in either the hand or foot on one side of the body, or less commonly in the jaw or face. The Parkinson's tremor usually appears when a person's muscles are relaxed, hence it is called “resting tremor.”
(ii) Bradykinesia (Slow movement): the patient displays markedly slow movement. In addition to slow movement, a person with bradykinesia will typically also have incomplete movement, difficulty initiating movements and difficulty in suddenly stopping ongoing movements. People who have bradykinesia may walk with short, shuffling steps (festination). Bradykinesia and rigidity can occur in the facial muscles, reducing a person's range of facial expressions and resulting in a “mask-like” appearance.
(iii) Rigidity: also called increased muscle tone, means stiffness or inflexibility of the muscles. In rigidity, the muscle tone of an affected limb is always stiff and does not relax, sometimes resulting in a decreased range of motion. Rigidity can cause pain and cramping.
(iv) Postural Instability (Impaired Balance and Coordination): Subjects with PD often experience instability when standing, or have impaired balance and coordination. The subject may go through periods of “freezing,” in which the subject finds it difficult to start walking. Slowness and incompleteness of movement can also affect speaking and swallowing.

Not all PD subjects will experience secondary motor symptoms. However, most subjects typically exhibit one or more of the following secondary motor symptoms: stooped posture, a tendency to lean forward, dystonia, fatigue, impaired fine motor dexterity and motor coordination, impaired gross motor coordination, poverty of movement (decreased arm swing), akathisia, speech problems, such as softness of voice or slurred speech caused by lack of muscle control, loss of facial expression, or “masking”, micrographia (small, cramped handwriting), difficulty swallowing, sexual dysfunction, and drooling.

A number of non-motor symptoms are also associated with PD. However, these symptoms are not specific for PD, and are typically only identified as indicating PD retrospectively. That is, the non-motor symptoms experienced by a subject are not typically recognized as indicating PD until after the presence of primary and secondary motor symptoms has been confirmed. Even so, a PD patient will typically exhibit one or more of the following non-motor symptoms: pain, dementia or confusion, sleep disturbances (e.g. REM sleep behavior disorder (RBD)), hyposmia, constipation, skin problems, depression, fear or anxiety, memory difficulties and slowed thinking, urinary problems, fatigue and aching, loss of energy, compulsive behavior (e.g. gambling), and cramping.

The PD subject may be categorized according to the Hoehn-Yahr scale. The Hoehn-Yahr scale is a commonly used system for describing how the symptoms of Parkinson's disease progress. The scale allocates stages from 0 to 5 to indicate the relative level of disability, as follows:
Stage 0: No signs of disease
Stage 1.0: Symptoms are very mild; unilateral involvement only
Stage 1.5: Unilateral and axial involvement
Stage 2: Bilateral involvement without impairment of balance
Stage 2.5: Mild bilateral disease with recovery on pull test
Stage 3: Mild to moderate bilateral disease; some postural instability; physically independent
Stage 4: Severe disability; still able to walk or stand unassisted
Stage 5: Wheelchair bound or bedridden unless aided

The PD subject may have been diagnosed with PD according to the UK Parkinson's Disease Society Brain Bank criteria. These criteria are:

### Step 1: Diagnosis of Parkinsonian Syndrome

Bradykinesia and at least one of the following: muscular rigidity, 4-6 Hz rest tremor, postural instability not caused by primary visual, vestibular, cerebellar, or proprioceptive dysfunction.

### Step 2: Identification of Features Tending to Exclude Parkinson's Disease as the Cause of Parkinsonism

History of repeated strokes with stepwise progression of parkinsonian features
History of repeated head injury
History of definite encephalitis
Oculogyric crises
Neuroleptic treatment at onset of symptoms
More than one affected relative
Sustained remission
Strictly unilateral features after 3 years
Supranuclear gaze palsy
Cerebellar signs
Early severe autonomic involvement
early severe dementia with disturbances of memory, language, and praxis
Babinski sign
presence of cerebral tumor or communication hydrocephalus on imaging study
negative response to large doses of levodopa in absence of malabsorption
MPTP (1-methyl 4-phenyl 1,2,3,6-tetrahydropyridine) exposure
   Step 3: Identification of Features that Support a Diagnosis of Parkinson's Disease (Three or More in Combination with Step 1 Required for Diagnosis of Definite Parkinson's Disease):
Unilateral onset
Rest tremor present
Progressive disorder
Persistent asymmetry affecting side of onset most
Excellent response (70-100%) to levodopa
Severe levodopa-induced chorea
Levodopa response for 5 years or more
Clinical course of ten years or more

The individual may be suspected of being at risk of developing PD because of the presence of one or more factors which increase susceptibility to PD. The individual may have a familial history of PD. Large epidemiological studies demonstrate that people with an affected first-degree relative, such as a parent or sibling, have a two-to-three fold increased risk of developing Parkinson's, as compared to the general population.

The individual may have a mutation or polymorphism in a gene or locus associated with PD. For example, the individual may have a mutation or polymorphism in one of more of the following genes or loci: PARK1 (gene encoding α-synuclein (SNCA)), PARK2 (gene encoding suspected ubiquitin-protein ligase Parkin (PRKN2)), PARK3, PARK4, PARK5 (gene encoding ubiquitin carboxy-terminal hydrolase Li), PARK6 (gene encoding a putative protein kinase (PINK1)), PARK7 (gene encoding DJ-1), or PARK8 (gene encoding leucine-rich repeat kinase 2 (LRRK2)).

The individual may have a mutation or polymorphism in one or more of the genes encoding the following products: Dopamine receptor 2, Dopamine receptor 4, Dopamine transporter, Monoamine oxidase A, Monoamine oxidase B, Catechol-o-methyl-transferase, N-acetyl transferase 2 detoxification enzyme, Apo-lipoprotein E, Glutathione transferase detoxification enzyme T1, Glutathione transferase detoxification enzyme M1, Glutathione transferase detoxification enzyme, or Glutathione transferase detoxification enzyme Z1; and/or in the tRNA Glu mitochondrial gene and/or the Complex 1 mitochondrial gene. Preferably the individual has a mutation or polymorphism in the gene encoding Monoamine oxidase B, and/or N-acetyl transferase 2 detoxification enzyme, and/or Glutathione transferase detoxification enzyme T1 and/or in the tRNA Glu mitochondrial gene.

Environmental risk factors may also be present. To date, epidemiological research has identified rural living, well water, herbicide use and exposure to pesticides as factors that may be linked to PD. Also, MPTP (1-methyl 4-phenyl 1,2,3,6-tetrahydropyridine) can cause Parkinsonism if injected. The chemical structure of MPTP is similar to the widely used herbicide paraquat and damages cells in a way similar to the pesticide rotenone, as well as some other substances.

One or more of the clinical parameters, or symptoms, mentioned above, can be assessed in combination with one or more of NAD, EMA, or the markers set forth in Tables 2 and 5, in order to diagnose PD in a subject.

As used herein, a “reference level” of a marker means a level of the marker that is indicative of a particular disease state, phenotype, or lack thereof, as well as combinations of disease states, phenotypes, or lack thereof. A “positive” reference level of a marker means a level that is indicative of a particular disease state or phenotype. A “negative” reference level of a marker means a level that is indicative of a lack of a particular disease state or phenotype. For example, a “Parkinson's disease-positive reference level” of a marker means a level of a marker that is indicative of a positive diagnosis of Parkinson's disease in a subject, and a “Parkinson's disease-negative reference level” of a marker means a level of a marker that is indicative of a negative diagnosis of Parkinson's disease in a subject. A “reference level” of a marker may be an absolute or relative amount or concentration of the marker, a presence or absence of the marker, a range of amount or concentration of the marker, a minimum and/or maximum amount or concentration of the marker, a mean amount or concentration of the marker, and/or a median amount or concentration of the marker; and, in addition, “reference levels” of combinations of markers may also be ratios of absolute or relative amounts or concentrations of two or more markers with respect to each other. Appropriate positive and negative reference levels of markers for a particular disease state, phenotype, or lack thereof may be determined by measuring levels of desired markers in one or more appropriate subjects, and such reference levels may be tailored to specific populations of subjects (e.g., a reference level may be age-matched so that comparisons may be made between marker levels in samples from subjects of a certain age and reference levels for a particular disease state, phenotype, or lack thereof in a certain age group). Such reference levels may also be tailored to specific techniques that are used to measure levels of markers in biological samples (e.g., LC-MS, GC-MS, etc.), where the levels of markers may differ based on the specific technique that is used.

As used herein, “sample” or “biological sample” includes a specimen or culture obtained from any source. Biological samples can be obtained from blood (including any blood product, such as whole blood, plasma, serum, or specific types of cells of the blood), urine, saliva, and the like. Biological samples also include tissue samples, such as pathological tissues that have previously been fixed (e.g., formaline snap frozen, cytological processing, etc.). In one embodiment, the biological sample is from blood.

As use herein, the phrase “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

The phrase “specific identification” is understood as detection of a marker of interest with sufficiently low background of the assay and cross-reactivity of the reagents used such that the detection method is diagnostically useful. In certain embodiments, reagents for specific identification of a marker bind to only one isoform of the marker. In certain embodiments, reagents for specific identification of a marker bind to more than one isoform of the marker. In certain embodiments, reagents for specific identification of a marker bind to all known isoforms of the marker.

As used herein, the phrase “subject suspected of having Parkinson's disease” refers to a subject that presents one or more symptoms indicative of Parkinson's disease. A subject suspected of having Parkinson's disease may also have one or more risk factors. A subject suspected of having Parkinson's disease has generally not been tested for Parkinson's disease. However, a “subject suspected of having Parkinson's disease” encompasses an individual who has received an initial diagnosis, but for whom the stage of Parkinson's disease is not known.

The term “such as” is used herein to mean, and is used interchangeably, with the phrase “such as but not limited to.”

The term “therapeutic effect” refers to a local or systemic effect in animals, particularly mammals, and more particularly humans caused by a pharmacologically active substance. The term thus means any substance intended for use in the diagnosis, cure, mitigation, treatment, or prevention of disease, or in the enhancement of desirable physical or mental development and conditions in an animal or human. A therapeutic effect can be understood as a decrease in the symptoms of Parkinson's disease such as rest tremor, bradykinesia, rigidity or loss of postural stability.

As used herein, “therapeutically effective amount” means the amount of a compound that, when administered to a patient for treating a disease, is sufficient to effect such treatment for the disease, e.g., the amount of such a substance that produces some desired local or systemic effect at a reasonable benefit/risk ratio applicable to any treatment, e.g., is sufficient to ameliorate at least one sign or symptom of the disease, e.g., to prevent progression of the disease or condition, e.g., rest tremor, bradykinesia, rigidity or loss of postural stability. When administered for preventing a disease, the amount is sufficient to avoid or delay onset of the disease. The “therapeutically effective amount” will vary depending on the compound, its therapeutic index, solubility, the disease and its severity and the age, weight, etc., of the patient to be treated, and the like. For example, certain compounds discovered by the methods of the present invention may be administered in a sufficient amount to produce a reasonable benefit/risk ratio applicable to such treatment. Administration of a therapeutically effective amount of a compound may require the administration of more than one dose of the compound.

As used herein, “treatment,” particularly “active treatment,” refers to performing an intervention to treat Parkinson's disease in a subject, e.g., reduce at least one of rest tremor, bradykinesia, rigidity or loss of postural stability. There is no cure for PD, but medications and surgery can provide relief from the symptoms. Dopamine replacement drugs, dopamine agonists, Catechol-O-methyl transferase (COMT) inhibitors, e.g., Entac, monoamine oxidase B (MAO-B) inhibitors, and Amantadine are examples of drugs used in the treatment of PD.

The dopamine replacement drug Levodopa (L-DOPA, L-3,4-dihydroxyphenylalanine) has been the most widely used treatment for over 30 years. L-DOPA is converted into dopamine in the dopaminergic neurons by dopa decarboxylase. Since motor symptoms are produced by a lack of dopamine in the substantia nigra, the administration of L-DOPA temporarily diminishes the motor symptoms. Levodopa is usually combined with a dopa decarboxylase inhibitor or COMT inhibitor. The other main families of drugs useful for treating motor symptoms are dopamine agonists and monoamine oxidase B (MAO-B) inhibitors such as selegiline and rasagiline. MAO-B breaks down dopamine secreted by the dopaminergic neurons, and MAO-B inhibitors increase the level of dopamine in the basal ganglia by blocking its metabolism. The reduction in MAO-B activity results in increased L-DOPA in the striatum. See, The National Collaborating Centre for Chronic Conditions, ed. (2006), “Symptomatic pharmacological therapy in Parkinson's disease”, Parkinson's Disease. London: Royal College of Physicians. pp. 59-100. Amantadine (Symmetrel™), originally developed as an antiviral drug, is also used to treat PD. Amantadine is the organic compound 1-adamantylamine or 1-aminoadamantane, meaning it consists of an adamantane backbone that has an amino group substituted at one of the four methene positions.

A “transcribed polynucleotide” or “nucleotide transcript” is a polynucleotide (e.g. an mRNA, hnRNA, a cDNA, or an analog of such RNA or cDNA) which is complementary to or having a high percentage of identity (e.g., at least 80% identity) with all or a portion of a mature mRNA made by transcription of a marker of the invention and normal post-transcriptional processing (e.g. splicing), if any, of the RNA transcript, and reverse transcription of the RNA transcript.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

Exemplary compositions and methods of the present invention are described in more detail in the following sections: (C) Biomarkers of the invention; (D) Biological samples; (E) Detection and/or measurement of the biomarkers of the invention; (F) Isolated biomarkers; (G) Applications of biomarkers of the invention; and (H) Kits/panels.

### C. Biomarkers of the Invention

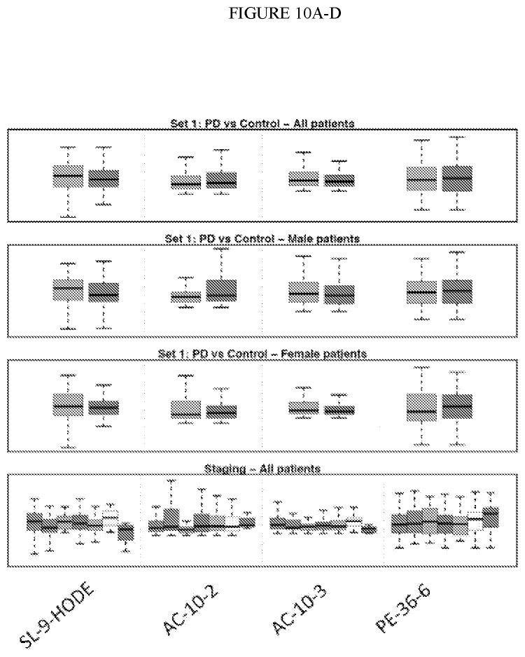

The present invention is based, at least in part, on the discovery that the levels of biomarkers, e.g., protein, metabolite or lipid markers, in Table 2 and Table 5 including NAP and EMA are modulated in Parkinson's disease (see, e.g., FIGS. 10A-C and FIGS. 11A-C). In some embodiments, one or more of NAP and EMA, or one or more of the markers in Table 2 and Table 5 are increased in samples from subjects suffering from Parkinson's disease as compared to a control. In other embodiments, one or more of NAP and EMA, or one or more of the markers in Table 2 and Table 5 are decreased in samples from subjects suffering from Parkinson's disease as compared to a control. Accordingly, the invention provides methods for diagnosing and/or monitoring (e.g., monitoring of disease progression or treatment) and/or prognosing Parkinson's disease, in a mammal.

Moreover, the present invention is based, at least in part, on the discovery that the levels of biomarkers, e.g., protein, metabolite, or lipid markers, in Table 2 and Table 5, including one or more of NAP and EMA, are modulated in various stages of Parkinson's disease (see, e.g, FIG. 10D and FIG. 11D). For example, stages of Parkinson's disease can be based on the Hoehn-Yahr scale, e.g., Hoehn-Yahr scale 0, scale 1, scale 1.5, scale 2, scale 2.5, scale 3, scale 4, or scale 5. In some embodiments, one or more of NAP and EMA, or one or more of the markers in Table 2 and Table 5 are increased as stages of the disease progress in subjects suffering from Parkinson's disease. In other embodiments, one or more of NAP and EMA, or one or more of the markers in Table 2 and Table 5 are decreased as stages of the disease progress in subjects suffering from Parkinson's disease. Accordingly, the invention provides methods for diagnosing the stage of Parkinson's disease in a subject and/or monitoring (e.g., monitoring of disease progression or treatment) and/or prognosing Parkinson's disease, in a mammal, based on the stage of the disease.

The invention also provides methods for treating or for adjusting treatment regimens based on diagnostic information relating to the levels of the markers NAP, EMA, as well as others in Table 2 and Table 5 in a sample, e.g., a plasma, serum, cerebrospinal fluid or urine sample, of a subject with Parkinson's disease. The invention further provides panels and kits for practicing the methods of the invention.

The present invention provides new markers and combinations of markers for use in diagnosing and/or prognosing Parkinson's disease, and in particular, markers for use in diagnosing and/or prognosing Parkinson's disease. The markers of the invention are meant to encompass any measurable characteristic that reflects in a quantitative or qualitative manner the physiological state of an organism, e.g., whether the organism has Parkinson's disease and/or what stage of Parkinson's disease the organism has. The physiological state of an organism is inclusive of any disease or non-disease state, e.g., a subject having Parkinson's disease or a subject who is otherwise healthy. Said another way, the markers of the invention include characteristics that can be objectively measured and evaluated as indicators of normal processes, pathogenic processes, or pharmacologic responses to a therapeutic intervention, including, in particular, Parkinson's disease. Markers can be clinical parameters (e.g., age, performance status such as that in an anxiety test, a sleep test, a smell test, or any combination thereof), laboratory measures (e.g., molecular markers), imaging-based measures, or genetic or other molecular determinants, as well as combinations thereof. Examples of markers include, for example, polypeptides, peptides, polypeptide fragments, proteins, antibodies, hormones, polynucleotides, RNA or RNA fragments, microRNA (miRNAs), lipids (e.g. structural lipids or signaling lipids), polysaccharides, and other bodily metabolites that are diagnostic and/or indicative and/or predictive of a disease, e.g., Parkinson's disease. Examples of markers also include polypeptides, peptides, polypeptide fragments, proteins, antibodies, hormones, polynucleotides, RNA or RNA fragments, microRNA (miRNAs), lipids (e.g. structural lipids or signaling lipids), polysaccharides, and other bodily metabolites diagnostic and/or indicative and/or predictive of any stage or clinical phase of a disease, such as, Parkinson's disease. Clinical stage or phase can be represented by any means known in the art, for example, based on the Hoehn-Yahr scale, e.g., Hoehn-Yahr scale 0, scale 1, scale 1.5, scale 2, scale 2.5, scale 3, scale 4, or scale 5 Parkinson's disease.

In one aspect, the present invention relates to using, measuring, detecting, and the like of one or more of NAP and EMA, or one or more of the markers listed in Table 2 and Table 5 alone, or together with one or more additional markers of Parkinson's disease.

The markers in Table 5 include oxaloacetate/methysuccinate/ethylmalonic acid/glutaric acid, N-acetyl puterscine (NAP), P13591 (NCAM), SL-9-HODE, Q14624.3 (ITIH4), F5GZZ9 (CD163), AC-10:2, AC-10:3, and PE-36:6.

Additional markers listed in Table 2 include 2-ketohexanoic acid, D-erythrose-4-phosphate, kynurenine, methylguanine, 1-methyladenosine, phosphoserine, deoxyinosine, TRIM14 (Tripartite Motif Containing 14), SGK223 (Tyrosine-Protein Kinase SgK223), PROS1 (Protein S (Alpha)), C4BPA (Complement Component 4 Binding Protein Alpha), C4BPB (Complement Component 4 Binding Protein Beta), and HP (Haptoglobin).

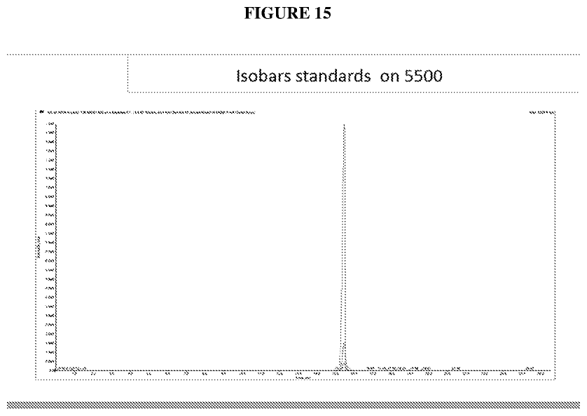

The marker identified as “oxaloacetate/methysuccinate/ethylmalonic acid/glutaric acid” refers to any one or more of oxaloacetate, methysuccinate, ethylmalonic acid, or glutaric acid. It was found that there is no separation between oxaloacetic acid, methylsuccinic acid, ethylmalonic acid and glutaric acid was observed in an HILIC-LS-MS/MS mass chromatogram (see FIG. 15) obtained as described in Example 1, and thus the markers oxaloacetate, methysuccinate, ethylmalonic acid and glutaric acid are indistinguishable using the method of Example 1. Therefore, the marker identified herein as “oxaloacetate/methysuccinate/ethylmalonic acid/glutaric acid” is intended to include any one or more, e.g., one, two, three or all four of the markers oxaloacetic acid (oxaloacetate), methylsuccinic acid (methylsuccinate), ethylmalonic acid and glutaric acid (e.g., each alone or in combination with each other). A new method to separate these four markers was later developed. See Peng et al., 2022, Analytical Biochemistry 645: 114604, which is incorporated by reference herein in its entirety.

In one embodiment, these markers may be detected and used in the methods of the invention separately from each other using methods known in the art. In another embodiment, two, three, or four of these markers may be detected in combination. In a preferred embodiment, methylsuccinate is detected and used in the methods of the invention.

Other markers that may be used in combination with NAP, and/or EMA, specifically, as well as other markers in Table 2 and Table 5, include any measurable characteristic described herein that reflects in a quantitative or qualitative manner the physiological state of an organism, e.g., whether the organism has Parkinson's disease and/or what stage of Parkinson's disease the organism has. The physiological state of an organism is inclusive of any disease or non-disease state, e.g., a subject having Parkinson's disease or a subject who is otherwise healthy. The markers of the invention that may be used in combination with the markers in Table 2 and Table 5 include characteristics that can be objectively measured and evaluated as indicators of normal processes, pathogenic processes, or pharmacologic responses to a therapeutic intervention, including, in particular, Parkinson's disease. Such combination markers can be clinical parameters (e.g., age, performance status such as that in an anxiety test, a sleep test, a smell test, or any combination thereof), laboratory measures (e.g., molecular markers), imaging-based measures, or genetic or other molecular determinants. Examples of markers for use in combination with one or more of NAP and EMA, or one or more of the markers in Table 2 and Table 5 include, for example, polypeptides, peptides, polypeptide fragments, proteins, antibodies, hormones, polynucleotides, RNA or RNA fragments, microRNA (miRNAs), lipids, polysaccharides, and other bodily metabolites that are diagnostic and/or indicative and/or predictive of Parkinson's disease, or any particular stage or phase of Parkinson's disease, e.g., Hoehn-Yahr scale 0, scale 1, scale 1.5, scale 2, scale 2.5, scale 3, scale 4, or scale 5 Parkinson's disease. In other embodiments, the present invention also involves the analysis and consideration of any clinical and/or patient-related health data, for example, data obtained from an Electronic Medical Record (e.g., collection of electronic health information about individual patients or populations relating to various types of data, such as, demographics, medical history, medication and allergies, immunization status, laboratory test results, radiology images, vital signs, personal statistics like age and weight, and billing information).

The present invention also contemplates the use of one or more of NAP and EMA, or one or more of the markers listed in Table 2 and Table 5, i.e., oxaloacetate/methysuccinate/ethylmalonic acid/glutaric acid, N-acetyl puterscine, P13591 (NCAM), SL-9-HODE, Q14624.3 (MH4), F5GZZ9 (CD163), AC-10:2, AC-10:3, and PE-36:6 or Table 2, i.e., 2-ketohexanoic acid, D-erythrose-4-phosphate, kynurenine, methylguanine, 1-methyladenosine, phosphoserine, deoxyinosine, TRIM14 (Tripartite Motif Containing 14), SGK223 (Tyrosine-Protein Kinase SgK223), PROS1 (Protein S (Alpha)), C4BPA (Complement Component 4 Binding Protein Alpha), C4BPB (Complement Component 4 Binding Protein Beta), and HP (Haptoglobin).

In one embodiment, the invention contemplates marker sets with at least two (2) members, which may include NAP and EMA, or any two of the markers in Table 2 and Table 5. In another embodiment, the invention contemplates marker sets with at least three (3) members, which may include any three of the markers in Table 2 and Table 5. In another embodiment, the invention contemplates marker sets with at least four (4) members, which may include any four of the markers in Table 2 and Table 5. In another embodiment, the invention contemplates marker sets with at least five (5) members, which may include any five of the markers in Table 2 and Table 5. In another embodiment, the invention contemplates marker sets with at least six (6) members, which may include any six of the markers in Table 2 and Table 5. In another embodiment, the invention contemplates marker sets with at least seven (7) members, which may include any seven of the markers in Table 2 and Table 5. In another embodiment, the invention contemplates marker sets with at least eight (8) members, which may include any eight of the markers in Table 2 and Table 5. In another embodiment, the invention contemplates marker sets with at least nine (9) members, which may include any nine of the markers in Table 2 and Table 5. In another embodiment, the invention contemplates marker sets with at least nine (9) members, which may include any nine of the markers in Table 2 and Table 5. In another embodiment, the invention contemplates marker sets with at least ten (10) members, which may include any ten of the markers in Table 2 and Table 5. In another embodiment, the invention contemplates marker sets with at least eleven (11) members, which may include any eleven of the markers in Table 2 and Table 5. In another embodiment, the invention contemplates marker sets with at least twelve (12) members, which may include any twelve of the markers in Table 2 and Table 5. In another embodiment, the invention contemplates marker sets with at least thirteen (13) members, which may include any thirteen of the markers in Table 2 and Table 5. In another embodiment, the invention contemplates marker sets with at least fourteen (14) members, which may include any fourteen of the markers in Table 2 and Table 5. In another embodiment, the invention contemplates marker sets with at least fifteen (15) members, which may include any fifteen of the markers in Table 2 and Table 5. In another embodiment, the invention contemplates marker sets with at least sixteen (16) members, which may include any sixteen of the markers in Table 2 and Table 5. In another embodiment, the invention contemplates marker sets with at least seventeen (17) members, which may include any seventeen of the markers in Table 2 and Table 5. In another embodiment, the invention contemplates marker sets with at least eighteen (18) members, which may include any eighteen of the markers in Table 2 and Table 5. In another embodiment, the invention contemplates marker sets with at least nineteen (19) members, which may include any nineteen of the markers in Table 2 and Table 5. In another embodiment, the invention contemplates marker sets with at least twenty (20) members, which may include any twenty of the markers in Table 2 and Table 5. In another embodiment, the invention contemplates marker sets with at least twenty-one (21) members, which may include any twenty-one of the markers in Table 2 and Table 5. In another embodiment, the invention contemplates marker sets with at least twenty-two (22) members, which may include any twenty-two of the markers in Table 2 and Table 5. In other embodiments, the invention contemplates a marker set comprising at least 1, 2, 3, 4, 5, 6, 7, 8, 9, 10, 11, 12, 13, 14, 15, 16, 17, 18, 19, 20, 21, 22, 23, 24, or 25 of the markers listed in Table 2 and Table 5.

In certain embodiments, one or more of NAP and EMA, or one or more of the markers in Table 2 and Table 5 may be used in combination with at least one other marker, or more preferably, with at least two other markers, or still more preferably, with at least three other markers, or even more preferably with at least four other markers. Still further, one or more of NAP and EMA, or one or more of the markers in Table 2 and Table 5 in certain embodiments, may be used in combination with at least five other markers, or at least six other markers, or at least seven other markers, or at least eight other markers, or at least nine other markers, or at least ten other markers, or at least eleven other markers, or at least twelve other markers, or at least thirteen other markers, or at least fourteen other markers, or at least fifteen other markers, or at least sixteen other markers, or at least seventeen other markers, or at least eighteen other markers, or at least nineteen other markers, at least twenty other markers, or at least twenty-one other markers. Further, one or more of NAP and EMA, or one or more of the markers in Table 2 and Table 5 may be used in combination with a multitude of other markers, including, for example, with between about 20-50 other markers, or between 50-100, or between 100-500, or between 500-1000, or between 1000-10,000 markers or more.

In certain embodiments, the present invention contemplates the detection and/or analysis of N-acetyl putrescine (NAP), oxaloacetate, methysuccinate, ethylmalonic acid (EMA), and/or glutaric acid, alone or in combination with any one or more of the following set of biomarkers from Tables 2 and 5: P13591 (NCAM), SL-9-HODE, N-acetylputerscine, Q14624.3 (ITIH4), F5GZZ9 (CD163), AC-10:2, AC-10:3, PE-36:6, 2-ketohexanoic acid, D-erythrose-4-phosphate, kynurenine, methylguanine, 1-methyladenosine, phosphoserine, deoxyinosine, TRIM14 (Tripartite Motif Containing 14), SGK223 (Tyrosine-Protein Kinase SgK223), PROS1 (Protein S (Alpha)), C4BPA (Complement Component 4 Binding Protein Alpha), C4BPB (Complement Component 4 Binding Protein Beta), and HP (Haptoglobin). For example, the present invention contemplates the detection and/or analysis of the combination of NAP and EMA. In one embodiment, an increase in the level of one or more of markers oxaloacetate, methysuccinate, ethylmalonic acid (EMA), N-acetyl putrescine (NAP), and/or glutaric acid as compared to a control, indicates that the subject has Parkinson's disease. In another embodiment, a level of one or more of markers oxaloacetate, methysuccinate, ethylmalonic acid (EMA), N-acetyl putrescine (NAP), and/or glutaric acid above a predetermined threshold, indicates that the subject has Parkinson's disease.

In other embodiments, the present invention contemplates the detection and/or analysis of BM000397 (N-acetylputerscine (NAP)), alone or in combination with ethylmalonic acid (EMA) or any one or more of the following set of biomarkers from Tables 2 and 5: P13591 (NCAM), SL-9-HODE, oxaloacetate/methysuccinate/ethylmalonic acid/glutaric acid, Q14624.3 (ITIH4), F5GZZ9 (CD163), AC-10:2, AC-10:3, PE-36:6, 2-ketohexanoic acid, D-erythrose-4-phosphate, kynurenine, methylguanine, 1-methyladenosine, phosphoserine, deoxyinosine, TRIM14 (Tripartite Motif Containing 14), SGK223 (Tyrosine-Protein Kinase SgK223), PROS1 (Protein S (Alpha)), C4BPA (Complement Component 4 Binding Protein Alpha), C4BPB (Complement Component 4 Binding Protein Beta), and HP (Haptoglobin). In one embodiment, an increase in the level of marker N-acetylputerscine (NAP) as compared to a control, indicates that the subject has Parkinson's disease.

In another embodiment, a level of N-acetyl puterscine (NAP) above a predetermined level, indicates that the subject has Parkinson's disease.

In other embodiments, the present invention contemplates the detection and/or analysis of each of the markers in Table 5, for use in the methods of the invention.

In other embodiments, the present invention contemplates the detection and/or analysis of BM000397 (N-acetyl puterscine), methylsuccinate, and SL-9-HODE, for use in the methods of the invention.

In still other embodiments, the present invention contemplates the detection and/or analysis of BM000397 (N-acetyl puterscine) and methylsuccinate for use in the methods of the invention.

In another embodiment, a biomarker of the invention is one that is metabolically stable over time (e.g., over the course of 1, 2, 3, 4, 5, 6, 7, or more days), and is metabolically stable regardless of the diet or circadian rhythm of the subject. In still another embodiment, a biomarker of the invention is one that has a consistent biomarker profile regardless of whether or not the patient had been previously or is currently taking medications for PD or a related disease or disorder.

In certain embodiments, the marker is a protein, for example, a protein listed in Table 2 and Table 5. In certain embodiments, the marker is a metabolite or lipid, for example, a metabolite or lipid listed in Table 2 or Table 5. In some embodiments, the invention also relates to a marker set comprising one or more of the markers listed in Table 2 and Table 5. In other embodiments the marker is a nucleic acid, for example, a nucleic acid encoding a protein listed in Table 2 and Table 5.

The markers may also be combined in a marker set comprising at least 1, 2, 3, 4, 5, 6, 7, 8, 9, 10, 11, 12, 13, 14, 15, 16, 17, 18, 19, 20, 21, 22, 23, 24, or 25 of the markers listed in Table 2 and Table 5.

While not wishing to be bound by theory, a brief exemplary description of the biomarkers of Table 5, which are known in the art, is provided as follows:
Oxaloacetate, also referred to as oxaloacetic acid, is a dicarboxylic acid ketone that is an important metabolic intermediate of the citric acid cycle, and has a molecular formula C₄H₄O₅ and a molecular weight of 132.071.
Methysuccinate, also referred to as methylsuccinic acid, is a metabolite found in human fluids. Increased urinary levels of methylsuccinic acid (together with ethylmalonic acid) are the main biochemical measurable features in ethylmalonic encephalopathy. Methylsuccinic acid has a molecular formula of C₅H₈O₄ and a molecular weight of 132.11.
Ethylmalonic acid (EMA) is a branched fatty acid having a molecular formula of C₅H₈O₄ and a molecular weight of 132.11.
Glutaric acid has a molecular formula of C₅H₈O₄ or COOH(CH₂)₃COOH and a molecular weight of 132.11.
N-acetyl puterscine (NAP) is an N-monoacetylalkane-α,ω-diamine that is the N-monoacetyl derivative of putrescine, having the molecular formula C₆—H₁₄—N₂—O, and a molecular weight of 130.19.
NCAM (Neural Cell Adhesion Molecule 1) is a cell adhesion protein, which is a member of the immunoglobulin superfamily. NCAM is a protein having 858 amino acids, with an amino acid sequence set forth in GenBank Accession No. NP_851996. NCAM is identified by Uniprot identification number P13591.
ITIH4 (Inter-alpha-trypsin inhibitor heavy chain H4) is a Type II acute-phase protein (APP) involved in inflammatory responses to trauma, which is identified by Uniprot identification number Q14624.3 and has an amino acid sequence set forth in GenBank Accession No. NP_002209.
CD163 (Scavenger receptor cysteine-rich type I protein M130) is a member of the scavenger receptor cysteine-rich (SRCR) superfamily, and is expressed in monocytes and macrophages. CD163 is has a Uniprot identification number F5GZZ9 and has an amino acid sequence set forth in GenBank Accession No. NP_004235.
SL-9-HODE is a fatty acid resulting from the non-enzymatic oxidation of linoleic acid.
AC-10:2 is a fatty acid having 10 carbon atoms and 2 unsaturated linkages. AC-10:3 is a fatty acid having 10 carbon atoms and 3 unsaturated linkages.
PE-36:6 is a phosphatidylethanolamine structural lipid having 36 carbon atoms and 6 unsaturated linkages.

While not wishing to be bound by theory, a brief exemplary description of the biomarkers of Table 2 (which are not included in Table 5), and which are known in the art, is provided as follows:
2-ketohexanoic acid is an insulin secretagogue having the molecular formula C₆H₁₀O₃ and a molecular weight of 130.1.
D-erythrose-4-phosphate is a phosphate of the simple sugar erythrose having the molecular formula C₄H₉O₇P and a molecular weight of 200.08.
Kynurenine is a metabolite of the amino acid tryptophan used in the production of niacin having the molecular formula C₁₀H₁₂N₂O₃ and a molecular weight of 208.2.
Methylguanine is a derivative of the nucleobase guanine in which a methyl group is attached to the oxygen atom.
1-methyladenosine is one of the modified nucleosides, the levels of which are elevated in urine of patients with malignant tumors. 1-methyladenosine has the molecular formula C₁₁H₁₅N₅O₄ and a molecular weight of 281.2.
Phosphoserine has the molecular formula C₃H₈NO₆P and a molecular weight of 185.07.
Deoxyinosine is a nucleoside that is formed when hypoxanthine is attached to a deoxyribose ring (also known as a ribofuranose) via a beta-N9-glycosidic bond. Deoxyinosine is found in DNA while Inosine is found in RNA. Deoxyinosine has the molecular formula C₁₀H₁₂N₄O₄ and a molecular weight of 252.2.
TRIM4 (Tripartite Motif Containing 14) has a Uniprot identification number Q14142 and an amino acid sequence set forth in GenBank Accession No. AAH06333.
SGK223 (Tyrosine-Protein Kinase SgK223) has a Uniprot identification number Q86YV5 and an amino acid sequence set forth in GenBank Accession No. NP_001074295.
PROS) (Vitamin K dependent Protein S) has a Uniprot identification number P07225 and an amino acid sequence set forth in GenBank Accession No. NP_001301006.
C4BPA (Complement Component 4 Binding Protein Alpha) has a Uniprot identification number P04003 and an amino acid sequence set forth in GenBank Accession No. AAH22312.
C4BPB (Complement Component 4 Binding Protein Beta) has a Uniprot identification number P20851 and an amino acid sequence set forth in GenBank Accession No. AAH05378.
HP (Haptoglobin) has a Uniprot identification number P00738 and an amino acid sequence set forth in GenBank Accession No. AAA88080.

In another aspect, the present invention provides for the identification of a “diagnostic signature” or “disease profile” based on the levels of the markers of the invention in a biological sample, including in a diseased tissue or directly from the serum or blood, that correlates with the stage, presence and/or risk and/or prognosis of Parkinson's disease. The “levels of the markers” can refer to the level of a marker lipid, protein, or metabolite in a biological sample, e.g., plasma or serum. The “levels of the markers” can also refer to the expression level of the genes corresponding to the proteins, e.g., by measuring the expression levels of the corresponding marker mRNAs. The collection or totality of levels of markers provides a diagnostic signature that correlates with the presence and/or stage and/or diagnosis and/or progression of Parkinson's disease. The methods for obtaining a diagnostic signature or disease profile of the invention are meant to encompass any measurable characteristic that reflects in a quantitative or qualitative manner the physiological state of an organism, e.g., whether the organism has Parkinson's disease and/or what stage of Parkinson's disease the organism has. The physiological state of an organism is inclusive of any disease or non-disease state, e.g., a subject having Parkinson's disease or a subject who is otherwise healthy. Said another way, the methods used for identifying a diagnostic signature or disease profile of the invention include determining characteristics that can be objectively measured and evaluated as indicators of normal processes, pathogenic processes, or pharmacologic responses to a therapeutic intervention, including, in particular, Parkinson's disease. These characteristics can be clinical parameters (e.g., age, performance status), laboratory measures (e.g., molecular markers, such as proteins, lipids, or metabolites), imaging-based measures, or genetic or other molecular determinants. Examples of markers include, for example, polypeptides, peptides, polypeptide fragments, proteins, antibodies, hormones, polynucleotides, RNA or RNA fragments, microRNA (miRNAs), lipids, polysaccharides, and other metabolites that are diagnostic and/or indicative and/or predictive of Parkinson's disease. Examples of markers also include polypeptides, peptides, polypeptide fragments, proteins, antibodies, hormones, polynucleotides, RNA or RNA fragments, microRNA (miRNAs), lipids, polysaccharides, and other metabolites which are diagnostic and/or indicative and/or predictive of any stage or clinical phase of Parkinson's disease, e.g., Hoehn-Yahr scale 0, scale 1, scale 1.5, scale 2, scale 2.5, scale 3, scale 4, or scale 5 Parkinson's disease.

In a particular embodiment, a Parkinson's disease profile or diagnostic signature is determined on the basis of one or more of NAP and EMA, or one or more of the combination of one or more of the markers in Table 2 and/or Table 5 together with one or more additional markers of Parkinson's disease. Other markers that may be used in combination with one or more of NAP and EMA, or one or more of the markers in Table 2 and/or Table 5 include any measurable characteristic that reflects in a quantitative or qualitative manner the physiological state of an organism, e.g., whether the organism has Parkinson's disease and/or what stage of Parkinson's disease the organism has. The physiological state of an organism is inclusive of any disease or non-disease state, e.g., a subject having Parkinson's disease or a subject who is otherwise healthy. Said another way, the markers of the invention that may be used in combination with one or more of NAP and EMA, or one or more of the markers in Table 2 and/or Table 5 include characteristics that can be objectively measured and evaluated as indicators of normal processes, pathogenic processes, or pharmacologic responses to a therapeutic intervention, including, in particular, Parkinson's disease. Such combination markers can be clinical parameters (e.g., age, performance status such as that in an anxiety test, a sleep test, a smell test, or any combination thereof), laboratory measures (e.g., molecular markers), imaging-based measures, or genetic or other molecular determinants. Example of markers for use in combination with one or more of NAP and EMA, or one or more of the markers in Table 2 and Table 5 include, for example, polypeptides, peptides, polypeptide fragments, proteins, antibodies, hormones, polynucleotides, RNA or RNA fragments, microRNA (miRNAs), lipids, polysaccharides, and other metabolites that are diagnostic and/or indicative and/or predictive of Parkinson's disease, or any particular stage or phase of Parkinson's disease, e.g., Hoehn-Yahr scale 0, scale 1, scale 1.5, scale 2, scale 2.5, scale 3, scale 4, or scale 5 Parkinson's disease. In certain embodiments, markers for use in combination with the markers in Table 2 and Table 5 include polypeptides, peptides, polypeptide fragments, proteins, antibodies, hormones, polynucleotides, RNA or RNA fragments, microRNA (miRNAs), lipids, polysaccharides, and other bodily metabolites which are diagnostic and/or indicative and/or predictive of Parkinson's disease, or any stage or clinical phase thereof, Hoehn-Yahr scale 0, scale 1, scale 1.5, scale 2, scale 2.5, scale 3, scale 4, or scale 5 Parkinson's disease. In other embodiments, the present invention also involves the analysis and consideration of any clinical parameters and/or patient-related health data, for example, data obtained from an Electronic Medical Record (e.g., collection of electronic health information about individual patients or populations relating to various types of data, such as, demographics, medical history, medication and allergies, immunization status, laboratory test results, radiology images, vital signs, personal statistics like age and weight, and billing information).

In certain embodiments, the diagnostic signature is obtained by (1) detecting the level(s) of one or more of NAP and EMA, or one or more of the markers in Table 2 and Table 5 in a biological sample, (2) comparing the level(s) of one or more of NAP and EMA, or one or more of the markers in Table 2 and Table 5 to the level(s) of the same marker(s) from a control sample, and (3) detecting if the level(s) of one or more of NAP and EMA, or one or more of the markers in Table 2 and Table 5 is above or below a certain threshold level. If the level(s) of NAP, EMA, or that of the one or more marker in Table 2 and Table 5 is above or below the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the level(s) of the one or more of NAP and EMA, or one or more of markers in Table 2 and Table 5.

In certain other embodiments, the diagnostic signature is obtained by (1) detecting the level of at least two markers in Table 2 and Table 5 in a biological sample, (2) comparing the levels of the at least two markers in Table 2 and Table 5 to the levels of the same markers from a control sample, and (3) determining if the at least two markers in Table 2 and Table 5 detected in the biological sample are above or below a certain threshold level. If the at least two markers in Table 2 and Table 5 are above or below the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the levels of the at least two markers in Table 2 and Table 5. In one embodiment, both of the markers are from Table 5.

In certain other embodiments, the diagnostic signature is obtained by (1) detecting the level of at least three markers in Table 2 and Table 5 in a biological sample, (2) comparing the levels of the at least three markers in Table 2 and Table 5 to the levels of the same markers from a control sample, and (3) determining if the at least three markers in Table 2 and Table 5 detected in the biological sample are above or below a certain threshold level. If the at least three markers in Table 2 and Table 5 are above the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the levels of the at least three markers in Table 2 and Table 5. In one embodiment, each of the three markers are from Table 5.

In certain other embodiments, the diagnostic signature is obtained by (1) detecting the level of at least four markers in Table 2 and Table 5 in a biological sample, (2) comparing the levels of the at least four markers in Table 2 and Table 5 to the levels of the same markers from a control sample, and (3) determining if the at least four markers in Table 2 and Table 5 detected in the biological sample are above or below a certain threshold level. If the at least four markers in Table 2 and Table 5 are above the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the levels of the at least four markers in Table 2 and Table 5. In one embodiment, each of the four markers are from Table 5.

In certain other embodiments, the diagnostic signature is obtained by (1) detecting the level of at least five markers in Table 2 and Table 5 in a biological sample, (2) comparing the levels of the at least five markers in Table 2 and Table 5 to the levels of the same markers from a control sample, and (3) determining if the at least five markers in Table 2 and Table 5 detected in the biological sample are above or below a certain threshold level. If the at least five markers in Table 2 and Table 5 are above the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the levels of the at least five markers in Table 2 and Table 5. In one embodiment, each of the five markers are from Table 5.

In certain other embodiments, the diagnostic signature is obtained by (1) detecting the level of at least six markers in Table 2 and Table 5 in a biological sample, (2) comparing the levels of the at least six markers in Table 2 and Table 5 to the levels of the same markers from a control sample, and (3) determining if the at least six markers in Table 2 and Table 5 detected in the biological sample are above or below a certain threshold level. If the at least six markers in Table 2 and Table 5 are above the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the levels of the at least six markers in Table 2 and Table 5. In one embodiment, each of the six markers are from Table 5.

In certain other embodiments, the diagnostic signature is obtained by (1) detecting the level of at least seven markers in Table 2 and Table 5 in a biological sample, (2) comparing the levels of the at least seven markers in Table 2 and Table 5 to the levels of the same markers from a control sample, and (3) determining if the at least seven markers in Table 2 and Table 5 detected in the biological sample are above or below a certain threshold level. If the at least seven markers in Table 2 and Table 5 are above the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the levels of the at least seven markers in Table 2 and Table 5. In one embodiment, each of the seven markers are from Table 5.

In certain other embodiments, the diagnostic signature is obtained by (1) detecting the level of at least eight markers in Table 2 and Table 5 in a biological sample, (2) comparing the levels of the at least eight markers in Table 2 and Table 5 to the levels of the same markers from a control sample, and (3) determining if the at least eight markers in Table 2 and Table 5 detected in the biological sample are above or below a certain threshold level. If the at least eight markers in Table 2 and Table 5 are above the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the levels of the at least eight markers in Table 2 and Table 5. In one embodiment, each of the eight markers are from Table 5.

In certain other embodiments, the diagnostic signature is obtained by (1) detecting the level of at least nine markers in Table 2 and Table 5 in a biological sample, (2) comparing the levels of the at least nine markers in Table 2 and Table 5 to the levels of the same markers from a control sample, and (3) determining if the at least nine markers in Table 2 and Table 5 detected in the biological sample are above or below a certain threshold level. If the at least nine markers in Table 2 and Table 5 are above the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the levels of the at least nine markers in Table 2 and Table 5. In one embodiment, each of the nine markers are from Table 5.

In certain other embodiments, the diagnostic signature is obtained by (1) detecting the level of at least ten, 11, 12, 13, 14, 15, 16, 17, 18, 19, 20, 21, 22, 23, 24, or 25 markers in Table 2 and Table 5 in a biological sample, (2) comparing the levels of the at least ten, 11, 12, 13, 14, 15, 16, 17, 18, 19, 20, 21, 22, 23, 24, or 25 markers in Table 2 and Table 5 to the levels of the same markers from a control sample, and (3) determining if the at least ten, 11, 12, 13, 14, 15, 16, 17, 18, 19, 20, 21, 22, 23, 24, or 25 markers in Table 2 and Table 5 detected in the biological sample are above or below a certain threshold level. If the at least ten, 11, 12, 13, 14, 15, 16, 17, 18, 19, 20, 21, 22, 23, 24, or 25 markers in Table 2 and Table 5 are above the threshold level, then the diagnostic signature is indicative of Parkinson's disease in the biological sample and/or a particular stage of Parkinson's disease. In certain embodiments, the diagnostic signature can be determined based on an algorithm or computer program that predicts whether the biological sample is from a subject with Parkinson's disease and/or the stage of Parkinson's disease based on the levels of the at least ten, 11, 12, 13, 14, 15, 16, 17, 18, 19, 20, 21, 22, 23, 24, or 25 markers in Table 2 and Table 5.

In accordance with various embodiments, algorithms may be employed to predict whether or not a biological sample is likely to be diseased, e.g., have Parkinson's disease. The skilled artisan will appreciate that an algorithm can be any computation, formula, statistical survey, nomogram, look-up table, decision tree method, or computer program which processes a set of input variables (e.g., number of markers (n) which have been detected at a level exceeding some threshold level, or number of markers (n) which have been detected at a level below some threshold level) through a number of well-defined successive steps to eventually produce a score or “output,” e.g., a diagnosis of Parkinson's disease. Any suitable algorithm—whether computer-based or manual-based (e.g., look-up table)—is contemplated herein.

In certain embodiments, an algorithm of the invention is used to predict whether a biological sample is from a subject that has Parkinson's disease by producing a score on the basis of the detected level of at least 1, 2, 3, 4, 5, 6, 7, 8, 9, 10, 11, 12, 13, 14, 15, 16, 17, 18, 19, 20, 21, 22 of the markers in Table 2 and Table 5 in the sample, wherein if the score is above or below a certain threshold score, then the biological sample is from a subject that has Parkinson's disease.

In other embodiments, an algorithm of the invention is used to predict whether a biological sample is from a subject that his suffering from a certain stage of Parkinson's disease by producing a score on the basis of the detected level of at least 1, 2, 3, 4, 5, 6, 7, 8, 9, 10, 11, 12, 13, 14, 15, 16, 17, 18, 19, 20, 21, 22 of the markers in Table 2 and Table 5 in the sample, wherein if the score is above or below a certain threshold score, then the biological sample is from a subject that is suffering from a certain stage of Parkinson's disease.

Moreover, a Parkinson's disease profile or signature may be obtained by detecting at least one of the markers in Table 2 and Table 5 in combination with at least one other marker, or more preferably, with at least two other markers, or still more preferably, with at least three other markers, or even more preferably with at least four other markers. Still further, the markers in Table 2 and Table 5 in certain embodiments, may be used in combination with at least five other markers, or at least six other markers, or at least seven other markers, or at least eight other markers, or at least nine other markers, or at least ten other markers, or at least eleven other markers, or at least twelve other markers, or at least thirteen other markers, or at least fourteen other markers, or at least fifteen other markers, or at least sixteen other markers, or at least seventeen other markers, or at least eighteen other markers, or at least nineteen other markers, or at least twenty other markers. Further still, the markers in Table 2 and Table 5 may be used in combination with a multitude of other markers, including, for example, with between about 20-50 other markers, or between 50-100, or between 100-500, or between 500-1000, or between 1000-10,000 or markers or more.

In certain embodiments, the markers of the invention can include variant sequences. More particularly, the binding agents/reagents used for detecting the markers of the invention can bind and/or identify variants of the markers of the invention. As used herein, the term “variant” encompasses nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 80%, 85%, 90%, 95%, 96%, 97%, 98% or 99% identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100.

In addition to exhibiting the recited level of sequence identity, variants of the disclosed polypeptide markers are preferably themselves expressed in subjects with Parkinson's disease at levels that are higher or lower than the levels of expression in normal, healthy individuals.

Variant sequences generally differ from the specifically identified sequence only by conservative substitutions, deletions or modifications. As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptide and polynucleotide sequences may be aligned, and percentages of identical amino acids or nucleotides in a specified region may be determined against another polypeptide or polynucleotide sequence, using computer algorithms that are publicly available. The percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity.

Two exemplary algorithms for aligning and identifying the identity of polynucleotide sequences are the BLASTN and FASTA algorithms. The alignment and identity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia, Charlottesville, Va. 22906-9025. The FASTA algorithm, set to the default parameters described in the documentation and distributed with the algorithm, may be used in the determination of polynucleotide variants. The readme files for FASTA and FASTX Version 2.0x that are distributed with the algorithms describe the use of the algorithms and describe the default parameters.

The BLASTN software is available on the NCBI anonymous FTP server and is available from the National Center for Biotechnology Information (NCBI), National Library of Medicine, Building 38A, Room 8N805, Bethesda, Md. 20894. The BLASTN algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTN, is described at NCBI's website and in the publication of Altschul, et al., “Gapped BLAST and PSI-BLAST: a new generation of protein database search programs,” Nucleic Acids Res. 25:3389-3402, 1997.

In an alternative embodiment, variant polypeptides are encoded by polynucleotide sequences that hybridize to a disclosed polynucleotide under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

### D. Biological Samples

The present invention may be practiced with any suitable biological sample that potentially contains, expresses, or includes a detectable disease marker, e.g., a polypeptide marker, or a nucleic acid marker. For example, the biological sample may be obtained from sources that include whole blood, serum, plasma or diseased or healthy tissue. The methods of the invention may especially be applied to plasma. In another embodiment, the present invention may be practiced with any suitable plasma samples which are freshly isolated or which have been frozen or stored after having been collected from a subject, or archival plasma samples, for example, with known diagnosis, treatment and/or outcome history. The methods of the invention may also be applied to urine or cerebrospinal fluid.

The inventive methods may be performed at the single cell level (e.g., isolation and testing of a blood cell). However, preferably, the inventive methods are performed using a sample comprising many cells, where the assay is “averaging” the level of the marker over the entire sample, for example over the collection of cells or tissue present in the sample. Preferably, there is enough of the biological sample to accurately and reliably determine the levels of the marker. In certain embodiments, multiple samples may be taken from the same subject in order to obtain a representative sampling of the subject. In addition, sufficient biological material can be obtained in order to perform duplicate, triplicate or further rounds of testing.

Any commercial device or system for isolating and/or obtaining blood or other biological products, and/or for processing said materials prior to conducting a detection reaction is contemplated.

In certain embodiments, the present invention relates to detecting marker nucleic acid molecules (e.g., mRNA encoding the protein markers in Table 2 and Table 5). In such embodiments, RNA can be extracted from a biological sample, e.g., a blood sample, before analysis. Methods of RNA extraction are well known in the art (see, for example, J. Sambrook et al., “Molecular Cloning: A Laboratory Manual”, 1989, 2^{nd} Ed., Cold Spring Harbour Laboratory Press: New York). Most methods of RNA isolation from bodily fluids or tissues are based on the disruption of the tissue in the presence of protein denaturants to quickly and effectively inactivate RNases. Generally, RNA isolation reagents comprise, among other components, guanidinium thiocyanate and/or beta-mercaptoethanol, which are known to act as RNase inhibitors. Isolated total RNA is then further purified from the protein contaminants and concentrated by selective ethanol precipitations, phenol/chloroform extractions followed by isopropanol precipitation (see, for example, P. Chomczynski and N. Sacchi, Anal. Biochem., 1987, 162: 156-159) or cesium chloride, lithium chloride or cesium trifluoroacetate gradient centrifugations.

Numerous different and versatile kits can be used to extract RNA (i.e., total RNA or mRNA) from bodily fluids or tissues (e.g., blood) and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Qiagen, Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

In certain embodiments, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2^{nd} Ed., Cold Spring Harbour Laboratory Press: New York; “Short Protocols in Molecular Biology”, F. M. Ausubel (Ed.), 2002, 5.sup.th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase).

In certain embodiments, the RNA isolated from the biological sample (for example, after amplification and/or conversion to cDNA or cRNA) is labeled with a detectable agent before being analyzed. The role of a detectable agent is to facilitate detection of RNA or to allow visualization of hybridized nucleic acid fragments (e.g., nucleic acid fragments hybridized to genetic probes in an array-based assay). Preferably, the detectable agent is selected such that it generates a signal which can be measured and whose intensity is related to the amount of labeled nucleic acids present in the sample being analyzed. In array-based analysis methods, the detectable agent is also preferably selected such that it generates a localized signal, thereby allowing spatial resolution of the signal from each spot on the array.

Methods for labeling nucleic acid molecules are well-known in the art. For a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes (see, for example, L. M. Smith et al., Nucl. Acids Res. 1985, 13: 2399-2412) or of enzymes (see, for example, B. A. Connoly and P. Rider, Nucl. Acids. Res. 1985, 13: 4485-4502); chemical modifications of nucleic acid fragments making them detectable immunochemically or by other affinity reactions (see, for example, T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

However, in some embodiments, the expression levels are determined by detecting the expression of a gene product (e.g., protein) thereby eliminating the need to obtain a genetic sample (e.g., RNA) from the biological sample.

In still other embodiments, the present invention relates to preparing a prediction model for the likelihood of progression of Parkinson's disease by preparing a model for Parkinson's disease based on measuring the markers in Table 2 and Table 5 of the invention in known control samples.

The invention further relates to the preparation of a model for Parkinson's disease by evaluating the markers of the invention in known samples of Parkinson's disease. More particularly, the present invention relates to a Parkinson's disease model for diagnosing and/or monitoring and/or prognosing Parkinson's disease using the markers of the invention, which can include the markers in Table 2 and Table 5.

In the methods of the invention aimed at preparing a model for Parkinson's disease, it is understood that the particular clinical outcome associated with each sample contributing to the model preferably should be known. Consequently, the model can be established using archived biological samples. In the methods of the invention aimed at preparing a model for Parkinson's disease, total RNA can be generally extracted from the source material of interest, generally an archived tissue such as a formalin-fixed, paraffin-embedded tissue, and subsequently purified. Methods for obtaining robust and reproducible gene expression patterns from archived tissues, including formalin-fixed, paraffin-embedded (FFPE) tissues are taught in U.S. Publ. No. 2004/0259105, which is incorporated herein by reference in its entirety. Commercial kits and protocols for RNA extraction from FFPE tissues are available including, for example, ROCHE High Pure RNA Paraffin Kit (Roche) MasterPure™ Complete DNA and RNA Purification Kit (EPICENTRE®Madison, Wis.); Paraffin Block RNA Isolation Kit (Ambion, Inc.) and RNeasy™ Mini kit (Qiagen, Chatsworth, Calif.).

The use of FFPE tissues as a source of RNA for RT-PCR has been described previously (Stanta et al., Biotechniques 11:304-308 (1991); Stanta et al., Methods Mol. Biol. 86:23-26 (1998); Jackson et al., Lancet 1:1391 (1989); Jackson et al., J. Clin. Pathol. 43:499-504 (1999); Finke et al., Biotechniques 14:448-453 (1993); Goldsworthy et al., Mol. Carcinog. 25:86-91 (1999); Stanta and Bonin, Biotechniques 24:271-276 (1998); Godfrey et al., J. Mol. Diagnostics 2:84 (2000); Specht et al., J. Mol. Med. 78:B27 (2000); Specht et al., Am. J. Pathol. 158:419-429 (2001)). For quick analysis of the RNA quality, RT-PCR can be performed utilizing a pair of primers targeting a short fragment in a highly expressed gene, for example, actin, ubiquitin, gapdh or other well-described commonly used housekeeping gene. If the cDNA synthesized from the RNA sample can be amplified using this pair of primers, then the sample is suitable for the a quantitative measurements of RNA target sequences by any method preferred, for example, the DASL assay, which requires only a short cDNA fragment for the annealing of query oligonucleotides.

There are numerous tissue banks and collections including exhaustive samples from all stages of a wide variety of disease states, and in particular, Parkinson's disease. The ability to perform genotyping and/or gene expression analysis, including both qualitative and quantitative analysis on these samples enables the application of this methodology to the methods of the invention. In particular, the ability to establish a correlation of gene expression and a known predictor of disease extent and/or outcome by probing the genetic state of tissue samples for which clinical outcome is already known, allows for the establishment of a correlation between a particular molecular signature and the known predictor, such as a Hoehn and Yahr scale score, to derive a score that allows for a more sensitive prognosis than that based on the known predictor alone. The skilled person will appreciate that by building databases of molecular signatures from biological samples of known outcomes, many such correlations can be established, thus allowing both diagnosis and prognosis of any condition. Thus, such approaches may be used to correlate the levels of the markers of the invention, e.g., the markers in Table 2 and Table 5 to a particular stage of Parkinson's disease.

Tissue samples useful for preparing a model for Parkinson's disease prediction include, for example, paraffin and polymer embedded samples, ethanol embedded samples and/or formalin and formaldehyde embedded tissues, although any suitable sample may be used. In general, nucleic acids isolated from archived samples can be highly degraded and the quality of nucleic preparation can depend on several factors, including the sample shelf life, fixation technique and isolation method. However, using the methodologies taught in U.S. Publ. No. 2004/0259105, which have the significant advantage that short or degraded targets can be used for analysis as long as the sequence is long enough to hybridize with the oligonucleotide probes, highly reproducible results can be obtained that closely mimic results found in fresh samples.

Archived tissue samples, which can be used for all methods of the invention, typically have been obtained from a source and preserved. Preferred methods of preservation include, but are not limited to paraffin embedding, ethanol fixation and formalin, including formaldehyde and other derivatives, fixation as are known in the art. A tissue sample may be temporally “old”, e.g. months or years old, or recently fixed. For example, post-surgical procedures generally include a fixation step on excised tissue for histological analysis. In a preferred embodiment, the tissue sample is a diseased tissue sample, particularly a Parkinson's disease tissue.

Thus, an archived sample can be heterogeneous and encompass more than one cell or tissue type. In embodiments directed to methods of establishing a model for Parkinson's disease progression prediction, the tissue sample is one for which patient history and outcome is known. Generally, the invention methods can be practiced with the signature gene sequence contained in an archived sample or can be practiced with signature gene sequences that have been physically separated from the sample prior to performing a method of the invention.

### E. Detection and/or Measurement of Biomarkers

The present invention contemplates any suitable means, techniques, and/or procedures for detecting and/or measuring the markers (e.g., the metabolite, lipid, protein, or nucleic acid markers) of the invention. The skilled artisan will appreciate that the methodologies employed to measure the markers of the invention will depend at least on the type of marker being detected or measured (e.g., lipid, marker, metabolite marker, mRNA marker or polypeptide marker) and the source of the biological sample (e.g., whole blood versus plasma or serum, or urine or other sample). Certain biological samples may also require certain specialized treatments prior to measuring the markers of the invention, e.g., the preparation of mRNA from a biological sample in the case where mRNA markers are being measured.

### 1. Detection of Lipid Markers and Metabolite Markers

A lipid sample may be extracted from a biological sample using any method known in the art such as chloroform-methanol based methods, isopropanol-hexane methods, the Bligh & Dyer lipid extraction method or a modified version thereof, or any combination thereof. Suitable modifications to the Bligh & Dyer method include treatment of crude lipid extracts with lithium methoxide followed by subsequent liquid-liquid extraction to remove generated free fatty acids, fatty acid methyl esters, cholesterol, and water-soluble components that may hinder the shotgun analysis of sphingolipidomes. Since sphingolipids are inert to the described base-treatment, the global analysis and accurate quantitation to assess low and even very low abundant sphingolipids is possible by using a modified Bligh & Dyer method. Following lipid extraction, it may be beneficial to separate the lipids prior to mass spectrometric analysis. Methods for separating lipids are known in the art. Suitable methods include, but are not limited to, chromatography methods such as solid-phase extraction, high performance liquid chromatography (HPLC), normal-phase HPLC, or reverse-phase HPLC. The resultant lipid extracts are then analyzed by mass spectrometric techniques commonly known in the art.

Detection and measurement of metabolites may be carried out using techniques commonly known in the art. For example, metabolomics analysis is described in Tolstikov V, Nikolayev A, Dong S, Zhao G, Kuo M S. Metabolomics Analysis of Metabolic Effects of Nicotinamide Phosphoribosyltransferase (NAMPT) Inhibition on Human Cancer Cells. PLoS One. 2014; 9:e 114019, the contents of which is hereby incorporated herein by reference. Exemplary separation protocols which can be used in metabolite analysis include GC-MS, LC-MS, GC-TOF-MS, HILIC-LC-MS/MS, and RP-LC-HRMS analyses.

Web based databases having high resolution MS data, for example METLIN (see the website metlin.scripps.edu/index.ph,) The Human Metabolome Database (HMDB) (see the website hmdb.ca/, MASSBANK (see the website massbank.jp/, NIST-MS (see the website chemdata.nist.gov/), IDEOME (see the website mzmatch.sourceforge.net/ideom.php), mzCloud (see the website mzcloud.org/) and other libraries can be used for the elemental composition assignment, spectral data comparisons, and detailed manual interpretation.

### 2. Detection of Nucleic Acid Biomarkers

In certain embodiments, the invention involves the detection of nucleic acid markers, e.g., mRNA encoding the protein markers in Table 2 and Table 5. In some embodiments, the diagnostic/prognostic methods of the present invention generally involve the determination of expression levels of one or more genes in a biological sample. Determination of gene expression levels in the practice of the inventive methods may be performed by any suitable method. For example, determination of gene expression levels may be performed by detecting the expression of mRNA expressed from the genes of interest and/or by detecting the expression of a polypeptide encoded by the genes.

For detecting nucleic acids encoding markers of the invention, any suitable method can be used, including, but not limited to, Southern blot analysis, Northern blot analysis, polymerase chain reaction (PCR) (see, for example, U.S. Pat. Nos. 4,683,195; 4,683,202, and 6,040,166; “PCR Protocols: A Guide to Methods and Applications”, Innis et al. (Eds), 1990, Academic Press: New York), reverse transcriptase PCR (RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86: 5673-5677), in situ hybridization, Taqman-based assays (Holland et al., Proc. Natl. Acad. Sci., 1991, 88: 7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan®, etc.

In other embodiments, gene expression levels of markers of interest may be determined by amplifying complementary DNA (cDNA) or complementary RNA (cRNA) produced from mRNA and analyzing it using a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state mRNA level of a large number of genes simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885).

In one particular embodiment, the invention comprises a method for identification of Parkinson's disease in a biological sample by amplifying and detecting nucleic acids corresponding to one or more of the Parkinson's disease markers in Table 2 and Table 5, including one or more of NAP and EMA. The biological sample may be a bodily fluid, for example, blood, serum, plasma, lymph fluid, ascites, serous fluid, pleural effusion, sputum, cerebrospinal fluid, lacrimal fluid, stool, prostatic fluid or urine.

A nucleic acid used as a template for amplification can be isolated from cells contained in the biological sample, according to standard methodologies. (Sambrook et al., 1989) The nucleic acid may be genomic DNA or fractionated or whole cell RNA. Where RNA is used, it may be desired to convert the RNA to a complementary cDNA. In one embodiment, the RNA is whole cell RNA and is used directly as the template for amplification.

Pairs of primers that selectively hybridize to nucleic acids corresponding to any of the Parkinson's disease marker nucleotide sequences identified herein are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology; Bellus, 1994). Following detection, one may compare the results seen in a given patient with a statistically significant reference group of normal patients and Parkinson's disease patients. In this way, it is possible to correlate the amount of nucleic acid detected with various clinical states.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

A number of template dependent processes are available to amplify the nucleic acid sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

In PCR, two primer sequences are prepared which are complementary to regions on opposite complementary strands of the target nucleic acid sequence. An excess of deoxynucleoside triphosphates are added to a reaction mixture along with a DNA polymerase, e.g., Taq polymerase. If the target nucleic acid sequence is present in a sample, the primers will bind to the target nucleic acid and the polymerase will cause the primers to be extended along the target nucleic acid sequence by adding on nucleotides. By raising and lowering the temperature of the reaction mixture, the extended primers will dissociate from the target nucleic acid to form reaction products, excess primers will bind to the target nucleic acid and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641 filed Dec. 21, 1990. Polymerase chain reaction methodologies are well known in the art.

Another method for amplification is the ligase chain reaction (“LCR”), disclosed in European Application No. 320 308, incorporated herein by reference in its entirely. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. Walker et al. (1992), incorporated herein by reference in its entirety.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences also may be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Still other amplification methods described in GB Application No. 2 202 328, and in PCT Application No. PCT/US89/01025, each of which is incorporated herein by reference in its entirety, may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other contemplated nucleic acid amplification procedures include transcription-based amplification systems (TAS), including nucleic acid sequence based amplification (NASBA) and 3SR. Kwoh et al. (1989); Gingeras et al., PCT Application WO 88/10315, incorporated herein by reference in their entirety. In NASBA, the nucleic acids may be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Davey et al., European Application No. 329 822 (incorporated herein by reference in its entirely) disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase 1), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR™.” Frohman (1990) and Ohara et al. (1989), each herein incorporated by reference in their entirety.

Methods based on ligation of two (or more) oligonucleotides in the presence of nucleic acid having the sequence of the resulting “di-oligonucleotide”, thereby amplifying the di-oligonucleotide, also may be used in the amplification step of the present invention. Wu et al. (1989), incorporated herein by reference in its entirety.

Oligonucleotide probes or primers of the present invention may be of any suitable length, depending on the particular assay format and the particular needs and targeted sequences employed. In a preferred embodiment, the oligonucleotide probes or primers are at least 10 nucleotides in length (preferably, 10, 11, 12, 13, 14, 15, 16, 17, 18, 19, 20, 21, 22, 23, 24, 25, 26, 27, 28, 29, 30, 31, 32 . . . ) and they may be adapted to be especially suited for a chosen nucleic acid amplification system and/or hybridization system used. Longer probes and primers are also within the scope of the present invention as well known in the art. Primers having more than 30, more than 40, more than 50 nucleotides and probes having more than 100, more than 200, more than 300, more than 500 more than 800 and more than 1000 nucleotides in length are also covered by the present invention. Of course, longer primers have the disadvantage of being more expensive and thus, primers having between 12 and 30 nucleotides in length are usually designed and used in the art. As well known in the art, probes ranging from 10 to more than 2000 nucleotides in length can be used in the methods of the present invention. As for the % of identity described above, non-specifically described sizes of probes and primers (e.g., 16, 17, 31, 24, 39, 350, 450, 550, 900, 1240 nucleotides, . . . ) are also within the scope of the present invention. In one embodiment, the oligonucleotide probes or primers of the present invention specifically hybridize with a nucleic acid encoding a protein marker in Table 2 and Table 5, including one or more of NAP and EMA, or its complementary sequence. Preferably, the primers and probes of the invention will be chosen to detect a marker in Table 2 and Table 5 which is associated with Parkinson's disease.

In other embodiments, the detection means can utilize a hybridization technique, e.g., where a specific primer or probe is selected to anneal to a target marker of interest, e.g., a nucleic acid encoding a protein marker in Table 2 and Table 5, and thereafter detection of selective hybridization is made. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1994, in Current Protocols in Molecular Biology, John Wiley & Sons Inc., N.Y.).

To enable hybridization to occur under the assay conditions of the present invention, oligonucleotide primers and probes should comprise an oligonucleotide sequence that has at least 70% (at least 71%, 72%, 73%, 74%), preferably at least 75% (75%, 76%, 77%, 78%, 79%, 80%, 81%, 82%, 83%, 84%, 85%, 86%, 87%, 88%, 89%) and more preferably at least 90% (90%, 91%, 92%, 93%, 94%, 95%, 96%, 97%, 98%, 99%, 100%) identity to a portion of a nucleic acid encoding a marker in Table 2 and Table 5, including one or more of NAP and EMA, or a polynucleotide encoding another marker of the invention. Probes and primers of the present invention are those that hybridize under stringent hybridization conditions and those that hybridize to marker homologs of the invention under at least moderately stringent conditions. In certain embodiments probes and primers of the present invention have complete sequence identity (i.e. 100% sequence identity) to the markers of the invention (for example, a nucleic acid encoding a marker in Table 2 and Table 5, including one or more of NAP and EMA, such as a cDNA or mRNA). It should be understood that other probes and primers could be easily designed and used in the present invention based on the markers of the invention disclosed herein by using methods of computer alignment and sequence analysis known in the art (cf. Molecular Cloning: A Laboratory Manual, Third Edition, edited by Cold Spring Harbor Laboratory, 2000).

### 3. Detection of Polypeptide Markers

The present invention contemplates any suitable method for detecting polypeptide markers of the invention. In certain embodiments, the detection method is an immunodetection method involving an antibody that specifically binds to one or more of the markers of the invention, e.g., the markers in Table 2 and Table 5, including one or more of NAP and EMA. The steps of various useful immunodetection methods have been described in the scientific literature, such as, e.g., Nakamura et al. (1987), which is incorporated herein by reference.

In general, the immunobinding methods include obtaining a sample suspected of containing a marker protein, peptide or antibody, and contacting the sample with an antibody or protein or peptide in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing a specific protein, peptide or a corresponding antibody, and contact the sample with an antibody or encoded protein or peptide, as the case may be, and then detect or quantify the amount of immune complexes formed under the specific conditions.

In terms of marker detection, the biological sample analyzed may be any sample that is suspected of containing a Parkinson's disease-specific marker, such as, the markers in Table 2 and Table 5, including one or more of NAP and EMA. The biological sample may be, for example, a homogenized tissue extract, an isolated cell, a cell membrane preparation, separated or purified forms of any of the above protein-containing compositions, or biological fluids including blood or lymphatic fluid.

The chosen biological sample may be contacted with the protein, peptide, or antibody (e.g., as a detection reagent that binds the protein markers in Table 2 and Table 5, including one or more of NAP and EMA, in a biological sample) under conditions effective and for a period of time sufficient to allow the formation of immune complexes (primary immune complexes). Generally, complex formation is a matter of simply adding the composition to the biological sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The encoded protein, peptide, or corresponding antibody (e.g. that selectively binds to a protein marker in Table 2 and Table 5, including one or more of NAP and EMA) employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the diagnosis of conditions such as Parkinson's disease. Here, a biological or clinical sample suspected of containing either the encoded protein or peptide or corresponding antibody is used.

The present invention, in particular, contemplates the use of ELISAs as a type of immunodetection assay. It is contemplated that the marker proteins or peptides of the invention will find utility as immunogens in ELISA assays in diagnosis and prognostic monitoring of Parkinson's disease. Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like also may be used.

In one exemplary ELISA, antibodies binding to the markers of the invention are immobilized onto a selected surface exhibiting protein affinity, such as a well in a polystyrene microtiter plate. Then, a test composition suspected of containing the Parkinson's disease marker antigen, such as a clinical sample, is added to the wells. After binding and washing to remove non-specifically bound immunecomplexes, the bound antigen may be detected. Detection is generally achieved by the addition of a second antibody specific for the target protein, that is linked to a detectable label. This type of ELISA is a simple “sandwich ELISA.” Detection also may be achieved by the addition of a second antibody, followed by the addition of a third antibody that has binding affinity for the second antibody, with the third antibody being linked to a detectable label.

In another exemplary ELISA, the samples suspected of containing the Parkinson's disease marker antigen are immobilized onto the well surface and then contacted with the anti-marker antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Irrespective of the format employed, ELISAs have certain features in common, such as coating, incubating or binding, washing to remove non-specifically bound species, and detecting the bound immunecomplexes. These are described as follows.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control sample and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

The phrase “under conditions effective to allow immunecomplex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25 to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple. Quantitation is then achieved by measuring the degree of color generation, e.g., using a visible spectra spectrophotometer.

The markers of the invention can also be measured, quantitated, detected, and otherwise analyzed using mass spectrometry methods and instrumentation. Protein mass spectrometry refers to the application of mass spectrometry to the study of proteins. Although not intending to be limiting, two approaches are typically used for characterizing proteins using mass spectrometry. In the first, intact proteins are ionized and then introduced to a mass analyzer. This approach is referred to as “top-down” strategy of protein analysis. The two primary methods for ionization of whole proteins are electrospray ionization (ESI) and matrix-assisted laser desorption/ionization (MALDI). In the second approach, proteins are enzymatically digested into smaller peptides using a protease such as trypsin. Subsequently these peptides are introduced into the mass spectrometer and identified by peptide mass fingerprinting or tandem mass spectrometry. Hence, this latter approach (also called “bottom-up” proteomics) uses identification at the peptide level to infer the existence of proteins.

Whole protein mass analysis of the markers of the invention can be conducted using time-of-flight (TOF) MS, or Fourier transform ion cyclotron resonance (FT-ICR). These two types of instruments are useful because of their wide mass range, and in the case of FT-ICR, its high mass accuracy. The most widely used instruments for peptide mass analysis are the MALDI time-of-flight instruments as they permit the acquisition of peptide mass fingerprints (PMFs) at high pace (1 PMF can be analyzed in approx. 10 sec). Multiple stage quadrupole-time-of-flight and the quadrupole ion trap also find use in this application.

The markers of the invention can also be measured in complex mixtures of proteins and molecules that co-exist in a biological medium or sample, however, fractionation of the sample may be required and is contemplated herein. It will be appreciated that ionization of complex mixtures of proteins can result in situation where the more abundant proteins have a tendency to “drown” or suppress signals from less abundant proteins in the same sample. In addition, the mass spectrum from a complex mixture can be difficult to interpret because of the overwhelming number of mixture components. Fractionation can be used to first separate any complex mixture of proteins prior to mass spectrometry analysis. Two methods are widely used to fractionate proteins, or their peptide products from an enzymatic digestion. The first method fractionates whole proteins and is called two-dimensional gel electrophoresis. The second method, high performance liquid chromatography (LC or HPLC) is used to fractionate peptides after enzymatic digestion. In some situations, it may be desirable to combine both of these techniques. Any other suitable methods known in the art for fractionating protein mixtures are also contemplated herein.

Gel spots identified on a 2D Gel are usually attributable to one protein. If the identity of the protein is desired, usually the method of in-gel digestion is applied, where the protein spot of interest is excised, and digested proteolytically. The peptide masses resulting from the digestion can be determined by mass spectrometry using peptide mass fingerprinting. If this information does not allow unequivocal identification of the protein, its peptides can be subject to tandem mass spectrometry for de novo sequencing.

Characterization of protein mixtures using HPLC/MS may also be referred to in the art as “shotgun proteomics” and MuDPIT (Multi-Dimensional Protein Identification Technology). A peptide mixture that results from digestion of a protein mixture is fractionated by one or two steps of liquid chromatography (LC). The eluent from the chromatography stage can be either directly introduced to the mass spectrometer through electrospray ionization, or laid down on a series of small spots for later mass analysis using MALDI.

The polypeptide markers of the present invention (e.g., the markers in Tables 3) can be identified using MS using a variety of techniques, all of which are contemplated herein. Peptide mass fingerprinting uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein was present in the original sample. It will be further appreciated that the development of methods and instrumentation for automated, data-dependent electrospray ionization (ESI) tandem mass spectrometry (MS/MS) in conjunction with microcapillary liquid chromatography (LC) and database searching has significantly increased the sensitivity and speed of the identification of gel-separated proteins. Microcapillary LC-MS/MS has been used successfully for the large-scale identification of individual proteins directly from mixtures without gel electrophoretic separation (Link et al., 1999; Opitek et al., 1997).

Several recent methods allow for the quantitation of proteins by mass spectrometry. For example, stable (e.g., non-radioactive) heavier isotopes of carbon (¹³C) or nitrogen (¹⁵N) can be incorporated into one sample while the other one can be labeled with corresponding light isotopes (e.g. ¹²C and ¹⁴N). The two samples are mixed before the analysis. Peptides derived from the different samples can be distinguished due to their mass difference. The ratio of their peak intensities corresponds to the relative abundance ratio of the peptides (and proteins). The most popular methods for isotope labeling are SILAC (stable isotope labeling by amino acids in cell culture), trypsin-catalyzed ¹⁸O labeling, ICAT (isotope coded affinity tagging), iTRAQ (isobaric tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument. Other types of “label-free” quantitative mass spectrometry, uses the spectral counts (or peptide counts) of digested proteins as a means for determining relative protein amounts.

In one embodiment, any one or more of the polypeptide markers of the invention (e.g., the markers in Table 2 and Table 5, including one or more of NAP and EMA) can be identified and quantified from a complex biological sample using mass spectroscopy in accordance with the following exemplary method, which is not intended to limit the invention or the use of other mass spectrometry-based methods.

In the first step of this embodiment, (A) a biological sample, e.g., a biological sample suspected of having Parkinson's disease, which comprises a complex mixture of protein (including at least one marker of interest) is fragmented and labeled with a stable isotope X. (B) Next, a known amount of an internal standard is added to the biological sample, wherein the internal standard is prepared by fragmenting a standard protein that is identical to the at least one target marker of interest, and labeled with a stable isotope Y. (C) This sample obtained is then introduced in an LC-MS/MS device, and multiple reaction monitoring (MRM) analysis is performed using MRM transitions selected for the internal standard to obtain an MRM chromatogram. (D) The MRM chromatogram is then viewed to identify a target peptide marker derived from the biological sample that shows the same retention time as a peptide derived from the internal standard (an internal standard peptide), and quantifying the target protein marker in the test sample by comparing the peak area of the internal standard peptide with the peak area of the target peptide marker.

Any suitable biological sample may be used as a starting point for LC-MS/MS/MRM analysis, including biological samples derived blood, urine, saliva, hair, cells, cell tissues, and treated products thereof; and protein-containing samples prepared by gene recombination techniques.

Each of the above steps (A) to (D) is described further below.

Step (A) (Fragmentation and Labeling). In step (A), the target protein marker is fragmented to a collection of peptides, which is subsequently labeled with a stable isotope X. To fragment the target protein, for example, methods of digesting the target protein with a proteolytic enzyme (protease) such as trypsin, and chemical cleavage methods, such as a method using cyanogen bromide, can be used. Digestion by protease is preferable. It is known that a given mole quantity of protein produces the same mole quantity for each tryptic peptide cleavage product if the proteolytic digest is allowed to proceed to completion. Thus, determining the mole quantity of tryptic peptide to a given protein allows determination of the mole quantity of the original protein in the sample. Absolute quantification of the target protein can be accomplished by determining the absolute amount of the target protein-derived peptides contained in the protease digestion (collection of peptides). Accordingly, in order to allow the proteolytic digest to proceed to completion, reduction and alkylation treatments are preferably performed before protease digestion with trypsin to reduce and alkylate the disulfide bonds contained in the target protein.

Subsequently, the obtained digest (collection of peptides, comprising peptides of the target marker in the biological sample) is subjected to labeling with a stable isotope X. Examples of stable isotopes X include ¹H and ²H for hydrogen atoms, ¹²C and ¹³C for carbon atoms, and ¹⁴N and ¹⁵N for nitrogen atoms. Any isotope can be suitably selected therefrom. Labeling by a stable isotope X can be performed by reacting the digest (collection of peptides) with a reagent containing the stable isotope. Preferable examples of such reagents that are commercially available include mTRAQ (registered trademark) (produced by Applied Biosystems), which is an amine-specific stable isotope reagent kit. mTRAQ is composed of 2 or 3 types of reagents (mTRAQ-light and mTRAQ-heavy; or mTRAQ-DO, mTRAQ-D4, and mTRAQ-D8) that have a constant mass difference therebetween as a result of isotope-labeling, and that are bound to the N-terminus of a peptide or the primary amine of a lysine residue.

Step (B) (Addition of the Internal Standard). In step (B), a known amount of an internal standard is added to the sample obtained in step (A). The internal standard used herein is a digest (collection of peptides) obtained by fragmenting a protein (standard protein) consisting of the same amino acid sequence as the target protein (target marker) to be measured, and labeling the obtained digest (collection of peptides) with a stable isotope Y. The fragmentation treatment can be performed in the same manner as above for the target protein. Labeling with a stable isotope Y can also be performed in the same manner as above for the target protein. However, the stable isotope Y used herein must be an isotope that has a mass different from that of the stable isotope X used for labeling the target protein digest. For example, in the case of using the aforementioned mTRAQ (registered trademark) (produced by Applied Biosystems), when mTRAQ-light is used to label a target protein digest, mTRAQ-heavy should be used to label a standard protein digest.

Step (C) (LC-MS/MS and MRM Analysis). In step (C), the sample obtained in step (B) is first placed in an LC-MS/MS device, and then multiple reaction monitoring (MRM) analysis is performed using MRM transitions selected for the internal standard. By LC (liquid chromatography) using the LC-MS/MS device, the sample (collection of peptides labeled with a stable isotope) obtained in step (B) is separated first by one-dimensional or multi-dimensional high-performance liquid chromatography. Specific examples of such liquid chromatography include cation exchange chromatography, in which separation is conducted by utilizing electric charge difference between peptides; and reversed-phase chromatography, in which separation is conducted by utilizing hydrophobicity difference between peptides. Both of these methods may be used in combination.

Subsequently, each of the separated peptides is subjected to tandem mass spectrometry by using a tandem mass spectrometer (MS/MS spectrometer) comprising two mass spectrometers connected in series. The use of such a mass spectrometer enables the detection of several fmol levels of a target protein. Furthermore, MS/MS analysis enables the analysis of internal sequence information on peptides, thus enabling identification without false positives. Other types of MS analyzers may also be used, including magnetic sector mass spectrometers (Sector MS), quadrupole mass spectrometers (QMS), time-of-flight mass spectrometers (TOFMS), and Fourier transform ion cyclotron resonance mass spectrometers (FT-ICRMS), and combinations of these analyzers.

Subsequently, the obtained data are put through a search engine to perform a spectral assignment and to list the peptides experimentally detected for each protein. The detected peptides are preferably grouped for each protein, and preferably at least three fragments having an m/z value larger than that of the precursor ion and at least three fragments with an m/z value of, preferably, 500 or more are selected from each MS/MS spectrum in descending order of signal strength on the spectrum. From these, two or more fragments are selected in descending order of strength, and the average of the strength is defined as the expected sensitivity of the MRR transitions. When a plurality of peptides is detected from one protein, at least two peptides with the highest sensitivity are selected as standard peptides using the expected sensitivity as an index.

Step (D) (Quantification of the Target Protein in the Test Sample). Step (D) comprises identifying, in the MRM chromatogram detected in step (C), a peptide derived from the target protein (a target marker of interest) that shows the same retention time as a peptide derived from the internal standard (an internal standard peptide), and quantifying the target protein in the test sample by comparing the peak area of the internal standard peptide with the peak area of the target peptide. The target protein can be quantified by utilizing a calibration curve of the standard protein prepared beforehand.

The calibration curve can be prepared by the following method. First, a recombinant protein consisting of an amino acid sequence that is identical to that of the target marker protein is digested with a protease such as trypsin, as described above. Subsequently, precursor-fragment transition selection standards (PFTS) of a known concentration are individually labeled with two different types of stable isotopes (i.e., one is labeled with a stable isomer used to label an internal standard peptide (labeled with IS), whereas the other is labeled with a stable isomer used to label a target peptide (labeled with T). A plurality of samples are produced by blending a certain amount of the IS-labeled PTFS with various concentrations of the T-labeled PTFS. These samples are placed in the aforementioned LC-MS/MS device to perform MRM analysis. The area ratio of the T-labeled FTFS to the IS-labeled FTFS (T-labeled FTFS/IS-labeled FTFS) on the obtained MRM chromatogram is plotted against the amount of the T-labeled FTFS to prepare a calibration curve. The absolute amount of the target protein contained in the test sample can be calculated by reference to the calibration curve.

### 4. Antibodies and Labels (e.g., Fluorescent Moieties, Dyes)

In some embodiments, the invention provides methods and compositions that include labels for the highly sensitive detection and quantitation of the biomolecules of the invention, e.g., the markers in Table 2 and Table 5, including one or more of NAP and EMA. One skilled in the art will recognize that many strategies can be used for labeling target molecules to enable their detection or discrimination in a mixture of particles (e.g., labeled antibodies to the markers in Table 2 and Table 5, including one or more of NAP and EMA, or labeled secondary antibody, or labeled oligonucleotide probe that specifically hybridizes to mRNA encoding the polypeptide markers in Table 2 and Table 5, including one or more of NAP and EMA). The labels may be attached by any known means, including methods that utilize non-specific or specific interactions of label and target. Labels may provide a detectable signal or affect the mobility of the particle in an electric field. In addition, labeling can be accomplished directly or through binding partners.

In some embodiments, the label comprises a binding partner that binds to the marker of interest, where the binding partner is attached to a fluorescent moiety. The compositions and methods of the invention may utilize highly fluorescent moieties, e.g., a moiety capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. Moieties suitable for the compositions and methods of the invention are described in more detail below.

In some embodiments, the invention provides a label for detecting a biological molecule comprising a binding partner for the biological molecule that is attached to a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the moiety comprises a plurality of fluorescent entities, e.g., about 2 to 4, 2 to 5, 2 to 6, 2 to 7, 2 to 8, 2 to 9, 2 to 10, or about 3 to 5, 3 to 6, 3 to 7, 3 to 8, 3 to 9, or 3 to 10 fluorescent entities. In some embodiments, the moiety comprises about 2 to 4 fluorescent entities. In some embodiments, the biological molecule is a protein or a small molecule. In some embodiments, the biological molecule is a protein. The fluorescent entities can be fluorescent dye molecules. In some embodiments, the fluorescent dye molecules comprise at least one substituted indolium ring system in which the substituent on the 3-carbon of the indolium ring contains a chemically reactive group or a conjugated substance. In some embodiments, the dye molecules are Alexa Fluor molecules selected from the group consisting of Alexa Fluor 488, Alexa Fluor 532, Alexa Fluor 647, Alexa Fluor 680 or Alexa Fluor 700. In some embodiments, the dye molecules are Alexa Fluor molecules selected from the group consisting of Alexa Fluor 488, Alexa Fluor 532, Alexa Fluor 680 or Alexa Fluor 700. In some embodiments, the dye molecules are Alexa Fluor 647 dye molecules. In some embodiments, the dye molecules comprise a first type and a second type of dye molecules, e.g., two different Alexa Fluor molecules, e.g., where the first type and second type of dye molecules have different emission spectra. The ratio of the number of first type to second type of dye molecule can be, e.g., 4 to 1, 3 to 1, 2 to 1, 1 to 1, 1 to 2, 1 to 3 or 1 to 4. The binding partner can be, e.g., an antibody.

In some embodiments, the invention provides a label for the detection of a biological marker of the invention, wherein the label comprises a binding partner for the marker and a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the fluorescent moiety comprises a fluorescent molecule. In some embodiments, the fluorescent moiety comprises a plurality of fluorescent molecules, e.g., about 2 to 10, 2 to 8, 2 to 6, 2 to 4, 3 to 10, 3 to 8, or 3 to 6 fluorescent molecules. In some embodiments, the label comprises about 2 to 4 fluorescent molecules. In some embodiments, the fluorescent dye molecules comprise at least one substituted indolium ring system in which the substituent on the 3-carbon of the indolium ring contains a chemically reactive group or a conjugated substance. In some embodiments, the fluorescent molecules are selected from the group consisting of Alexa Fluor 488, Alexa Fluor 532, Alexa Fluor 647, Alexa Fluor 680 or Alexa Fluor 700. In some embodiments, the fluorescent molecules are selected from the group consisting of Alexa Fluor 488, Alexa Fluor 532, Alexa Fluor 680 or Alexa Fluor 700. In some embodiments, the fluorescent molecules are Alexa Fluor 647 molecules. In some embodiments, the binding partner comprises an antibody. In some embodiments, the antibody is a monoclonal antibody. In other embodiments, the antibody is a polyclonal antibody.

In various embodiments, the binding partner for detecting a marker of interest, e.g., the markers in Table 2 and Table 5, including one or more of NAP and EMA, is an antibody or antigen-binding fragment thereof. The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. An “antigen-binding fragment” of an antibody refers to the part of the antibody that participates in antigen binding. The antigen binding site is formed by amino acid residues of the N-terminal variable (“V”) regions of the heavy (“H”) and light (“L”) chains. It will be appreciated that the choice of epitope or region of the molecule to which the antibody is raised will determine its specificity, e.g., for various forms of the molecule, if present, or for total (e.g., all, or substantially all of the molecule).

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn.; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.).

In some embodiments, the antibody is a polyclonal antibody. In other embodiments, the antibody is a monoclonal antibody.

Antibodies may be prepared by any of a variety of techniques known to those of ordinary skill in the art (see, for example, Harlow and Lane, Antibodies: A Laboratory Manual, Cold Spring Harbor Laboratory, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies.

Monoclonal antibodies may be prepared using hybridoma methods, such as the technique of Kohler and Milstein (Eur. J. Immunol. 6:511-519, 1976), and improvements thereto. These methods involve the preparation of immortal cell lines capable of producing antibodies having the desired specificity. Monoclonal antibodies may also be made by recombinant DNA methods, such as those described in U.S. Pat. No. 4,816,567. DNA encoding antibodies employed in the disclosed methods may be isolated and sequenced using conventional procedures. Recombinant antibodies, antibody fragments, and/or fusions thereof, can be expressed in vitro or in prokaryotic cells (e.g. bacteria) or eukaryotic cells (e.g. yeast, insect or mammalian cells) and further purified as necessary using well known methods.

More particularly, monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells. The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals. Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

The selected hybridomas would then be serially diluted and cloned into individual antibody-producing cell lines, which clones may then be propagated indefinitely to provide MAbs. The cell lines may be exploited for MAb production in two basic ways. A sample of the hybridoma may be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, may then be tapped to provide MAbs in high concentration. The individual cell lines also may be cultured in vitro, where the MAbs are naturally secreted into the culture medium from which they may be readily obtained in high concentrations. MAbs produced by either means may be further purified, if desired, using filtration, centrifugation and various chromatographic methods such as HPLC or affinity chromatography.

Large amounts of the monoclonal antibodies of the present invention also may be obtained by multiplying hybridoma cells in vivo. Cell clones are injected into mammals which are histocompatible with the parent cells, e.g., syngeneic mice, to cause growth of antibody-producing tumors. Optionally, the animals are primed with a hydrocarbon, especially oils such as pristane (tetramethylpentadecane) prior to injection.

In accordance with the present invention, fragments of the monoclonal antibody of the invention may be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention may be synthesized using an automated peptide synthesizer.

Antibodies may also be derived from a recombinant antibody library that is based on amino acid sequences that have been designed in silico and encoded by polynucleotides that are synthetically generated. Methods for designing and obtaining in silico-created sequences are known in the art (Knappik et al., J. Mol. Biol. 296:254:57-86, 2000; Krebs et al., J. Immunol. Methods 254:67-84, 2001; U.S. Pat. No. 6,300,064).

Digestion of antibodies to produce antigen-binding fragments thereof can be performed using techniques well known in the art. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent V_{H}::V_{L} heterodimer including an antigen-binding site which retains much of the antigen recognition and binding capabilities of the native antibody molecule (Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980)).

Antibody fragments that specifically bind to the polypeptide markers disclosed herein can also be isolated from a library of scFvs using known techniques, such as those described in U.S. Pat. No. 5,885,793.

A wide variety of expression systems are available in the art for the production of antibody fragments, including Fab fragments, scFv, VL and VHs. For example, expression systems of both prokaryotic and eukaryotic origin may be used for the large-scale production of antibody fragments. Particularly advantageous are expression systems that permit the secretion of large amounts of antibody fragments into the culture medium. Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins.

Antibodies that bind to the polypeptide markers employed in the present methods are well known to those of skill in the art and in some cases are available commercially or can be obtained without undue experimentation.

In still other embodiments, particularly where oligonucleotides are used as binding partners to detect and hybridize to mRNA markers or other nucleic acid based markers, the binding partners (e.g., oligonucleotides) can comprise a label, e.g., a fluorescent moiety or dye. In addition, any binding partner of the invention, e.g., an antibody, can also be labeled with a fluorescent moiety. The fluorescence of the moiety will be sufficient to allow detection in a single molecule detector, such as the single molecule detectors described herein. A “fluorescent moiety,” as that term is used herein, includes one or more fluorescent entities whose total fluorescence is such that the moiety may be detected in the single molecule detectors described herein. Thus, a fluorescent moiety may comprise a single entity (e.g., a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity may be attached to the binding partner separately or the entities may be attached together, as long as the entities as a group provide sufficient fluorescence to be detected.

Typically, the fluorescence of the moiety involves a combination of quantum efficiency and lack of photobleaching sufficient that the moiety is detectable above background levels in a single molecule detector, with the consistency necessary for the desired limit of detection, accuracy, and precision of the assay. For example, in some embodiments, the fluorescence of the fluorescent moiety is such that it allows detection and/or quantitation of a molecule, e.g., a marker, at a limit of detection of less than about 10, 5, 4, 3, 2, 1, 0.1, 0.01, 0.001, 0.00001, or 0.000001 pg/ml and with a coefficient of variation of less than about 20, 15, 14, 13, 12, 11, 10, 9, 8, 7, 6, 5, 4, 3, 2, 1% or less, e.g., about 10% or less, in the instruments described herein. In some embodiments, the fluorescence of the fluorescent moiety is such that it allows detection and/or quantitation of a molecule, e.g., a marker, at a limit of detection of less than about 5, 1, 0.5, 0.1, 0.05, 0.01, 0.005, 0.001 pg/ml and with a coefficient of variation of less than about 10%, in the instruments described herein. “Limit of detection,” or LoD, as those terms are used herein, includes the lowest concentration at which one can identify a sample as containing a molecule of the substance of interest, e.g., the first non-zero value. It can be defined by the variability of zeros and the slope of the standard curve. For example, the limit of detection of an assay may be determined by running a standard curve, determining the standard curve zero value, and adding 2 standard deviations to that value. A concentration of the substance of interest that produces a signal equal to this value is the “lower limit of detection” concentration.

Furthermore, the moiety has properties that are consistent with its use in the assay of choice. In some embodiments, the assay is an immunoassay, where the fluorescent moiety is attached to an antibody; the moiety must have properties such that it does not aggregate with other antibodies or proteins, or experiences no more aggregation than is consistent with the required accuracy and precision of the assay. In some embodiments, fluorescent moieties that are preferred are fluorescent moieties, e.g., dye molecules that have a combination of 1) high absorption coefficient; 2) high quantum yield; 3) high photostability (low photobleaching); and 4) compatibility with labeling the molecule of interest (e.g., protein) so that it may be analyzed using the analyzers and systems of the invention (e.g., does not cause precipitation of the protein of interest, or precipitation of a protein to which the moiety has been attached).

Any suitable fluorescent moiety may be used. Examples include, but are not limited to, Alexa Fluor dyes (Molecular Probes, Eugene, Oreg.). The Alexa Fluor dyes are disclosed in U.S. Pat. Nos. 6,977,305; 6,974,874; 6,130,101; and 6,974,305 which are herein incorporated by reference in their entirety. Some embodiments of the invention utilize a dye chosen from the group consisting of Alexa Fluor 647, Alexa Fluor 488, Alexa Fluor 532, Alexa Fluor 555, Alexa Fluor 610, Alexa Fluor 680, Alexa Fluor 700, and Alexa Fluor 750. Some embodiments of the invention utilize a dye chosen from the group consisting of Alexa Fluor 488, Alexa Fluor 532, Alexa Fluor 647, Alexa Fluor 700 and Alexa Fluor 750. Some embodiments of the invention utilize a dye chosen from the group consisting of Alexa Fluor 488, Alexa Fluor 532, Alexa Fluor 555, Alexa Fluor 610, Alexa Fluor 680, Alexa Fluor 700, and Alexa Fluor 750. Some embodiments of the invention utilize the Alexa Fluor 647 molecule, which has an absorption maximum between about 650 and 660 nm and an emission maximum between about 660 and 670 nm. The Alexa Fluor 647 dye is used alone or in combination with other Alexa Fluor dyes.

In some embodiments, the fluorescent label moiety that is used to detect a marker in a sample using the analyzer systems of the invention is a quantum dot. Quantum dots (QDs), also known as semiconductor nanocrystals or artificial atoms, are semiconductor crystals that contain anywhere between 100 to 1,000 electrons and range from 2-10 nm. Some QDs can be between 10-20 nm in diameter. QDs have high quantum yields, which makes them particularly useful for optical applications. QDs are fluorophores that fluoresce by forming excitons, which are similar to the excited state of traditional fluorophores, but have much longer lifetimes of up to 200 nanoseconds. This property provides QDs with low photobleaching. The energy level of QDs can be controlled by changing the size and shape of the QD, and the depth of the QDs' potential. One optical feature of small excitonic QDs is coloration, which is determined by the size of the dot. The larger the dot, the redder, or more towards the red end of the spectrum the fluorescence. The smaller the dot, the bluer or more towards the blue end it is. The bandgap energy that determines the energy and hence the color of the fluoresced light is inversely proportional to the square of the size of the QD. Larger QDs have more energy levels which are more closely spaced, thus allowing the QD to absorb photons containing less energy, i.e., those closer to the red end of the spectrum. Because the emission frequency of a dot is dependent on the bandgap, it is possible to control the output wavelength of a dot with extreme precision. In some embodiments the protein that is detected with the single molecule analyzer system is labeled with a QD. In some embodiments, the single molecule analyzer is used to detect a protein labeled with one QD and using a filter to allow for the detection of different proteins at different wavelengths.

### F. Isolated Biomarkers

### 1. Isolated Polypeptide Biomarkers

One aspect of the invention pertains to isolated marker proteins and biologically active portions thereof, as well as polypeptide fragments suitable for use as immunogens to raise antibodies directed against a marker protein or a fragment thereof. In one embodiment, the native marker protein can be isolated from cells or tissue sources by an appropriate purification scheme using standard protein purification techniques. In another embodiment, a protein or peptide comprising the whole or a segment of the marker protein is produced by recombinant DNA techniques. Alternative to recombinant expression, such protein or peptide can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a marker protein include polypeptides comprising amino acid sequences sufficiently identical to or derived from the amino acid sequence of the marker protein, which include fewer amino acids than the full length protein, and exhibit at least one activity of the corresponding full-length protein. Typically, biologically active portions comprise a domain or motif with at least one activity of the corresponding full-length protein. A biologically active portion of a marker protein of the invention can be a polypeptide which is, for example, 10, 25, 50, 100 or more amino acids in length. Moreover, other biologically active portions, in which other regions of the marker protein are deleted, can be prepared by recombinant techniques and evaluated for one or more of the functional activities of the native form of the marker protein.

Preferred marker proteins are encoded by nucleotide sequences provided in the sequence listing. Other useful proteins are substantially identical (e.g., at least about 40%, preferably 50%, 60%, 70%, 80%, 90%, 91%, 92%, 93%, 94%, 95%, 96%, 97%, 98% or 99%) to one of these sequences and retain the functional activity of the corresponding naturally-occurring marker protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. Preferably, the percent identity between the two sequences is calculated using a global alignment. Alternatively, the percent identity between the two sequences is calculated using a local alignment. The percent identity between the two sequences is a function of the number of identical positions shared by the sequences (i.e., % identity=# of identical positions/total # of positions (e.g., overlapping positions)×100). In one embodiment the two sequences are the same length. In another embodiment, the two sequences are not the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTX programs of Altschul, et al. (1990) J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTP program, score=50, wordlength=3 to obtain amino acid sequences homologous to a protein molecules of the invention. To obtain gapped alignments for comparison purposes, a newer version of the BLAST algorithm called Gapped BLAST can be utilized as described in Altschul et al. (1997) Nucleic Acids Res. 25:3389-3402, which is able to perform gapped local alignments for the programs BLASTN, BLASTP and BLASTX. Alternatively, PSI-Blast can be used to perform an iterated search which detects distant relationships between molecules. When utilizing BLAST, Gapped BLAST, and PSI-Blast programs, the default parameters of the respective programs (e.g., BLASTX and BLASTN) can be used. See the NCBI website. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

Another aspect of the invention pertains to antibodies directed against a protein of the invention. In preferred embodiments, the antibodies specifically bind a marker protein or a fragment thereof. The terms “antibody” and “antibodies” as used interchangeably herein refer to immunoglobulin molecules as well as fragments and derivatives thereof that comprise an immunologically active portion of an immunoglobulin molecule, (i.e., such a portion contains an antigen binding site which specifically binds an antigen, such as a marker protein, e.g., an epitope of a marker protein). An antibody which specifically binds to a protein of the invention is an antibody which binds the protein, but does not substantially bind other molecules in a sample, e.g., a biological sample, which naturally contains the protein. Examples of an immunologically active portion of an immunoglobulin molecule include, but are not limited to, single-chain antibodies (scAb), F(ab) and F(ab′)₂ fragments.

An isolated protein of the invention or a fragment thereof can be used as an immunogen to generate antibodies. The full-length protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the proteins of the invention, and encompasses at least one epitope of the protein such that an antibody raised against the peptide forms a specific immune complex with the protein. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions. In preferred embodiments, an isolated marker protein or fragment thereof is used as an immunogen.

The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope. Preferred polyclonal and monoclonal antibody compositions are ones that have been selected for antibodies directed against a protein of the invention. Particularly preferred polyclonal and monoclonal antibody preparations are ones that contain only antibodies directed against a marker protein or fragment thereof. Methods of making polyclonal, monoclonal, and recombinant antibody and antibody fragments are well known in the art.

### 2. Isolated Nucleic Acid Biomarkers

One aspect of the invention pertains to isolated nucleic acid molecules, including nucleic acids which encode a marker protein or a portion thereof. Isolated nucleic acids of the invention also include nucleic acid molecules sufficient for use as hybridization probes to identify marker nucleic acid molecules, and fragments of marker nucleic acid molecules, e.g., those suitable for use as PCR primers for the amplification of a specific product or mutation of marker nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. In one embodiment, an “isolated” nucleic acid molecule (preferably a protein-encoding sequences) is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. In another embodiment, an “isolated” nucleic acid molecule, such as a cDNA molecule, can be substantially free of other cellular material, or culture medium when produced by recombinant techniques, or substantially free of chemical precursors or other chemicals when chemically synthesized. A nucleic acid molecule that is substantially free of cellular material includes preparations having less than about 30%, 20%, 10%, or 5% of heterologous nucleic acid (also referred to herein as a “contaminating nucleic acid”).

A nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, NY, 1989).

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, nucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a marker nucleic acid or to the nucleotide sequence of a nucleic acid encoding a marker protein. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker nucleic acid or which encodes a marker protein. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 15, more preferably at least about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. In certain embodiments, the probes hybridize to nucleic acid sequences that traverse splice junctions. The probe comprises a label group attached thereto, e.g., a radioisotope, a fluorescent compound, an enzyme, or an enzyme co-factor. Such probes can be used as part of a diagnostic test kit or panel for identifying cells or tissues which express or mis-express the protein, such as by measuring levels of a nucleic acid molecule encoding the protein in a sample of cells from a subject, e.g., detecting mRNA levels or determining whether a gene encoding the protein or its translational control sequences have been mutated or deleted.

The invention further encompasses nucleic acid molecules that differ, due to degeneracy of the genetic code, from the nucleotide sequence of nucleic acids encoding a marker protein (e.g., protein having the sequence provided in the sequence listing), and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation and changes known to occur in cancer. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a marker nucleic acid or to a nucleic acid encoding a marker protein. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

### G. Biomarker Applications

The invention provides methods for diagnosing a disease, e.g., Parkinson's disease, in a subject. The invention further provides methods for prognosing or monitoring progression of Parkinson's disease or monitoring response to a therapeutic for Parkinson's disease. In one aspect, the present invention constitutes an application of diagnostic information obtainable by the methods of the invention in connection with analyzing, detecting, and/or measuring the Parkinson's disease markers of the present invention, for example, one or more of NAP and EMA or the markers in Table 2 and Table 5, which goes well beyond the discovered correlation between Parkinson's disease and the markers of the invention.

For example, when executing the methods of the invention for detecting and/or measuring a polypeptide marker of the present invention, as described herein, one contacts a biological sample with a detection reagent, e.g, a monoclonal antibody, which selectively binds to the marker of interest, forming a protein-protein complex, which is then further detected either directly (if the antibody comprises a label) or indirectly (if a secondary detection reagent is used, e.g., a secondary antibody, which in turn is labeled). Thus, the method of the invention transforms the polypeptide markers of the invention to a protein-protein complex that comprises either a detectable primary antibody or a primary and further secondary antibody. Forming such protein-protein complexes is required in order to identify the presence of the polypeptide marker of interest and necessarily changes the physical characteristics and properties of the marker of interest as a result of conducting the methods of the invention.

The same principal applies when conducting the methods of the invention for detecting nucleic acid markers of the invention. In particular, when amplification methods are used to detect a marker of the invention (e.g., an mRNA encoding a polypeptide marker in Table 2 and Table 5, including one or more of NAP and EMA), the amplification process, in fact, results in the formation of a new population of amplicons—i.e., molecules that are newly synthesized and which were not present in the original biological sample, thereby physically transforming the biological sample. Similarly, when hybridization probes are used to detect a target marker, a physical new species of molecules is in effect created by the hybridization of the probes (optionally comprising a label) to the target marker mRNA (or other nucleic acid), which is then detected. Such polynucleotide products are effectively newly created or formed as a consequence of carrying out the method of the invention.

The invention provides, in one embodiment, methods for diagnosing a disease, e.g., Parkinson's disease. The methods of the present invention can be practiced in conjunction with any other method used by the skilled practitioner to prognose the occurrence of Parkinson's disease and/or the survival of a subject being treated for Parkinson's disease. The diagnostic and prognostic methods provided herein can be used to determine if additional and/or more invasive tests or monitoring should be performed on a subject. It is understood that a disease as complex as Parkinson's disease is rarely diagnosed using a single test. Therefore, it is understood that the diagnostic, prognostic, and monitoring methods provided herein are typically used in conjunction with other methods known in the art. For example, the methods of the invention may be performed in conjunction with imaging analysis, and/or physical exam. Cytological methods would include immunohistochemical or immunofluorescence detection (and quantitation if appropriate) of any other molecular marker either by itself, in conjunction with other markers. Other methods would include detection of other markers by in situ PCR, or by extracting tissue and quantitating other markers by real time PCR. PCR is defined as polymerase chain reaction.

Methods for assessing disease progression during a treatment regimen, e.g., levodopa, surgery, or any other therapeutic approach useful for treating Parkinson's disease in a subject are also provided. In these methods the amount of marker in a pair of samples (a first sample obtained from the subject at an earlier time point or prior to the treatment regimen and a second sample obtained from the subject at a later time point, e.g., at a later time point when the subject has undergone at least a portion of the treatment regimen) is assessed. It is understood that the methods of the invention include obtaining and analyzing more than two samples (e.g., 3, 4, 5, 6, 7, 8, 9, or more samples) at regular or irregular intervals for assessment of marker levels. Pairwise comparisons can be made between consecutive or non-consecutive subject samples. Trends of marker levels and rates of change of marker levels can be analyzed for any two or more consecutive or non-consecutive subject samples.

The invention also provides a method for determining the rate of progression of Parkinson's disease. The method comprises determining the amount of a marker present in a sample and comparing the amount to a control amount of the marker present in one or more control samples, thereby determining the rate of progression of Parkinson's disease. Marker levels can be compared to marker levels in samples obtained at different times from the same subject or marker levels from normal or abnormal Parkinson's disease subjects. A rapid increase in the level of marker may be indicative of rapid progression of Parkinson's disease compared to a slow increase or no increase or change in the marker level.

The methods of the invention may also be used to select a compound that is capable of modulating, i.e., decreasing, the progression of Parkinson's disease. In this method, a Parkinson's disease cell is contacted with a test compound, and the ability of the test compound to modulate the expression and/or activity of a marker in the invention in the Parkinson's disease cell is determined, thereby selecting a compound that is capable of modulating aggressiveness of Parkinson's disease.

Using the methods described herein, a variety of molecules, may be screened in order to identify molecules which modulate, e.g., increase or decrease the expression and/or activity of a marker of the invention, e.g., the markers in Table 2 and Table 5, including one or more of NAP and EMA. Compounds so identified can be provided to a subject in order to slow the progression of Parkinson's disease in the subject, or to treat Parkinson's disease in the subject.

The present invention pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for detecting the level of expression of one or more marker proteins or nucleic acids, in order to determine whether an individual is at risk of developing a disease or disorder, such as, for example, Parkinson's disease. Such assays can be used for prognostic or predictive purposes to thereby prophylactically treat an individual prior to the onset of the disorder.

Yet another aspect of the invention pertains to monitoring the influence of agents (e.g., drugs or other therapeutic compounds) on the level of a marker of the invention in clinical trials. These and other applications are described in further detail in the following sections.

### 1. Diagnostic Assays

An exemplary method for detecting the presence or absence or change of expression level of a marker protein or nucleic acid in a biological sample involves obtaining a biological sample (e.g. a Parkinson's disease associated body fluid) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo.

Methods provided herein for detecting the presence, absence, change of expression level of a marker protein or nucleic acid in a biological sample include obtaining a biological sample from a subject that may or may not contain the marker protein or nucleic acid to be detected, contacting the sample with a marker-specific binding agent (i.e., one or more marker-specific binding agents) that is capable of forming a complex with the marker protein or nucleic acid to be detected, and contacting the sample with a detection reagent for detection of the marker-marker-specific binding agent complex, if formed. It is understood that the methods provided herein for detecting an expression level of a marker in a biological sample includes the steps to perform the assay. In certain embodiments of the detection methods, the level of the marker protein or nucleic acid in the sample is none or below the threshold for detection.

The methods include formation of either a transient or stable complex between the marker and the marker-specific binding agent. The methods require that the complex, if formed, be formed for sufficient time to allow a detection reagent to bind the complex and produce a detectable signal (e.g., fluorescent signal, a signal from a product of an enzymatic reaction, e.g., a peroxidase reaction, a phosphatase reaction, a beta-galactosidase reaction, or a polymerase reaction).

In certain embodiments, all markers are detected using the same method. In certain embodiments, all markers are detected using the same biological sample (e.g., same body fluid or tissue). In certain embodiments, different markers are detected using various methods. In certain embodiments, markers are detected in different biological samples.

### 2. Protein Detection

In certain embodiments of the invention, the marker to be detected is a protein. Proteins are detected using a number of assays in which a complex between the marker protein to be detected and the marker specific binding agent would not occur naturally, for example, because one of the components is not a naturally occurring compound or the marker for detection and the marker specific binding agent are not from the same organism (e.g., human marker proteins detected using marker-specific binding antibodies from mouse, rat, or goat). In a preferred embodiment of the invention, the marker protein for detection is a human marker protein. In certain detection assays, the human markers for detection are bound by marker-specific, non-human antibodies, thus, the complex would not be formed in nature. The complex of the marker protein can be detected directly, e.g., by use of a labeled marker-specific antibody that binds directly to the marker, or by binding a further component to the marker-marker-specific antibody complex. In certain embodiments, the further component is a second marker-specific antibody capable of binding the marker at the same time as the first marker-specific antibody. In certain embodiments, the further component is a secondary antibody that binds to a marker-specific antibody, wherein the secondary antibody preferably linked to a detectable label (e.g., fluorescent label, enzymatic label, biotin). When the secondary antibody is linked to an enzymatic detectable label (e.g., a peroxidase, a phosphatase, a beta-galactosidase), the secondary antibody is detected by contacting the enzymatic detectable label with an appropriate substrate to produce a colorimetric, fluorescent, or other detectable, preferably quantitatively detectable, product. Antibodies for use in the methods of the invention can be polyclonal, however, in a preferred embodiment monoclonal antibodies are used. An intact antibody, or a fragment or derivative thereof (e.g., Fab or F(ab′)₂) can be used in the methods of the invention. Such strategies of marker protein detection are used, for example, in ELISA, RIA, western blot, and immunofluorescence assay methods.

In certain detection assays, the marker present in the biological sample for detection is an enzyme and the detection reagent is an enzyme substrate. For example, the enzyme can be a protease and the substrate can be any protein that includes an appropriate protease cleavage site. Alternatively, the enzyme can be a kinase and the substrate can be any substrate for the kinase. In preferred embodiments, the substrate which forms a complex with the marker enzyme to be detected is not the substrate for the enzyme in a human subject.

In certain embodiments, the marker-marker-specific binding agent complex is attached to a solid support for detection of the marker. The complex can be formed on the substrate or formed prior to capture on the substrate. For example, in an ELISA, RIA, immunoprecipitation assay, western blot, immunofluorescence assay, in gel enzymatic assay the marker for detection is attached to a solid support, either directly or indirectly. In an ELISA, RIA, or immunofluorescence assay, the marker is typically attached indirectly to a solid support through an antibody or binding protein. In a western blot or immunofluorescence assay, the marker is typically attached directly to the solid support. For in-gel enzyme assays, the marker is resolved in a gel, typically an acrylamide gel, in which a substrate for the enzyme is integrated.

### 3. Nucleic Acid Detection

In certain embodiments of the invention, the marker is a nucleic acid. Nucleic acids are detected using a number of assays in which a complex between the marker nucleic acid to be detected and a marker-specific probe would not occur naturally, for example, because one of the components is not a naturally occurring compound. In certain embodiments, the analyte comprises a nucleic acid and the probe comprises one or more synthetic single stranded nucleic acid molecules, e.g., a DNA molecule, a DNA-RNA hybrid, a PNA, or a modified nucleic acid molecule containing one or more artificial bases, sugars, or backbone moieties. In certain embodiments, the synthetic nucleic acid is a single stranded is a DNA molecule that includes a fluorescent label. In certain embodiments, the synthetic nucleic acid is a single stranded oligonucleotide molecule of about 12 to about 50 nucleotides in length. In certain embodiments, the nucleic acid to be detected is an mRNA and the complex formed is an mRNA hybridized to a single stranded DNA molecule that is complementary to the mRNA. In certain embodiments, an RNA is detected by generation of a DNA molecule (i.e., a cDNA molecule) first from the RNA template using the single stranded DNA that hybridizes to the RNA as a primer, e.g., a general poly-T primer to transcribe poly-A RNA. The cDNA can then be used as a template for an amplification reaction, e.g., PCR, primer extension assay, using a marker-specific probe. In certain embodiments, a labeled single stranded DNA can be hybridized to the RNA present in the sample for detection of the RNA by fluorescence in situ hybridization (FISH) or for detection of the RNA by northern blot.

For example, in vitro techniques for detection of mRNA include northern hybridizations, in situ hybridizations, and rtPCR. In vitro techniques for detection of genomic DNA include Southern hybridizations. Techniques for detection of mRNA include PCR, northern hybridizations and in situ hybridizations. Methods include both qualitative and quantitative methods.

A general principle of such diagnostic, prognostic, and monitoring assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways known in the art, e.g., ELISA assay, PCR, FISH.

### 4. Detection of Marker Levels

Marker levels can be detected based on the absolute level or a normalized or relative expression level. Detection of absolute marker levels may be preferable when monitoring the treatment of a subject or in determining if there is a change in the Parkinson's disease status of a subject. For example, the level of one or more markers, such as NAP and/or EMA, can be monitored in a subject undergoing treatment for Parkinson's disease, e.g., at regular intervals, such a monthly intervals. A modulation in the level of one or more markers can be monitored over time to observe trends in changes in marker levels. Levels of the markers of the invention, e.g., NAP and/or EMA or the markers in Table 2 and Table 5 in the subject may be higher than the level of those markers in a normal sample, but may be lower than the prior level, thus indicating a benefit of the treatment regimen for the subject. Similarly, rates of change of marker levels can be important in a subject who is not subject to active treatment for Parkinson's disease. Changes, or not, in marker levels may be more relevant to treatment decisions for the subject than marker levels present in the population. Rapid changes in marker levels in a subject may be indicative of a rapid progression in Parkinson's disease, even if the markers are within normal ranges for the population.

As an alternative to making determinations based on the absolute level of the marker, determinations may be based on the normalized expression level of the marker. Marker levels are normalized by correcting the absolute level of a marker by comparing its level to the level of a compound that is not a marker, e.g., by comparing the expression of a protein marker to the expression of a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-Parkinson's disease sample, or between samples from different sources.

Alternatively, the marker level can be provided as a relative marker level as compared to an appropriate control, e.g., population control, adjacent normal tissue control, earlier time point control, etc. Preferably, the samples used in the baseline determination will be from subjects that do not have Parkinson's disease. The choice of the cell source is dependent on the use of the relative marker level. Using marker levels found in normal tissues as a mean marker level score aids in validating whether the marker assayed is Parkinson's disease specific (versus non-diseased samples). In addition, as more data is accumulated, the mean marker level value can be revised, providing improved relative marker level values based on accumulated data. Marker level data from Parkinson's disease samples provides a means for grading the severity of the Parkinson's disease state.

### 5. Diagnostic, Prognostic, and Treatment Methods

The invention provides methods for detecting Parkinson's disease in a subject by
(1) contacting a biological sample from a subject with a panel of one or more detection reagents wherein each detection reagent is specific for one marker of Parkinson's disease; wherein the marker of Parkinson's disease is selected from the markers in Table 2 and Table 5;
(2) measuring the amount of each Parkinson's disease related marker detected in the biological sample by each detection reagent; and
(3) comparing the level of one or more markers of Parkinson's disease in the biological sample obtained from the subject with a level of the one or more markers of Parkinson's disease in a control sample, thereby detecting Parkinson's disease.

The invention also provides methods for monitoring the treatment of Parkinson's disease in a subject by
(1) contacting a first biological sample obtained from the subject prior to administering at least a portion of a treatment regimen to the subject with a panel of one or more detection reagents wherein each detection reagent is specific for one marker of Parkinson's disease; wherein the marker of Parkinson's disease is NAP and/or EMA or, or others selected from the group consisting of the markers in Table 2 and Table 5;
(2) contacting a second biological sample obtained from the subject after administering at least a portion of a treatment regimen to the subject with a panel of one or more detection reagents wherein each detection reagent is specific for one marker of Parkinson's disease; wherein the marker of Parkinson's disease is NAP and/or EMA or, or others selected from the group consisting of the markers in Table 2 and Table 5;
(3) measuring the amount of the marker of Parkinson's disease in the first biological sample and the second biological sample by each detection reagent; and
(4) comparing the level of the marker of Parkinson's disease in the first sample with the level of one or more of markers of Parkinson's disease in the second sample, thereby monitoring the treatment of Parkinson's disease in the subject.

The invention provides methods of selecting for administration of active treatment or against administration of active treatment of Parkinson's disease in a subject by
(1) contacting a first biological sample obtained from the subject prior to administering a treatment regimen to the subject with a panel of one or more detection reagents wherein each detection reagent is specific for one marker of Parkinson's disease; wherein the marker of Parkinson's disease is NAP and/or EMA or, or others selected from the group consisting of the markers in Table 2 and Table 5;
(2) contacting a second biological sample obtained from the subject after administering a treatment regimen to the subject with a panel of one or more detection reagents wherein each detection reagent is specific for one marker of Parkinson's disease; wherein the marker of Parkinson's disease is NAP and/or EMA or, or others selected from the group consisting of the markers in Table 2 and Table 5;
(3) measuring the level of each marker of Parkinson's disease detected in the first biological sample and the second biological sample by each detection reagent; and
(4) comparing the level of one or more markers of Parkinson's disease in the first sample with the level of one or more markers of Parkinson's disease in the second sample, wherein selecting for administration of active treatment or against administration of active treatment of Parkinson's disease is based on the presence or absence of changes in the level of one or more markers between the first sample and the second sample.

In certain embodiments of the diagnostic and monitoring methods provided herein, the one or more markers of Parkinson's disease are two or more markers. In certain embodiments of the diagnostic and monitoring methods provided herein, the one or more markers of Parkinson's disease are three or more markers. In certain embodiments of the diagnostic and monitoring methods provided herein, the one or more markers of Parkinson's disease are four or more markers. In certain embodiments of the diagnostic and monitoring methods provided herein, the one or more markers of Parkinson's disease are five or more markers. In certain embodiments of the diagnostic and monitoring methods provided herein, the one or more markers of Parkinson's disease are six or more markers. In certain embodiments of the diagnostic and monitoring methods provided herein, the one or more markers of Parkinson's disease are seven or more markers. In certain embodiments of the diagnostic and monitoring methods provided herein, the one or more markers of Parkinson's disease are eight or more markers. In certain embodiments of the diagnostic and monitoring methods provided herein, the one or more markers of Parkinson's disease are nine or more markers.

In certain embodiments of the diagnostic methods provided herein, a difference in the level of one or more markers of Parkinson's disease such as NAP and/or EMA, or others selected from the group consisting of the markers in Table 2 and Table 5 in the biological sample as compared to the level of the one or more markers of Parkinson's disease in a normal control sample is an indication that the subject is afflicted with Parkinson's disease. In certain embodiments of the diagnostic methods provided herein, no difference in the detected level of NAP and/or EMA, or that of the other markers in Table 2 and Table 5 in the biological sample as compared to the level in a normal control sample is an indication that the subject is not afflicted with Parkinson's disease or not predisposed to developing Parkinson's disease. In particular embodiments of the diagnostic methods provided herein, the difference in the level of one or more markers of Parkinson's disease is an increase in the level of the one or more markers. In other embodiments of the diagnostic methods provided herein, the difference in the level of one or more markers of Parkinson's disease is a decrease in the level of the one or more markers.

In certain embodiments of the diagnostic methods provided herein, a difference in the level of one or more markers of Parkinson's disease such as NAP and/or EMA, or others selected from the group consisting of the markers in Table 2 and Table 5 in the biological sample as compared to the level of expression of the one or more markers of Parkinson's disease in a normal control sample is an indication that the subject is predisposed to developing Parkinson's disease. In particular embodiments of the diagnostic methods provided herein, the difference in the level of one or more markers of Parkinson's disease is an increase in the level of the one or more markers. In other embodiments of the diagnostic methods provided herein, the difference in the level of one or more markers of Parkinson's disease is a decrease in the level of the one or more markers.

In certain embodiments of the monitoring methods provided herein, no change in the detected level of any of the one or more markers of Parkinson's disease such as NAP and/or EMA or that of others selected from the group consisting of the markers in Table 2 and Table 5 in the second sample as compared to the level of the one or more markers of Parkinson's disease in the first sample is an indication that the therapy is efficacious for treating Parkinson's disease in the subject. In certain embodiments of the monitoring methods provided herein, the methods further comprise comparing the level of NAP and/or EMA or that of one or more markers of Parkinson's disease selected from the group consisting of the markers in Table 2 and Table 5 in the first sample or the level of NAP and/or EMA or that of one or more markers of Parkinson's disease selected from the group consisting of the markers in Table 2 and Table 5 in the second sample with the level of the one or more markers of Parkinson's disease in a control sample.

In certain embodiments of the monitoring methods provided herein, a difference in the level of NAP and/or EMA or that of one or more markers of Parkinson's disease selected from the group consisting of the markers in Table 2 and Table 5 in the second sample as compared to the level of NAP and/or EMA or that of one or more markers of Parkinson's disease in the first sample is an indication for selection of active treatment of Parkinson's disease in the subject. In certain embodiments of the monitoring methods provided herein, no difference in the detected level of NAP and/or EMA or that of any of the one or more markers of Parkinson's disease selected from the group consisting of the markers in Table 2 and Table 5 in the second sample as compared to the level of NAP and/or EMA or that of one or more markers of Parkinson's disease in the first sample is an indication against selection of active treatment of Parkinson's disease in the subject. In certain embodiments of the monitoring methods provided herein, a difference in the level of NAP and/or EMA or that of markers in Table 2 and Table 5 in the second sample as compared to the level in the first sample is an indication that the therapy is not efficacious in the treatment of Parkinson's disease. In particular embodiments of the monitoring methods provided herein, the difference in the level of one or more markers of Parkinson's disease is an increase in the level of the one or more markers. In other embodiments of the monitoring methods provided herein, the difference in the level of one or more markers of Parkinson's disease is a decrease in the level of the one or more markers.

In certain embodiments of the diagnostic and monitoring methods provided herein, the one or more markers of Parkinson's disease is NAP and/or EMA or is selected from the group consisting of the protein markers of Table 2 and Table 5. In certain embodiments of the diagnostic and monitoring methods provided herein, the one or more markers of Parkinson's disease is NAP and/or EMA or is selected from the group consisting of a nucleic acid encoding the protein markers of Table 2 and Table 5.

In certain embodiments of the monitoring methods provided herein, modulation of the level of the NAP and/or EMA or that of one or more markers of Parkinson's disease selected from the group consisting of the markers in Table 2 and Table 5 in the second sample as compared to the level of the corresponding marker(s) in the first sample is indicative of a change in Parkinson's disease status in response to treatment of the Parkinson's disease in the subject.

In any of the aforementioned embodiments, the methods may also include a step of determining whether a subject having Parkinson's disease or who is being treated for Parkinson's disease is responsive to a particular treatment. Such a step can include, for example, measuring the level of NAP and/or EMA or that of one or more markers of Parkinson's disease selected from the group consisting of the markers in Table 2 and Table 5 prior to administering an anti-Parkinson's disease treatment, and measuring the level of expression of NAP and/or EMA or that of one or more markers of Parkinson's disease selected from the group consisting of the markers in Table 2 and Table 5 after administering the anti-Parkinson's disease treatment, and comparing the level of the markers before and after treatment. Determining that the Parkinson's disease is responsive to the treatment if the level of NAP and/or EMA or that of one or more markers is different before treatment as compared to after treatment. The method may further include the step of adjusting the treatment to a higher dose in order to increase the responsiveness to the treatment, or adjusting the treatment to a lower dose in order to decrease the responsiveness to the treatment.

In any of the aforementioned embodiments, the methods may also include a step of determining whether a subject having Parkinson's disease or who is being treated for Parkinson's disease is responsive to a particular treatment. Such a step can include, for example, measuring the level of NAP and/or EMA or that of one or more markers of Parkinson's disease selected from the group consisting of the markers in Table 2 and Table 5 prior to administering an anti-Parkinson's disease treatment, and measuring the level of expression of NAP and/or EMA or that of one or more markers of Parkinson's disease selected from the group consisting of the markers in Table 2 and Table 5 after administering the anti-Parkinson's disease treatment, and comparing the expression level before and after treatment. The method may also comprise determining that the Parkinson's disease is responsive to the treatment if the level of NAP and/or EMA or that of one or more markers is different than before treatment as compared to after treatment. The method may further include the step of adjusting the treatment to a higher dose in order to increase the responsiveness to the treatment, or adjusting the treatment to a lower dose in order to decrease the responsiveness to the treatment.

In any of the aforementioned embodiments, the methods may also include a step of determining whether a subject having Parkinson's disease or who is being treated for Parkinson's disease is not responsive to a particular treatment. Such a step can include, for example, measuring the level of NAP and/or EMA or that of one or more markers of Parkinson's disease selected from the group consisting of the markers in Table 2 and Table 5 prior to administering an anti-Parkinson's disease treatment, and measuring the level of NAP and/or EMA, or that of one or more markers of Parkinson's disease selected from the group consisting of the markers in Table 2 and Table 5 after administering the anti-Parkinson's disease treatment, and comparing the level of the marker before and after treatment. Determining that the Parkinson's disease is not responsive to the treatment if the level of NAP and/or EMA, or that of one or more markers is different after treatment as compared to before treatment. The method may further include the step of adjusting the treatment to a higher dose in order to increase the responsiveness to the treatment.

In certain embodiments the diagnostic and monitoring methods provided herein further comprise comparing the detected level of NAP and/or EMA, or that of one or more markers in the biological samples with one or more control samples wherein the control sample is one or more of a sample from the same subject at an earlier time point than the biological sample.

Certain other embodiments of the diagnostic and monitoring methods further comprise determining the particular stage or grade of Parkinson's disease, e.g., Hoehn-Yahr scale 0, scale 1, scale 1.5, scale 2, scale 2.5, scale 3, scale 4, or scale 5 Parkinson's disease. In other embodiments, the present invention also involves the analysis and consideration of any clinical and/or patient-related health data, for example, data obtained from an Electronic Medical Record (e.g., collection of electronic health information about individual patients or populations relating to various types of data, such as, demographics, medical history, medication and allergies, immunization status, laboratory test results, radiology images, vital signs, personal statistics like age and weight, and billing information).

In certain embodiments the diagnostic and monitoring methods provided herein further comprising obtaining a subject sample.

In certain embodiments the diagnostic and monitoring methods provided herein further comprising selecting a treatment regimen for the subject based on the level of NAP and/or EMA, or that of one or more of the markers of Parkinson's disease selected from the group consisting of the markers in Table 2 and Table 5.

In certain embodiments the diagnostic and monitoring methods provided herein further comprise selecting a subject for having or being suspected of having Parkinson's disease.

In certain embodiments the diagnostic and monitoring methods provided herein further comprising treating the subject with a regimen including one or more treatments selected from the group consisting of surgery and levodopa.

In certain embodiments the diagnostic and monitoring methods provided herein further comprise selecting the one or more specific treatment regimens for the subject based on the results of the diagnostic and monitoring methods provided herein. In one embodiment, a treatment regimen known to be effective against Parkinson's disease having the marker signature detected in the subject/sample is selected for the subject. In certain embodiments, the treatment method is started, change, revised, or maintained based on the results from the diagnostic or prognostic methods of the invention, e.g., when it is determined that the subject is responding to the treatment regimen, or when it is determined that the subject is not responding to the treatment regimen, or when it is determined that the subject is insufficiently responding to the treatment regimen. In certain embodiments, the treatment method is changed based on the results from the diagnostic or prognostic methods.

In certain other embodiments the diagnostic and monitoring methods provided herein further comprise introducing one or more specific treatment regimens for the subject based on the results of the diagnostic and monitoring methods provided herein. In one embodiment, a treatment regimen known to be effective against Parkinson's disease is selected for the subject. In certain embodiments, the treatment method is started, change, revised, or maintained based on the results from the diagnostic or prognostic methods of the invention, e.g., when it is determined that the subject is responding to the treatment regimen, or when it is determined that the subject is not responding to the treatment regimen, or when it is determined that the subject is insufficiently responding to the treatment regimen. In certain embodiments, the treatment method is changed based on the results from the diagnostic or prognostic methods.

In yet other embodiments the diagnostic and monitoring methods provided herein further comprise the step of administering a therapeutically effective amount of an anti-Parkinson's disease therapy based on the results of the diagnostic and monitoring methods provided herein. In one embodiment, a treatment regimen known to be effective against Parkinson's disease is selected for the subject. In certain embodiments, the treatment method is administered based on the results from the diagnostic or prognostic methods of the invention, e.g., when it is determined that the subject expresses one or more markers of the invention (e.g., the markers NAP and/or EMA, specifically, or others in Table 2 and Table 5) above some threshold level that is indicative of Parkinson's disease.

In yet other embodiments the diagnostic and monitoring methods provided herein further comprise the step of administering a therapeutically effective amount of an anti-Parkinson's disease therapy based on the results of the diagnostic and monitoring methods provided herein. In one embodiment, a treatment regimen known to be effective against Parkinson's disease is selected for the subject. In certain embodiments, the treatment method is administered based on the results from the diagnostic or prognostic methods of the invention, e.g., when it is determined that the subject expresses one or more markers of the invention (e.g., the markers NAP and/or EMA, specifically, or others in Table 2 and Table 5) below some threshold level that is indicative of Parkinson's disease.

In yet other embodiments the diagnostic and monitoring methods provided herein further comprise the step of increasing, decreasing, or changing the dose of an anti-Parkinson's disease therapy based on the results of the diagnostic and monitoring methods provided herein. In one embodiment, a treatment regimen known to be effective against Parkinson's disease is selected for the subject. In certain embodiments, the treatment method is administered based on the results from the diagnostic or prognostic methods of the invention, e.g., when it is determined that the subject expresses one or more markers of the invention (e.g., the markers NAP and/or EMA, specifically, or others in Table 2 and Table 5) above some threshold level that is indicative of Parkinson's disease.

In yet other embodiments the diagnostic and monitoring methods provided herein further comprise the step of increasing, decreasing, or changing the dose of an anti-Parkinson's disease therapy based on the results of the diagnostic and monitoring methods provided herein. In one embodiment, a treatment regimen known to be effective against Parkinson's disease is selected for the subject. In certain embodiments, the treatment method is administered based on the results from the diagnostic or prognostic methods of the invention, e.g., when it is determined that the subject expresses one or more markers of the invention (e.g., the markers NAP and/or EMA, specifically, or others in Table 2 and Table 5) below some threshold level that is indicative of Parkinson's disease.

In certain embodiments of the diagnostic and monitoring methods provided herein, the method further comprises isolating a component of the biological sample, for example a protein.

In certain embodiments of the diagnostic and monitoring methods provided herein, the method further comprises labeling a component of the biological sample, for example a protein.

In certain embodiments of the diagnostic and monitoring methods provided herein, the method further comprises amplifying a component of a biological sample, for example a nucleic acid.

In certain embodiments of the diagnostic and monitoring methods provided herein, the method comprises forming a complex with a probe and a component of a biological sample. In certain embodiments, forming a complex with a probe comprises forming a complex with at least one non-naturally occurring reagent. In certain embodiments of the diagnostic and monitoring methods provided herein, the method comprises processing the biological sample. In certain embodiments of the diagnostic and monitoring methods provided herein, the method of detecting a level of at least two markers comprises a panel of markers. In certain embodiments of the diagnostic and monitoring methods provided herein, the method of detecting a level comprises attaching the marker to be detected to a solid surface.

The invention provides methods of selecting for administration of active treatment or against administration of active treatment of Parkinson's disease in a subject comprising:
(1) detecting a level of NAP and/or EMA, or that of one or more markers selected from the group consisting of the markers in Table 2 and Table 5 in a first sample obtained from the subject having Parkinson's disease at a first time wherein the subject has not been actively treated for Parkinson's disease;
(2) detecting a level of NAP and/or EMA, or that of one or more markers selected from the group consisting of the markers in Table 2 and Table 5 in a second sample obtained from the subject at a second time, e.g., wherein the subject has not been actively treated;
(3) comparing the level of NAP and/or EMA, or that of one or more markers selected from the group consisting of the markers in Table 2 and Table 5 in the first sample with the level of NAP and/or EMA, or that of one or more markers selected from the group consisting of the markers in Table 2 and Table 5 in the second sample;
wherein selecting for administration of active treatment or against administration of active treatment of Parkinson's disease is based on the presence or absence of changes in the level of the one or more markers between the first sample and the second sample.

In certain embodiments, the method further comprising obtaining a third sample obtained from the subject at a third time (e.g., wherein the subject has not been actively treated), detecting a level of NAP and/or EMA, or that of one or more markers selected from the group consisting of the markers in Table 2 and Table 5 in the third sample, and comparing the level of NAP and/or EMA, or that of one or more markers selected from the group consisting of the markers in Table 2 and Table 5 in the third sample with the level of NAP and/or EMA, or that of the one or more markers in the first sample and/or the one or more markers in the second sample.

In certain embodiments, a change in the level of the markers NAP and/or EMA, or one or more of the markers in Table 2 and Table 5 in the second sample as compared to the level of the markers in the first sample is an indication that the therapy is not efficacious in the treatment of Parkinson's disease. In particular embodiments, the change in the level of the markers is an increase in the level of the markers. In other embodiments, the change in the level of the markers is a decrease in the level of the markers.

In certain embodiments, a change in the level of the markers NAP and/or EMA, or one or more of the markers in Table 2 and Table 5 in the second sample as compared to the level of the markers in the first sample is an indication for selecting active treatment for Parkinson's disease. In particular embodiments, the change in the level of the markers is an increase in the level of the markers. In other embodiments, the change in the level of the markers is a decrease in the level of the markers.

In certain embodiments, no change in the level of expression of NAP and/or EMA, or one or more of the markers selected from the group consisting of the markers in Table 2 and Table 5 between the first sample and the second sample is an indication for selecting against active treatment for Parkinson's disease.

In certain embodiments, a change in the level of at least 2, 3, 4, 5, 6, 7, 8, 9, 10, 20, 30, 40, 50, 60, 70, 90, 100, 200, 300, 400, 500, 600, 700, 800, 900 or 1000 of the markers in Table 2 and Table 5 in the second sample as compared to the level of at least 2, 3, 4, 5, 6, 7, 8, 9, 10, 20, 30, 40, 50, 60, 70, 90, 100, 200, 300, 400, 500, 600, 700, 800, 900 or 1000 of the markers in Table 2 and Table 5 in the first sample has greater predictive value for selecting against active treatment for Parkinson's disease than analysis of a single marker alone.

### 6. Monitoring Clinical Trials

Monitoring the influence of agents (e.g., drug compounds) on the level of a marker of the invention can be applied not only in basic drug screening or monitoring the treatment of a single subject, but also in clinical trials. For example, the effectiveness of an agent to affect marker levels can be monitored in clinical trials of subjects receiving treatment for Parkinson's disease. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected markers of the invention (e.g., NAP and/or EMA, or one or more of the markers in Table 2 and Table 5) in the pre-administration sample; (iii) obtaining one or more post-administration samples from the subject; (iv) detecting the level of the marker(s) in the post-administration samples; (v) comparing the level of the marker(s) in the pre-administration sample with the level of the marker(s) in the post-administration sample or samples; and (vi) altering the administration of the agent to the subject accordingly. For example, an increase in the level of the marker during the course of treatment may indicate ineffective dosage and the desirability of increasing the dosage. In other embodiments, a decrease in the level of the marker during the course of treatment may indicate ineffective dosage and the desirability of increasing the dosage. Conversely, in some embodiments, a decrease in the level of the marker may indicate efficacious treatment and no need to change dosage. In other embodiments, an increase in the level of the marker may indicate efficacious treatment and no need to change dosage.

### H. Kits/Panels

The invention also provides compositions and kits for diagnosing, prognosing, or monitoring a disease or disorder, recurrence of a disorder, or survival of a subject being treated for a disorder (e.g., Parkinson's disease). These kits include one or more of the following: a detectable antibody that specifically binds to a marker of the invention, reagents for obtaining and/or preparing subject tissue samples for staining, and instructions for use.

The invention also encompasses kits for detecting the presence of a marker in a biological sample. Such kits can be used to determine if a subject is suffering from or is at increased risk of developing Parkinson's disease. For example, the kit can comprise a labeled compound or agent capable of detecting a marker in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for use of the kit for practicing any of the methods provided herein or interpreting the results obtained using the kit based on the teachings provided herein. The kits can also include reagents for detection of a control protein in the sample not related to Parkinson's disease, e.g., actin for tissue samples, albumin in blood or blood derived samples for normalization of the amount of the marker present in the sample. The kit can also include the purified marker for detection for use as a control or for quantitation of the assay performed with the kit. The kit can also include test materials for performing an anxiety test, a sleep test, a smell test, or any combination thereof, and optionally instructions for performing any one or more of the foregoing tests, as well as instructions or guidance for evaluating, and/or interpreting the results obtained.

Kits include a panel of reagents for use in a method to diagnose Parkinson's disease in a subject (or to identify a subject predisposed to developing Parkinson's disease, etc.), the panel comprising at least two detection reagents, wherein each detection reagent is specific for one Parkinson's disease-specific marker, wherein said Parkinson's disease-specific markers are selected from the Parkinson's disease-specific marker sets provided herein, such as, for example, NAP and/or EMA, or one or more of the markers of Table 2 or Table 5.

For antibody-based kits, the kit can comprise, for example: (1) a first antibody (e.g., attached to a solid support) which binds to a first marker; and, optionally, (2) a second, different antibody which binds to either the first marker or the first antibody and is conjugated to a detectable label. In certain embodiments, the kit includes (1) a second antibody (e.g., attached to a solid support) which binds to a second marker; and, optionally, (2) a second, different antibody which binds to either the second marker or the second antibody and is conjugated to a detectable label. The first and second markers are different. In an embodiment, the first and second markers are markers of the invention, e.g., NAP and/or EMA, or one or more of the markers in Table 2 and Table 5. In certain embodiments, the kit comprises a third antibody which binds to a third marker which is different from the first and second marker, and a second different antibody that binds to either the third marker or the antibody that binds the third marker wherein the third marker is different from the first and second marker.

For oligonucleotide-based kits, the kit can comprise, for example: (1) an oligonucleotide, e.g., a detectably labeled oligonucleotide, which hybridizes to a nucleic acid sequence encoding a marker protein or (2) a pair of primers useful for amplifying a marker nucleic acid molecule. In certain embodiments, the kit can further include, for example: (1) an oligonucleotide, e.g., a second detectably labeled oligonucleotide, which hybridizes to a nucleic acid sequence encoding a second marker protein or (2) a pair of primers useful for amplifying the second marker nucleic acid molecule. The first and second markers are different. In an embodiment, the first and second markers are markers of the invention, e.g., NAP and/or EMA, or one or more of the markers in Table 2 and Table 5. In certain embodiments, the kit can further include, for example: (1) an oligonucleotide, e.g., a third detectably labeled oligonucleotide, which hybridizes to a nucleic acid sequence encoding a third marker protein or (2) a pair of primers useful for amplifying the third marker nucleic acid molecule wherein the third marker is different from the first and second markers. In certain embodiments, the kit includes a third primer specific for each nucleic acid marker to allow for detection using quantitative PCR methods.

For chromatography methods, the kit can include markers, including labeled markers, to permit detection and identification of one or more markers of the invention, e.g., NAP and/or EMA, or one or more of the markers in Table 2 and Table 5, by chromatography. In certain embodiments, kits for chromatography methods include compounds for derivatization of one or more markers of the invention. In certain embodiments, kits for chromatography methods include columns for resolving the markers of the method.

Reagents specific for detection of a marker of the invention, e.g., NAP and/or EMA, or one or more of the markers in Table 2 and Table 5, allow for detection and quantitation of the marker in a complex mixture, e.g., plasma, serum, urine, or tissue sample. In certain embodiments, the reagents are species specific. In certain embodiments, the reagents are not species specific. In certain embodiments, the reagents are isoform specific. In certain embodiments, the reagents are not isoform specific.

In certain embodiments, the kits for the diagnosis, monitoring, or characterization of Parkinson's disease comprise at least one reagent specific for the detection of the level of NAP and/or EMA, or one or more of the markers selected from the group consisting of the markers in Table 2 and Table 5. In certain embodiments, the kits further comprise instructions for the diagnosis, monitoring, or characterization of Parkinson's disease based on the level of NAP and/or EMA, or one or more of the markers selected from the group consisting of the markers in Table 2 and Table 5.

In certain embodiments, the kits can also comprise, e.g., a buffering agent, a preservative, a protein stabilizing agent, or a reaction buffer. The kit can further comprise components necessary for detecting the detectable label (e.g., an enzyme or a substrate). The kit can also contain a control sample or a series of control samples that can be assayed and compared to the test sample. The controls can be control serum samples or control samples of purified proteins or nucleic acids, as appropriate, with known levels of target markers. Each component of the kit can be enclosed within an individual container and all of the various containers can be within a single package, along with instructions for interpreting the results of the assays performed using the kit.

The kits of the invention may optionally comprise additional components useful for performing the methods of the invention.

The invention further provides panels of reagents for detection of one or more markers of Parkinson's disease in a subject sample and at least one control reagent. In certain embodiments, the control reagent is to detect the marker for detection in the biological sample wherein the panel is provided with a control sample containing the marker for use as a positive control and optionally to quantitate the amount of marker present in the biological sample. In certain embodiments, the panel includes a detection reagent for a maker not related to Parkinson's disease that is known to be present or absent in the biological sample to provide a positive or negative control, respectively. The panel can be provided with reagents for detection of a control marker in the sample not related to Parkinson's disease, e.g., actin for tissue samples, albumin in blood or blood derived samples for normalization of the amount of the marker present in the sample. The panel can be provided with a purified marker for detection for use as a control or for quantitation of the assay performed with the panel.

In a preferred embodiment, the panel includes reagents for detection of two or more markers of the invention (e.g., 2, 3, 4, 5, 6, 7, 8, 9, 10, 11, 12, 13, 14, 15, 16, 17, 18, 19, 20, 21, 22, 23, 24, or 25), preferably in conjunction with a control reagent. In the panel, each marker is detected by a reagent specific for that marker. In certain embodiments, the panel includes replicate wells, spots, or portions to allow for analysis of various dilutions (e.g., serial dilutions) of biological samples and control samples. In a preferred embodiment, the panel allows for quantitative detection of one or more markers of the invention.

In certain embodiments, the panel is a protein chip for detection of one or more markers. In certain embodiments, the panel is an ELISA plate for detection of one or more markers. In certain embodiments, the panel is a plate for quantitative PCR for detection of one or more markers.

In certain embodiments, the panel of detection reagents is provided on a single device including a detection reagent for one or more markers of the invention and at least one control sample. In certain embodiments, the panel of detection reagents is provided on a single device including a detection reagent for two or more markers of the invention and at least one control sample. In certain embodiments, multiple panels for the detection of different markers of the invention are provided with at least one uniform control sample to facilitate comparison of results between panels.

This invention is further illustrated by the following examples which should not be construed as limiting. The contents of all references and published patents and patent applications cited throughout the application are hereby incorporated by reference.

### EXAMPLES

This invention is further illustrated by the following examples which should not be construed as limiting. The contents of all references, GenBank Accession and Gene numbers, and published patents and patent applications cited throughout the application are hereby incorporated by reference. Those skilled in the art will recognize that the invention may be practiced with variations on the disclosed structures, materials, compositions and methods, and such variations are regarded as within the ambit of the invention.

### Example 1: Identification of PD Markers in Samples from PD Patients and Control Subjects

This Example describes a high confidence subset of biomarkers based on both network analysis and statistical analysis. The samples analyzed were plasma samples from patients with Parkinson's disease and from control subjects that were not afflicted with Parkinson's disease. The sample counts of Parkinson's disease patients and control subjects is set forth in Table 1.

TABLE 1

| Sample Counts | | Batch 1 | Batch 2 | Totals |
|---|---|---|---|---|
| PD | Male | 83 | 30 | 113 |
| | Female | 64 | 19 | 83 |
| Control | Male | 49 | 63 | 112 |
| | Female | 52 | 32 | 84 |

| | | | |
|---|---|---|---|
| Totals | 246 | 146 | 392 |

The samples were subjected to proteomic, lipidomics and metabolics analysis as described below.

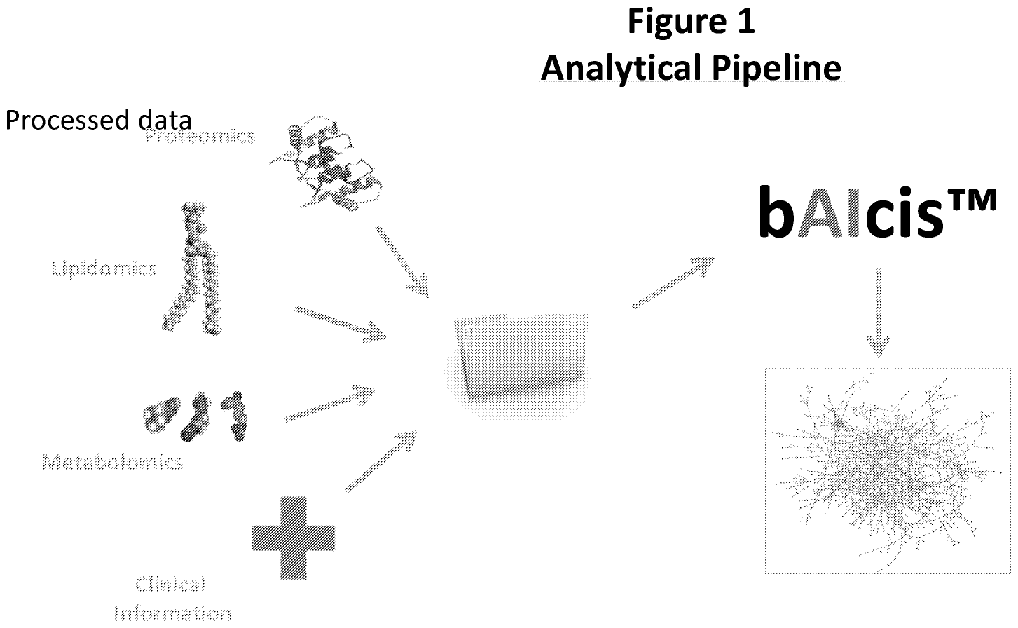

An interrogative systems biology based discovery platform (i.e., bAIcis™) was used to obtain mechanistic insights into understanding the role of the analyzed proteins, lipids and/or metabolites in PD (FIG. 1). The Platform technology, which is based on the methodology described in detail in WO2012119129, involves discovery across a hierarchy of systems including human plasma samples from PD patients and downstream data integration and mathematical modeling employing an Artificial Intelligence (AI) based informatics module.

### Overview of Biomarker Selection Process

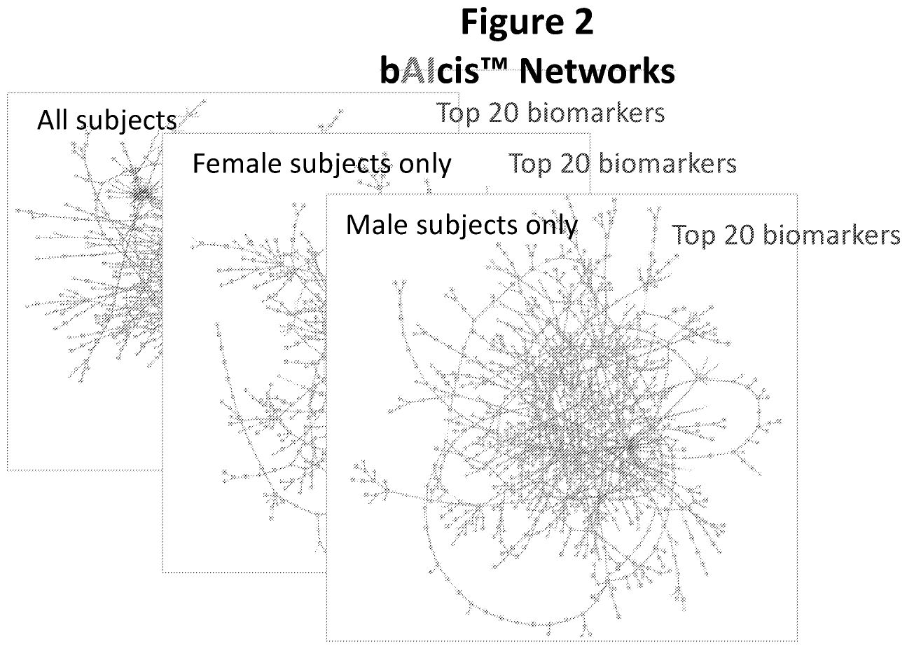

Biomarkers were selected in 4 different methods either based on statistical or network analysis. First, bAIcis™ networks were built for all omics and clinical data (e.g., Hoehn-Yahr scale stages, PD history, and demographics) from both batches. In particular, bAIcis networks were built for all subjects (i.e., female and male), female subjects only, and male subjects only (FIG. 2). Biomarkers were identified based on topology from the following delta networks: PD—normal; PD—normal, male subjects only; and PD—normal, female subjects only.

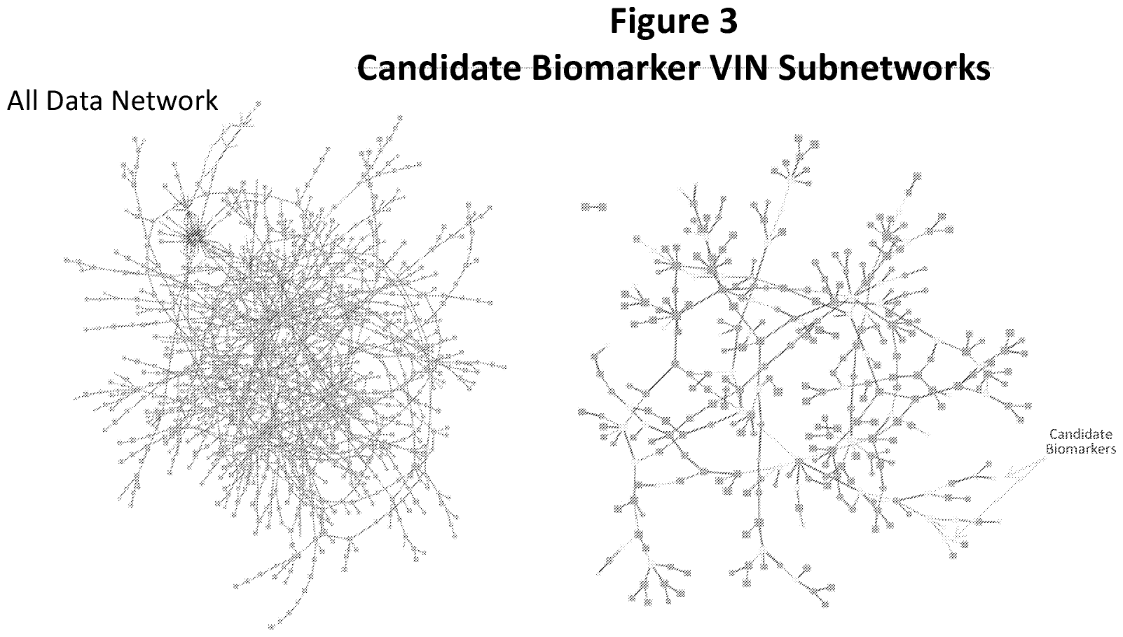

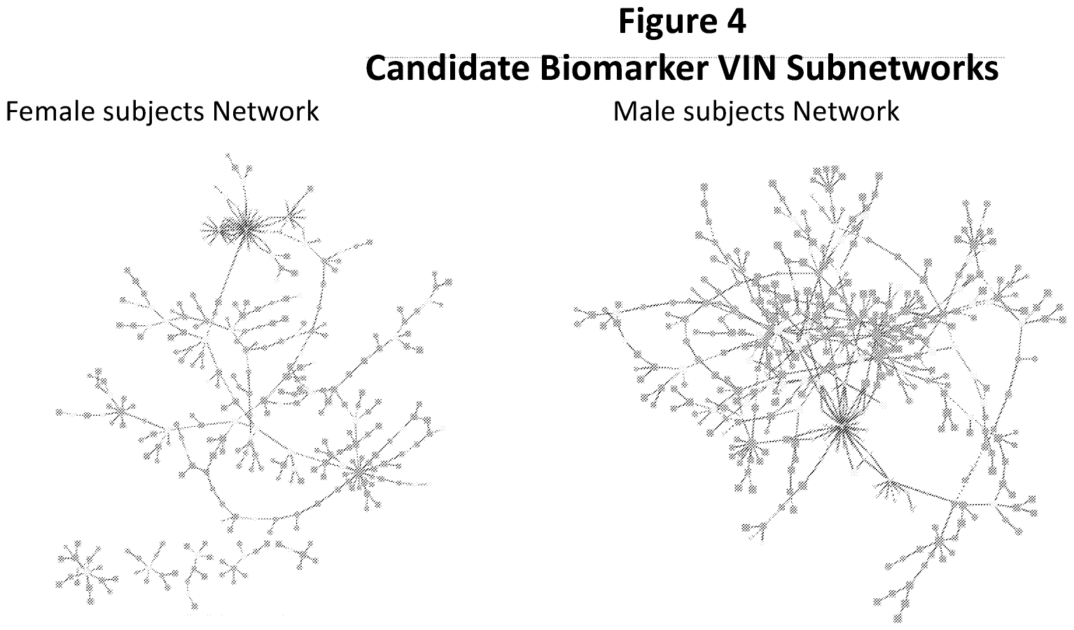

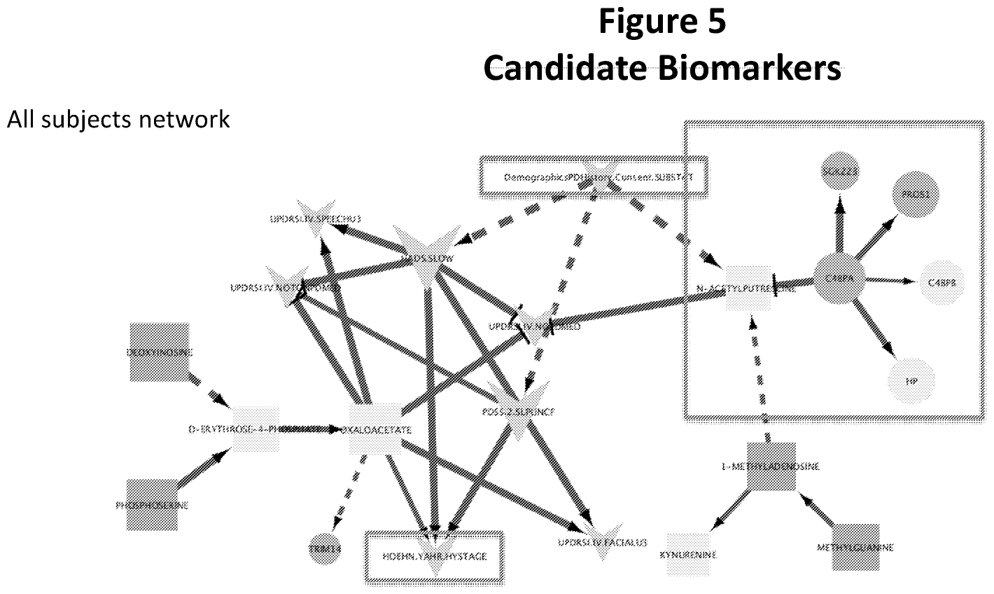

VIN subnetworks for all data (FIG. 3) and female subjects and male subjects (FIG. 4) were created from the three delta networks listed above by identifying first and second degree neighbors of clinical variables corresponding to disease status and PD staging. Biomarkers identified based on this analysis are shown in FIG. 5, and include deoxyinosine, phosphoserine, 1-methyladenosine, methylguanine, TRIM14, SGK223, PROS1, C4BPA, C4BPB, HP, D-erythrose-4-phosphate, oxaloacetate/methysuccinate/ethylmalonic acid/glutaric acid, N-acetylputrescine, and kynurenine (protein markers are indicated by circles).

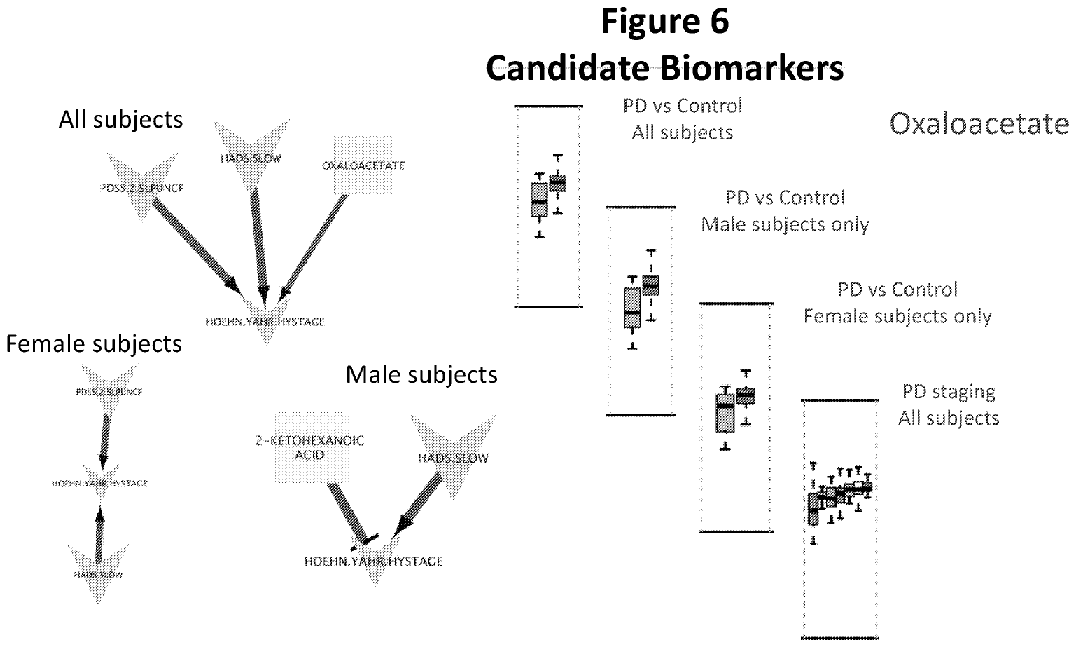

Box plots depicting biomarker oxaloacetate/methysuccinate/ethylmalonic acid/glutaric acid (referred to oxaloacetate) are shown in FIG. 6 for PD vs. control for all subjects, PD vs. control for male subjects only, and PD vs. control for female subjects only. For all analysis, oxaloacetate/methysuccinate/ethylmalonic acid/glutaric acid is increased in PD vs. control samples. PD staging based on Hoehn-Yahr scale stages 1.0, 1.5, 2.0, 2.5, 3, and 4 for all subjects is also depicted. The control for each analysis is depicted on the left.

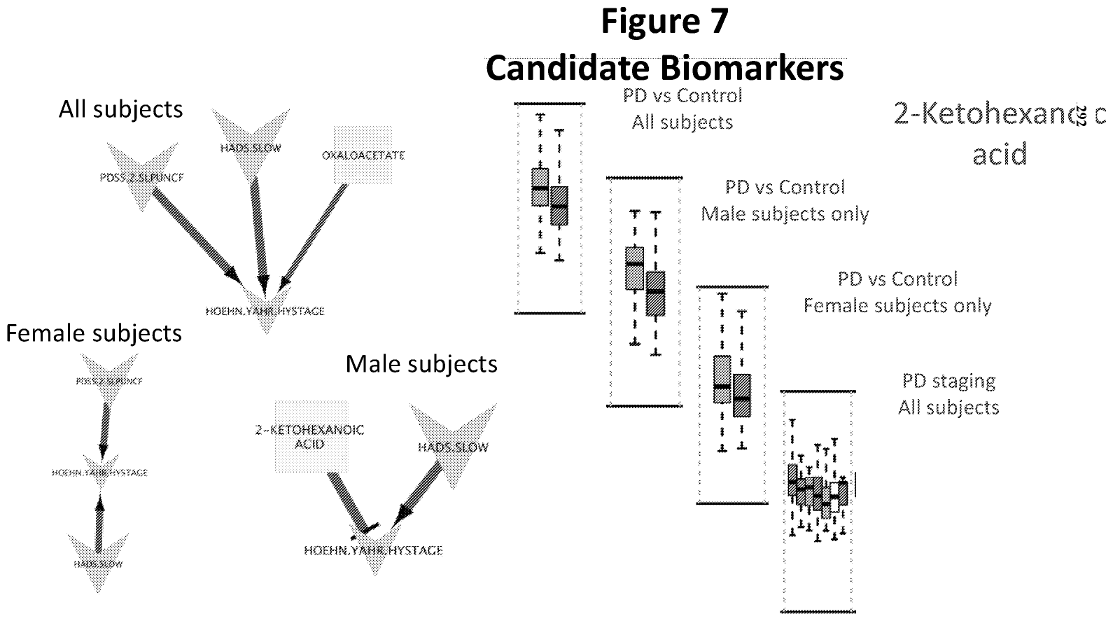

Box plots depicting biomarker 2-ketohexanoic acid are shown in FIG. 7 for PD vs. control for all subjects, PD vs. control for male subjects only, and PD vs. control for female subjects only. For all analysis, 2-ketohexanoic acid is decreased in PD vs. control samples. PD staging based on Hoehn-Yahr scale stages 1.0, 1.5, 2.0, 2.5, 3, and 4 for all subjects is also depicted. The control for each analysis is depicted on the left.

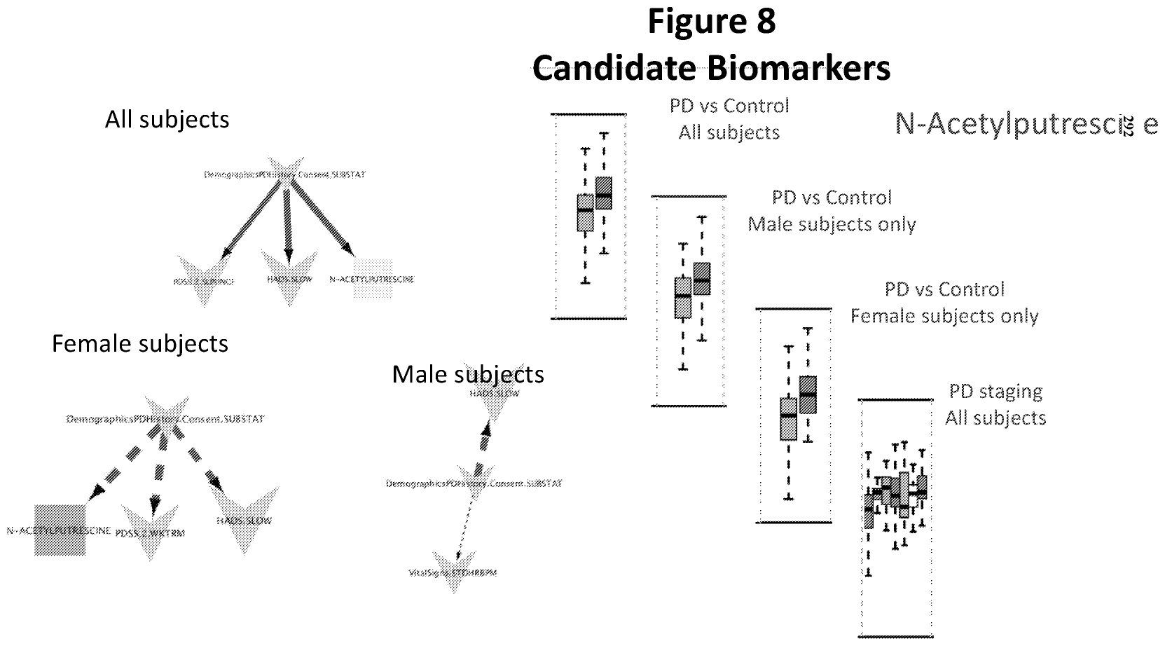

Box plots depicting biomarker N-acetylputrescine are shown in FIG. 8 for PD vs. control for all subjects, PD vs. control for male subjects only, and PD vs. control for female subjects only. For all analysis, N-acetylputrescine is increased in PD vs. control samples. PD staging based on Hoehn-Yahr scale stages 1.0, 1.5, 2.0, 2.5, 3, and 4 for all subjects is also depicted. The control for each analysis is depicted on the left.

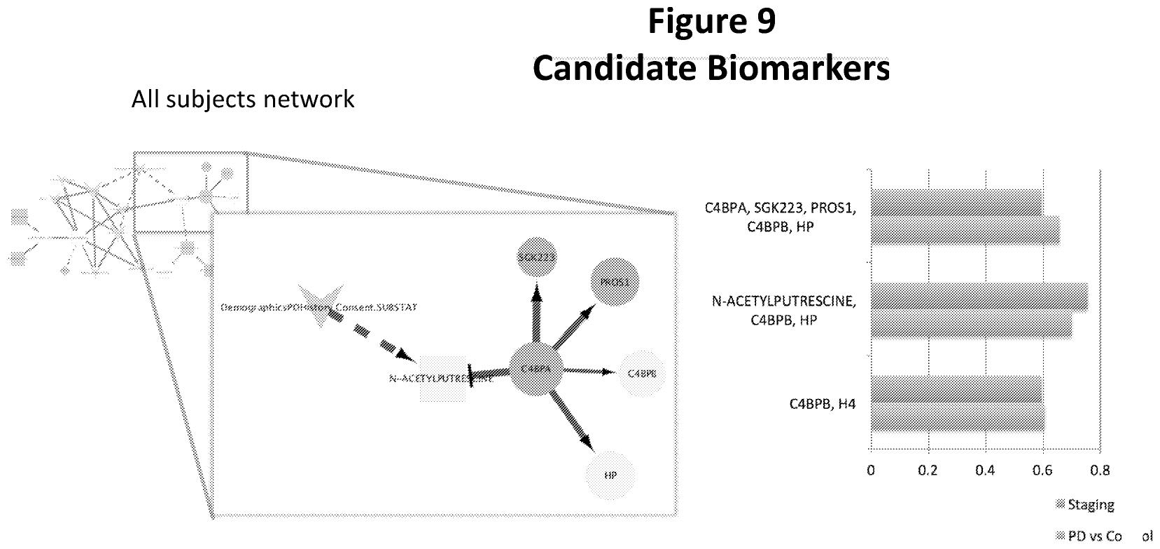

FIG. 9 depicts staging (based on the Hoehn-Yahr scale) and PD vs. control for the “all subjects network” for various combinations of biomarkers N-acetylputrescine, C4BPA, C4BPB, SGK223, HP, and PROS1.

Table 2 includes certain markers identified based on the network and VIN analysis as described above.

TABLE 2

oxaloacetate/methysuccinate/ethylmalonic acid/
glutaric acid
N-acetylputrescine
2-ketohexanoic acid
D-erythrose-4-phosphate
kynurenine
methylguanine
1-methyladenosine
phosphoserine
deoxyinosine
TRIM14 (Tripartite Motif Containing 14)
SGK223 (Tyrosine-Protein Kinase SgK223)
PROS1 (Protein S (Alpha))
C4BPA (Complement Component 4 Binding Protein Alpha)
C4BPB (Complement Component 4 Binding Protein Beta)
HP (Haptoglobin)

A batch analysis list was generated based on overlap of markers identified from batch 1 only and batch 2 only analysis. For each batch, top 50 markers in each omics category were chosen using limma (glmnet package in R). Markers that appeared in both lists were retained.

For permutation analysis, all data was used. Markers that were present in a certain number of permutations were selected.

Table 3, below, shows the number of markers selected from each omics type through different analysis methods. ‘Network’ includes biomarkers identified through network topology of delta networks. ‘VIN’ includes first and second degree neighbors of clinical variables for PD diagnosis and staging. ‘Batch analysis’ is based on overlap of markers selected in the batch 1 and batch 2 analysis. ‘Permutation analysis’ includes biomarkers selected based on permutation analysis using all samples.

TABLE 3

| | | | | Permutation |
|---|---|---|---|---|
| Omics type | Network | VIN | Batch analysis | analysis |
| Proteomics | 20 | 2 | 33 | 16 |
| Signalling | 1 | 0 | 10 | 5 |
| lipidomics | | | | |
| Structural | 61 | 0 | 23 | 0 |
| lipidomics | | | | |
| Metabolomics | 23 | 4 | 40 | 19 |
| TOTAL | 105 | 6 | 106 | 40 |

### High Confidence Marker Analysis

High confidence markers were identified as markers that were present in at least one network based method and one statistics based method, as described above. Table 4 shows the 9 markers that were selected by this process. For all 9 markers, box plots showing data are shown in FIG. 10A-D and FIG. 11A-D.

TABLE 4
Analyses in which markers were identified.

| | | | | | Permutation |
|---|---|---|---|---|---|
| | Biomarker | Network | VIN | Batch analysis | analysis |
| 1. | P13591 | PD-control | | PD vs control | |
| | (NCAM) | with all data | | with all data | |
| | | (outdegree) | | | |
| 2. | SL-9-HODE | PD-control | | All models | |
| | | with all data | | | |
| | | (outdegree) | | | |
| 3. | BM000397 (N- | | X | PD vs control | All models |
| | acetyl- | | | with all data | |
| | puterscine) | | | PD vs control | |
| | | | | with male data | |
| | | | | Staging model | |
| 4. | oxaloacetate/ | | X | All models | PD vs control |
| | methysuccinate/ | | | | with male |
| | ethylmalonic | | | | data |
| | acid/glutaric | | | | PD vs control |
| | acid | | | | with female |
| | | | | | data |
| | | | | | Staging model |
| 5. | Q14624.3 | PD-control | | PD vs control | |
| | (ITIH4) | with all data | | with male data | |
| | | (outdegree) | | PD vs control | |
| | | | | with female | |
| | | | | data | |
| 6. | F5GZZ9 | PD-control | | PD vs control | |
| | (CD163) | with all data | | with male data | |
| | | (outdegree) | | | |
| 7. | AC-10:2 | PD-control | | PD vs control | |
| | | with all data | | with male data | |
| | | (outdegree) | | | |
| 8. | AC-10:3 | PD-control | | PD vs control | |
| | | with all data | | with male data | |
| | | (outdegree) | | | |
| 9. | PE-36:6 | PD-control | | Staging model | |
| | | with all data | | | |
| | | (outdegree) | | | |

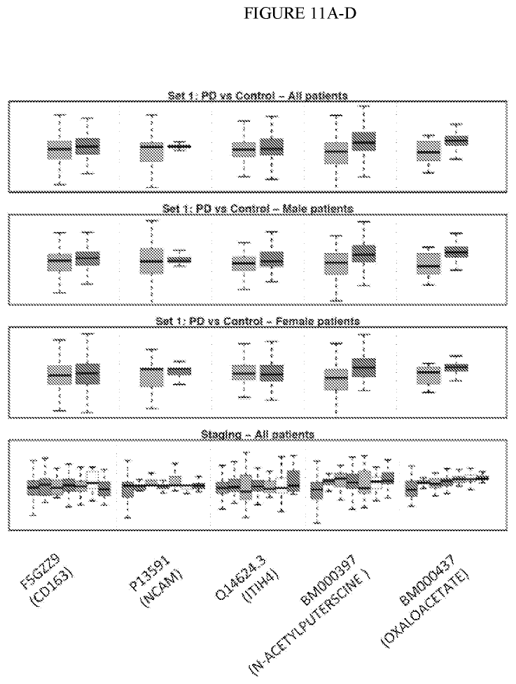

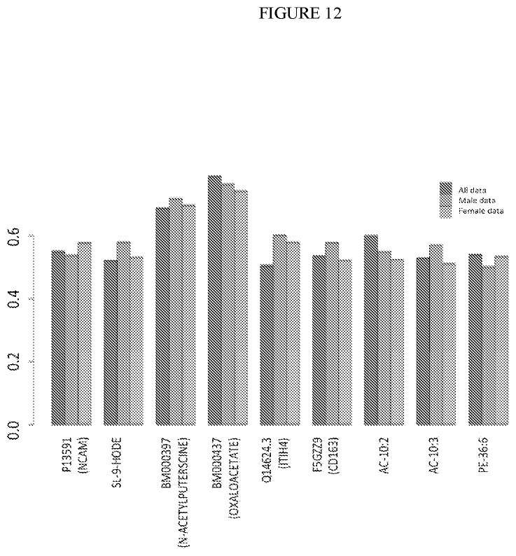

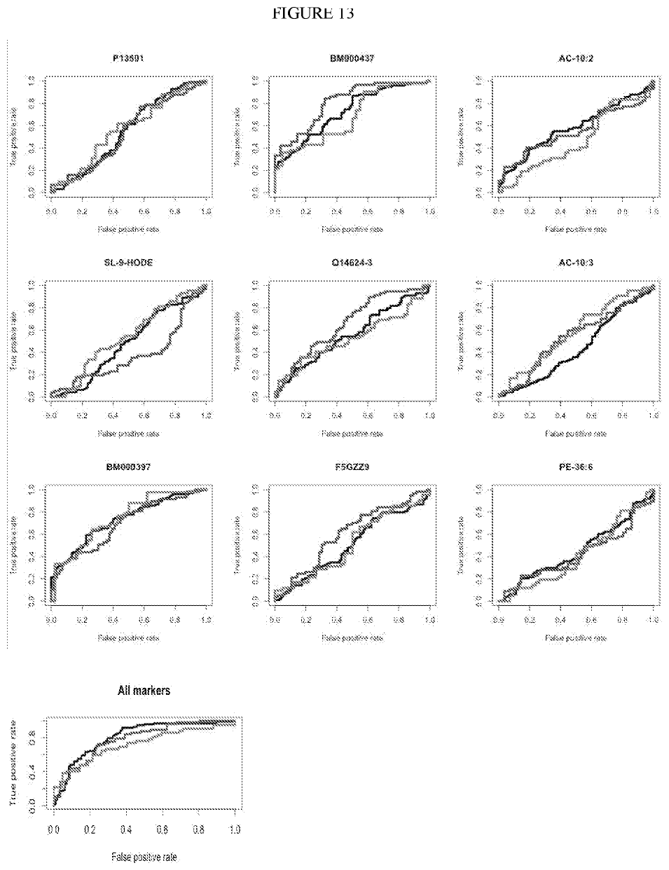

AUCs were calculated for PD vs Control by using 50% of the data as the training data and 50% of the data as the testing data. Results from AUC calculations are shown in Table 5 and FIG. 12. FIG. 13 shows corresponding ROC curves. In addition to one marker models, a model containing all 9 markers was built.

TABLE 5
AUCs for PD vs control models for selected biomarkers. All
marker model includes all 9 selected markers.

| Markers | All data | Male | Female |
|---|---|---|---|
| P13591 (NCAM) | 0.55 | 0.54 | 0.58 |
| SL-9-HODE | 0.52 | 0.58 | 0.53 |
| BM000397 (N-acetylputerscine) | 0.69 | 0.72 | 0.70 |
| oxaloacetate/ | 0.79 | 0.76 | 0.74 |
| methysuccinate/ | | | |
| ethylmalonic acid/glutaric acid | | | |
| Q14624.3 (ITIH4) | 0.51 | 0.60 | 0.58 |
| F5GZZ9 (CD163) | 0.54 | 0.58 | 0.52 |
| AC-10:2 | 0.60 | 0.55 | 0.52 |
| AC-10:3 | 0.53 | 0.57 | 0.51 |
| PE-36:6 | 0.54 | 0.50 | 0.53 |
| All markers | 0.81 | 0.78 | 0.72 |

It was noted that multiple isobaric metabolites were detected in human bio-fluids which may correspond to the metabolite oxaloacetate.” In particular, oxaloacetic acid (oxaloacetate), methylsuccinic acid (methylsuccinate), ethylmalonic acid and glutaric acid were all found to fit the parameters for oxaloacetate detection and measurement using an HILIC-LS-MS/MS system. In other words, no separation between oxaloacetic acid, methylsuccinic acid, ethylmalonic acid and glutaric acid was observed in an HELIC-LS-MS/MS mass chromatogram (see FIG. 15), and thus the markers oxaloacetate, methysuccinate, ethylmalonic acid and glutaric acid are not distinguishable using that method. Therefore, the marker identified herein as oxaloacetate/methysuccinate/ethylmalonic acid/glutaric acid” refers to all of the markers oxaloacetic acid (oxaloacetate), methylsuccinic acid (methylsuccinate), ethylmalonic acid and glutaric acid. In addition, the biomarkers identified as oxaloacetic acid (oxaloacetate), methylsuccinic acid (methylsuccinate), ethylmalonic acid and glutaric acid are used interchangeably herein to refer to any one of these biomarkers.

In one embodiment, these markers may be detected and used in the methods of the invention separately from each other using methods known in the art. In another embodiment, two, three, or four of these markers may be used in combination. In a preferred embodiment, methylsuccinate is detected separately and used in the methods of the invention.

To account for staging, subjects were divided based on stage and AUC calculations were made. Table 6 shows the amount of data available for various stages. Stages are based on the Hoehn-Yahr scale, which is a commonly used system for describing how the symptoms of Parkinson's disease progress. The scale allocates stages from 0 to 5 to indicate the relative level of disability:
Stage 0: No signs of disease
Stage 1.0: Symptoms are very mild; unilateral involvement only
Stage 1.5: Unilateral and axial involvement
Stage 2: Bilateral involvement without impairment of balance
Stage 2.5: Mild bilateral disease with recovery on pull test
Stage 3: Mild to moderate bilateral disease; some postural instability; physically independent
Stage 4: Severe disability; still able to walk or stand unassisted
Stage 5: Wheelchair bound or bedridden unless aided

TABLE 6
Number of subjects based on gender and PD stage assignments.

| Staging | All subjects | Male subjects | Female subjects |
|---|---|---|---|
| 0 | 196 | 114 | 85 |
| 1 | 21 | 7 | 14 |
| 1.5 | 22 | 9 | 14 |
| 2 | 86 | 57 | 31 |
| 2.5 | 38 | 25 | 14 |
| 3 | 21 | 15 | 7 |
| 4 | 7 | 3 | 4 |
| 5 | 1 | 1 | 0 |

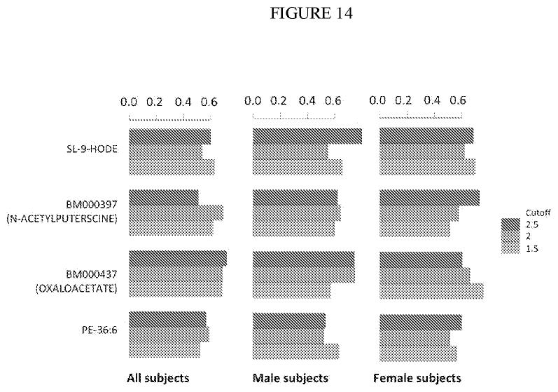

All patients with stage assignment of 0 were removed from analysis. Three different thresholds 1.5, 2 and 2.5 were selected to divide subjects in 2 groups for each threshold. For example, for a threshold of 1.5 the two groups are: (1, 1.5) and (2, 2.5, 3, 4, 5). Regression models were built to separate the 2 groups for each of the thresholds. AUC values are shown in FIG. 14 and Table 7.

TABLE 7
Shows AUC values corresponding to FIG. 14.

| | Molecule | | | | | | | | |
|---|---|---|---|---|---|---|---|---|---|
| | Cutoff - 2.5 | | | Cutoff - 2 | | | Cutoff - 1.5 | | |
| Subjects | All | Male | Female | All | Male | Female | All | Male | Female |
| SL-9-HODE | 0.59 | 0.80 | 0.80 | 0.53 | 0.55 | 0.61 | 0.62 | 0.66 | 0.69 |
| BM000397 - | 0.50 | 0.62 | 0.72 | 0.68 | 0.64 | 0.57 | 0.61 | 0.60 | 0.51 |
| N- | | | | | | | | | |
| acetylputerscine | | | | | | | | | |
| BM000437 - | 0.70 | 0.74 | 0.59 | 0.68 | 0.75 | 0.65 | 0.68 | 0.57 | 0.74 |
| oxaloacetate/ | | | | | | | | | |
| methysuccinate/ | | | | | | | | | |
| ethylmalonic | | | | | | | | | |
| acid/glutaric | | | | | | | | | |
| acid | | | | | | | | | |
| PE-36:6 | 0.56 | 0.53 | 0.59 | 0.58 | 0.52 | 0.51 | 0.51 | 0.63 | 0.55 |

### Example 2: Metabolic Stability Assessment of Parkinson's Disease Biomarkers

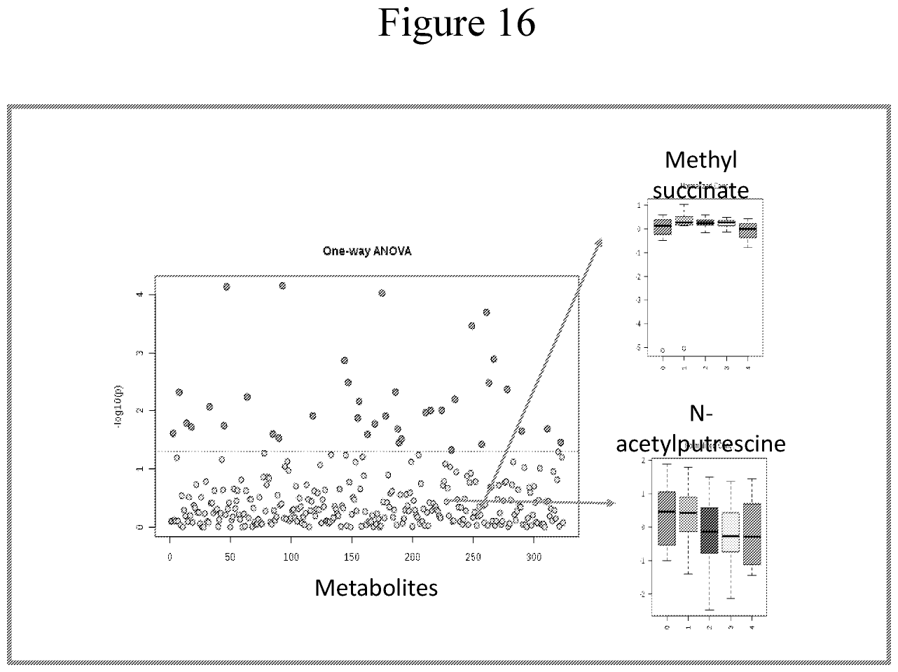

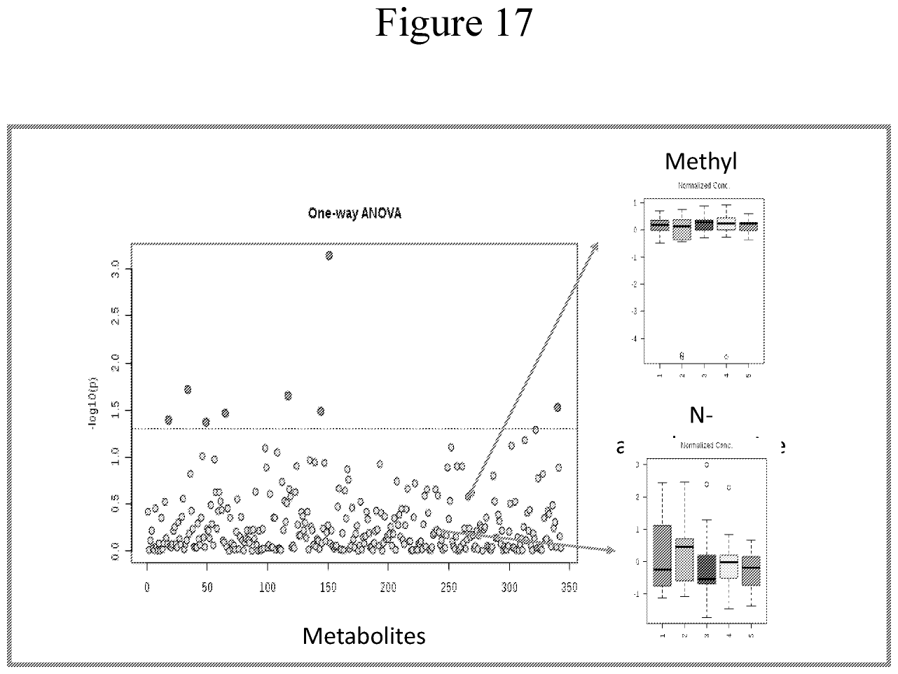

An important aspect of biomarker discovery is to ensure that the stability of a biomarker does not change naturally with diet, over the course of the day, or over several days (e.g., based on circadian rhythm). In order to assess the metabolic stability of certain Parkinson's disease biomarkers, an experiment was performed wherein the concentrations of 350 metabolites, including methylsuccinate and N-acetylputerscine, were monitored throughout the day at five time points (i.e., 7 AM, 10 AM, 1 PM, 4 PM, and 7 PM) in healthy control subjects, without fasting (see FIG. 16). In addition, the metabolites were also monitored over five different days (Monday through Friday) in healthy control subjects, with fasting conditions (monitoring was performed at 7 AM with no prior food intake that day) (see FIG. 17).

As set forth in FIGS. 16 and 17, the lighter gray dots (below the line) represent metabolites that do not change in a statistically significant manner, do not change with fasting, and do not have a lot of variation during the day (FIG. 16) or over several days (FIG. 17). The darker dots (above the line) represent metabolites that do change in a statistical manner during the day (FIG. 16) or over several days (FIG. 17) and therefore do not represent ideal biomarkers.

As set forth in FIG. 16, biomarkers methylsuccinate and N-acetylputerscine are metabolites that did not change over the course of the day. As set forth in FIG. 17, methylsuccinate and N-acetylputerscine are metabolites that did not change over the course of five days. Thus, these biomarkers are metabolically stable and useful in the detection of Parkinson's disease.

### Example 3: Analysis of Concomitant Medications on Parkinson's Disease Biomarkers

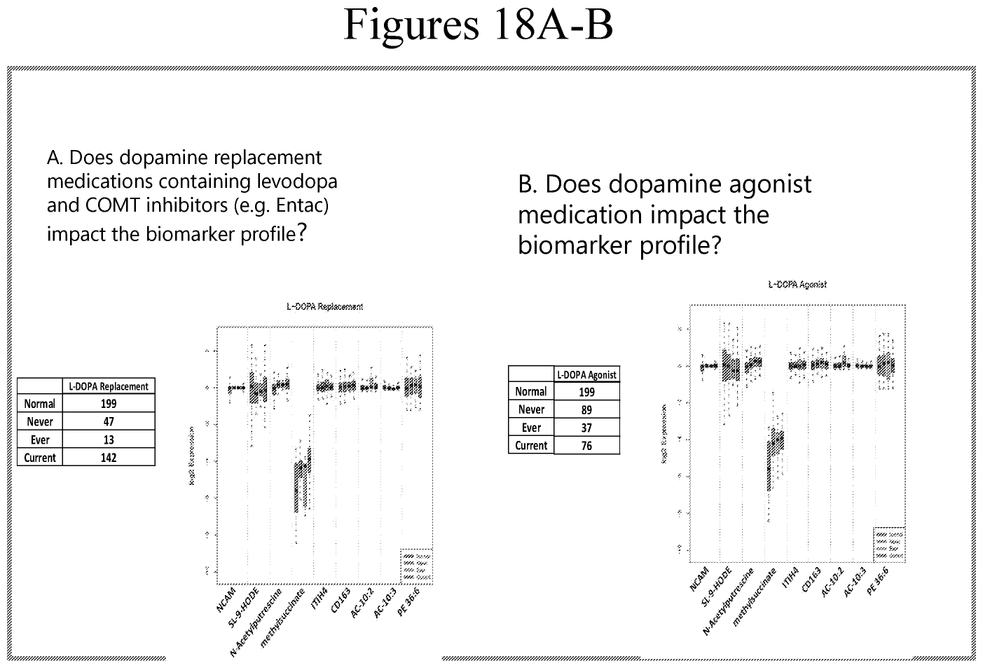

This example describes the effect of concomitant medications on PD biomarkers P13591 (NCAM), SL-9-HODE, methysuccinate, N-acetylputerscine, Q14624.3 (MH4), F5GZZ9 (CD163), AC-10:2, AC-10:3, and PE-36:6. Concentrations of each marker were measured in the following patients: non-diseased controls, individuals without PD (“Normal”); PD patients that have never been exposed to the drug (“Never”), PD patients that at one time were exposed to the drug (“Ever”); and PD patients that are currently taking the drug (“Current”). The numbers of patients tested in each category are set forth in tables in FIGS. 18A-B, 19A-B, and 20A-B.

FIG. 18A depicts the impact of dopamine replacement medications containing levodopa and COMT inhibitors (e.g., Entac) on the biomarkers. FIG. 18B depicts the impact of dopamine agonist medication on the biomarkers.

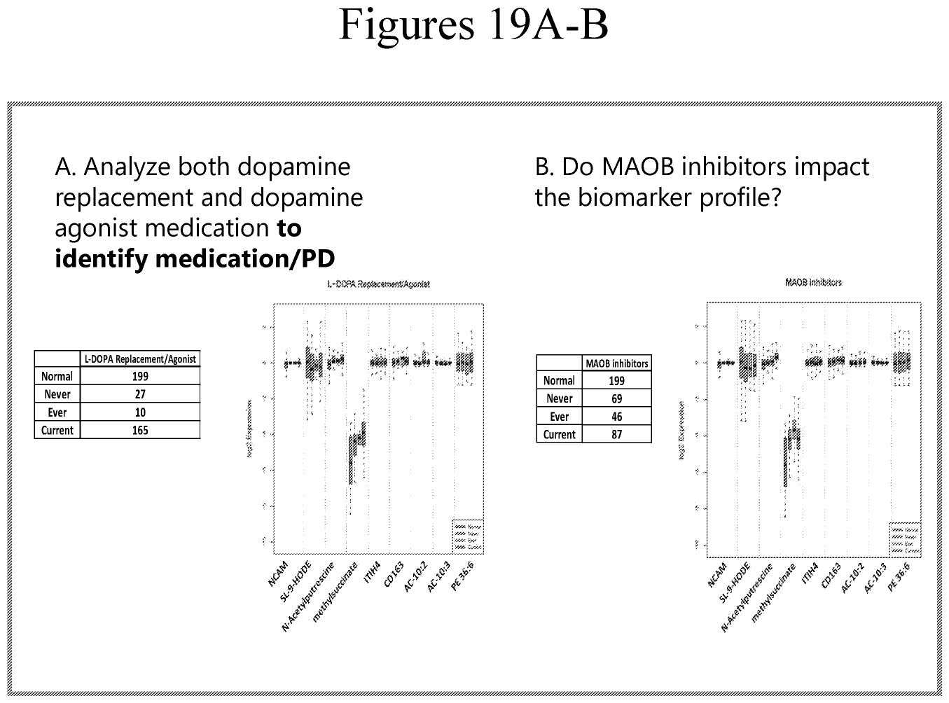

FIG. 19A depicts the impact of both dopamine replacement and dopamine agonist medication on the biomarkers. FIG. 19B depicts the impact of MAOB inhibitors on the biomarkers.

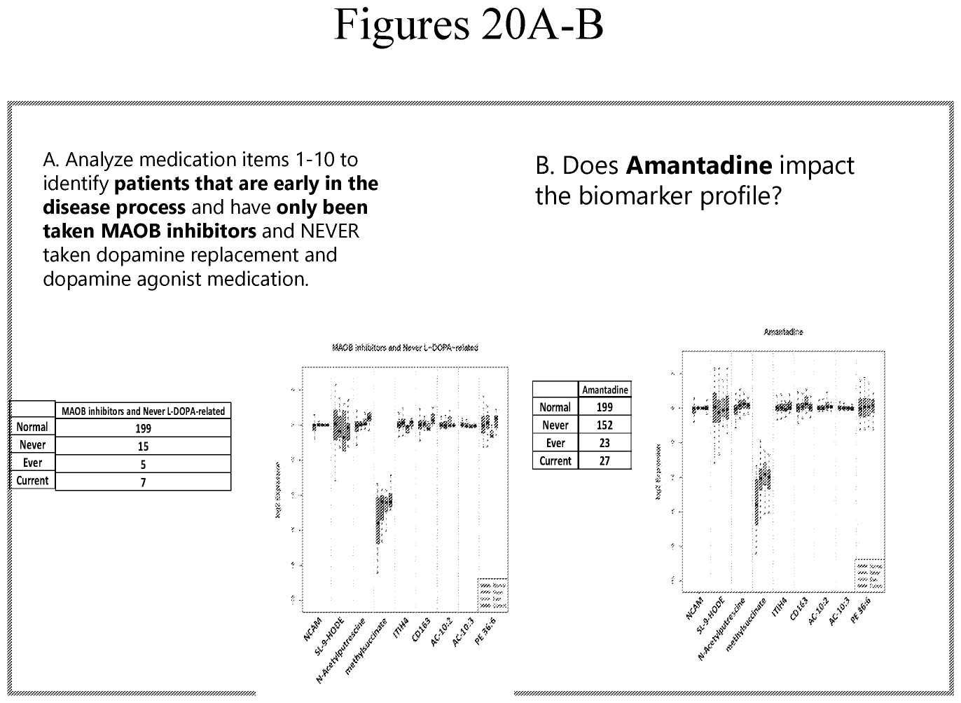

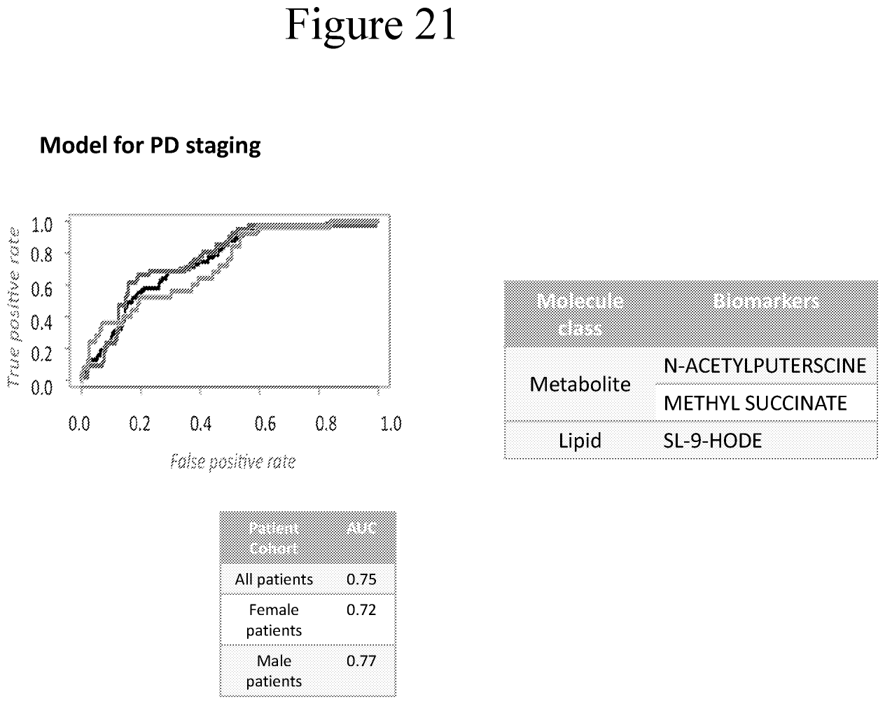

FIG. 20A depicts the impact on the biomarkers in patients that are early in the disease process, have only taken MAOB inhibitors and have never taken dopamine replacement or dopamine agonist medication. FIG. 20B depicts the impact of Amantadine, an antiparkinsonian drug, on the biomarkers.

The results of these studies indicate that no one drug affects the biomarker profiles of the biomarkers tested.

### Example 4: Multi-Omics Biomarkers Panel Combination with Clinical Features

This example describes the use of metabolite biomarkers in combination with clinical features to diagnose PD. AUCs were calculated for subjects having PD vs. Control.

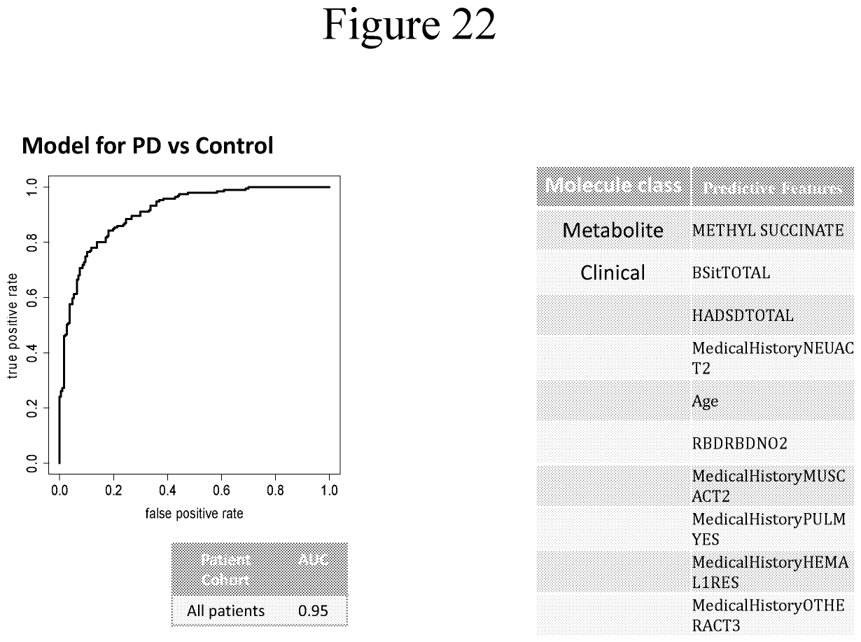

As set forth in FIG. 22, an AUC of 0.95 was obtained for all patients with biomarker methylsuccinate in combination with several clinical features including BSitTotal—combined score from the smell test; HADSDTotal—Total Depression Score; MedicalHistory NeuACT2—Neurologic Condition 2 Active; Age—age; RBDRBDNO2—not acting out dreams while asleep; MedicalHistoryMUSCAT2—Musculoskeletal Condition 2 Active; MedicalHistoryPULMYES—Pulmonary condition; MedicalHistoryHEMAL1RES—Hematolymphatic Condition 1 Resolved; and MedicalHistoryOTHERACT3—OTHER condition 3 Active.

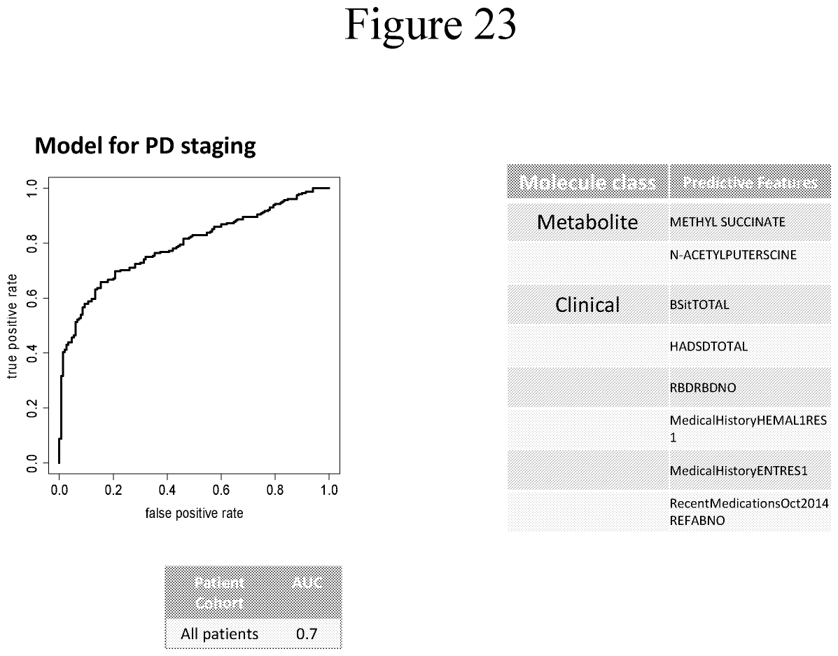

As set forth in FIG. 23, an AUC of 0.70 was obtained for all patients with biomarkers methylsuccinate and N-acetylputrescine in combination with several clinical features including BSitTotal—combined score from the smell test; HADSDTotal—Total Depression Score; RBDRBDNO2—not acting out dreams while asleep; MedicalHistoryHEMAL1RES—Hematolymphatic Condition 1 Resolved. MedicalHistoryENTRES1—ENT Condition 1 Resolved.

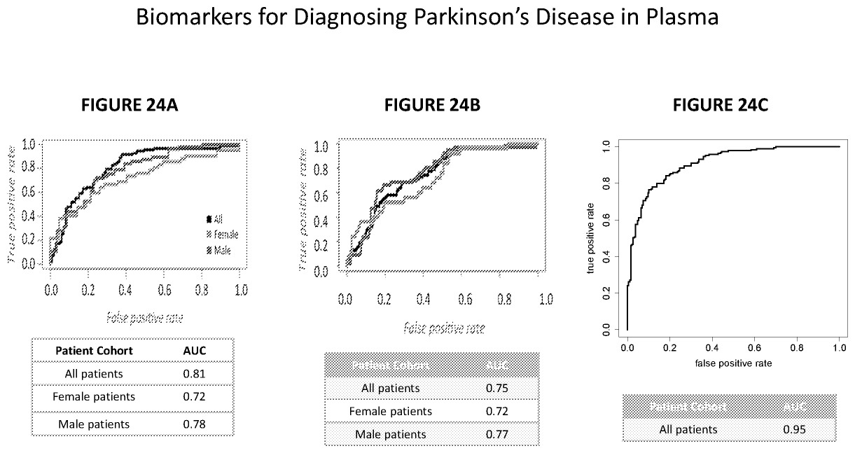

FIG. 24A represents the best combination of markers distinguishing parkinsons disease from non parkinsons disease patients. Further, the optimal combination of markers for male as well as female were identified for parkinsons disease vs non-disease patients. The performance of these markers was assessed by receiver operator control analysis.

### Example 5: LC-MS/MS Method for Detecting Plasma Biomarkers for Parkinson's Disease

Liquid chromatograph-mass spectrometry (LC-MS/MS) or liquid chromatography with tandem mass spectrometry were developed for detecting and quantitating six plasma biomarkers of Parkinson's Disease (PD): Ethyl malonic acid (EMA), Glutaric acid (GA), Methylsuccinic acid (MSA), and N-acetyl putrescine (NAP) identified in a metabolomics study, as well as Neural Cell Adhesion Molecule (NCAM or CD56) and Inter-alpha-trypsin inhibitor heavy chain family member 4 (ITIH4) identified in a proteomic study.

NAP is the N-acetylated form of the naturally occurring polyamine putrescine. MSA is a small dicarboxylic acid metabolite found in human biofluids associated with ethylmalonic encephalopathy, isovaleric acidemia, and medium chain acyl-CoA dehydrogenase deficiency. GA and EMA are positional isomers of MSA with the same molecular weight that may also be present in plasma.

ITIH4 is a plasma serine protease inhibitor involved in extracellular matrix stabilization and in prevention of tumor metastasis. NCAM-1 is a hemophilic glycoprotein that is a member of the immunoglobulin family and plays an important role in the development of the nervous system.

Detection and Quantitation of MSA/EMA/GA Using LC-MS/MS. MSA, EMA and GA isomers were detected and quantitated as follows.

### Materials

2-methylsuccinic acid, ethylmalonic acid, glutaric acid, formic acid and SigMatrix ultra serum diluent were purchased from Sigma-Aldrich (St. Louis, MO, USA). 2-methylsuccinic acid-d6 (purity: 99%) and glutaric acid-d4 (purity: 99%) were obtained from Medical Isotopes (Pelham, NH, USA). Ethylmalonic acid-methyl-d3 (purity: 98%) was purchased from Cambridge Isotopes Laboratories (Tewksbury, MA, USA). Optima-LC/MS grade water, acetonitrile, 2-propanol, and methanol were obtained from Fisher Scientific (Pittsburgh, PA, USA). Blank human plasma (collected in K2-EDTA tubes) and blank human urine were purchased from BIOIVT (Westbury, NY, USA) LC-MS grade water and LC-MS grade acetonitrile were purchased from ThermoFisher Scientific (Waltham, MA, USA). UriSub was purchased from CST Technology (Great Neck, NY, USA). The n-butanol with 3 M HCl was obtained from Regis Technologies (Morton Grove, IL, USA)

### LC-MS/MS Analysis

MRM analyses were performed on a 6500 QTRAP Mass Spectrometer (MS) (Sciex, Framingham, MA) equipped with an electrospray source, a 1290 Infinity UPLC system (Agilent Technologies, Santa Clara, CA) and, Luna Omega, 1.6 μm PS C18 100A (100×2.1 mm) column (Phenomenex, Torrance, CA, USA). Liquid chromatography was carried out at a flow rate of 400 μL/min, and the sample injection volume was 10 μL. The column was maintained at a temperature of 60° C. Mobile phase A consisted of 0.1% formic acid (FA) in water and mobile phase B consisted of 0.1% FA in acetonitrile. The gradient with respect to % B was as follows: 0 to 2 min, 20%; 2 to 4 min, 20% to 50%; 4 to 13 min, 50%; 13 to 15 min, 95%; 15.1 to 20 min, 20%. The instrument parameters for 6500 QTRAP MS were as follows: Ion spray voltage of 5500 V, curtain gas of 30 psi, collision gas set to “High”, interface heater temperature of 550° C., nebulizer gas (GS1) of 40 psi and ion source gas (GS2) of 40 psi and unit resolution for both Q1 and Q3 quadrupoles. The bis-ester form of MSA, EMA and GA were quantified in this study. The MRM parameters for derivatized MSA, EMA, and GA with their corresponding I.S. were summarized in FIG. 1B. The performance of derivatization using acidified n-butanol was examined following different incubation times for 15, 30, 45, and 60 min at 60° C. At these incubation times, similar intensities of these C5-isomers in MS were demonstrated at each level of lowest and highest concentration of calibrators (data not shown). The incubation for 30 min at 60° C. was chosen for the following sample preparation with derivatization using acidified n-butanol.

### Calibrators and Quality Control (OC) Sample Preparation

Calibrator concentration range of 5.00/10.0/20.0 to 400/400/400 ng/mL in SigMatrix for the plasma assay and 100/200/100 to 5000/10000/5000 ng/mL in UriSub for the urine assay were prepared. Calibrator concentrations for MSA, EMA, and GA were listed in Table 1. For the preparation of Quality Control (QC) samples, human plasma and urine from both male and female lots were screened to determine the endogenous levels of MSA, EMA and GA. The Low-pool and Mid-pool of human plasma and urine were prepared based on their endogenous basal levels. The Low-pools of human plasma and urine were prepared for low QC samples (LQC) by combining a minimum of 2 male and 2 female plasma lots in plasma assay, as well as a minimum of 3 male and 3 female urine lots in urine assay. Dilution QC samples was prepared in Low-pools of human urine only for the urine assay, from a 5-fold dilution of 10000/20000/10000 ng/mL for MSA/EMA/GA. The Mid-pools of human plasma and urine were prepared for medium QC sample (MQC) combining a minimum of 5 male, and 5 female plasma lots. High QC samples (HQC), were prepared by combining a minimum of 6 male and 6 female urine lots. The lowest limit of quantification QC samples (LLOQ-QC) were prepared in SigMatrix and UriSub in both assays. A Fit-for-purpose approach was used and QC ranges were generated by using data obtained during validation. Six replicates of each QC sample were extracted in each batch and six batches were analyzed during validation. A minimum of 36 data points collected at each QC level may be used for generating the QC concentration ranges. At each QC level, the range was generated by using the following equation: Inter-assay QC mean±[(Z)×(standard deviation)] (Z is defined for each QC from minimum 6 batches in validation, evaluated based on the reference of the assay capability index (Cp) [23].

### Sample Preparation

All solutions and reagents were brought to room temperature (RT) before initiating the extraction process. standards (STD), QC samples, surrogate matrix, and unknown human plasma/urine samples were thawed at RT. Acetonitrile (400 μL and 200 μL for the plasma and urine assay, respectively) was added into tubes along with working-IS solution (20 μL), except for the double blank. STD's, QC's and unknown samples (200 μL and 100 μL for plasma and urine assay, respectively) were added into each tube and vortexed immediately for 2 seconds. All samples were vortexed for 5 min by a multi-tube vortex mixer (VWR International LLC, Radnor, PA). Samples were centrifuged at 4° C. for 20 min at a speed of 17,000×g. Each sample (400 μL for the plasma assay and 200 μL for the urine assay, respectively) was transferred into separate, new, amber microcentrifuge tubes. Samples were centrifuged at 4° C. for approximately 10 seconds at 17,000×g. Samples were then dried using a Turbovap (Caliper Life Sciences, Inc, Hopkinton, MA, USA) under a gentle stream of nitrogen gas at 37° C. 3 M HCl in n-butanol (50 μL) was added into each tube and all samples were vortexed. Samples were incubated for 30 min, at 60° C. and shaken at 500 rpm. All samples were centrifuged at 4° C. for 10 seconds at 17,000×g, and then dried using a Turbovap under a gentle stream of nitrogen gas at 37° C. All samples were reconstituted by adding 150 μL of reconstitution solution (50:50:0.1 MeOH:H2O:FA), and vortexed for 10 seconds. Samples were analyzed by LC-MS/MS.

### Assay Validation

The following parameters were assessed during assay validation:

Calibration curve linearity. The linearity of six independent calibration curves for MSA, GA, and EMA were assessed in the plasma and urine assays.

Intra and inter-batch precision. The intra- and inter-batch precision was evaluated by analyzing the LLOQ-QC, LQC, MQC and HQC with 6 replicates for each on different days.

Matrix effect assay. Human K2-EDTA plasma and urine were spiked with the LLOQ-QC and HQC concentrations and assessed against calibration standards. Blank samples of plasma and urine as a zero standard from a minimum of 8 different lots were extracted to determine the basal concentrations of MSA, EMA, and GA. These basal values were spiked by LLOQ-QC and HQC concentrations to obtain the actual nominal concentrations and compared with the measured LLOQ-QC and HQC concentrations.

Short-term stability (STS) and long-term stability (LTS) of calibrator in surrogate matrixes. Standard solutions for MSA/EMA/GA were examined at the lowest concentrations with 5.00/10.0/20.0 ng/mL in SigMatrix and 100/200/100 ng/mL in UriSub. The highest concentration was 400/400/400 ng/mL in SigMatrix and 5000/10000/5000 in UriSub in both the plasma and urine assay, respectively. In STS, aliquots of calibrator were assessed by leaving both lowest and highest calibrators up to 24 hrs at RT. These exposed samples were compared against unexposed samples. In LTS, aliquoted calibrators were stored at −80° C. and evaluated by comparing the aliquots stored at −80° C. against freshly prepared standards of the same concentration.

STS, LTS, and freeze-thaw stability (FTS) of human plasma and urine samples. LQC and HQC samples were used for assessment of STS at RT for 4 hrs in the plasma assay, and 24 hrs in the urine assay, for evaluation of LTS at −80° C., and for assessment of FTS up to four cycles at −80° C. and RT. In FTS, each freeze cycle was for a minimum of 24 hrs. Both QC samples in triplicate were compared against the QC range generated during the validation. Two thirds of the sample concentrations must fall within the established QC range generated during validation.

Re-injection reproducibility in the autosampler. Six replicates of each LLOQ-QC, LQC, MQC and HQC samples were injected with a set of calibration standards. The batch stored at 4° C. in the autosampler was re-injected, for 2, 4, and 7 days in the plasma assay, and for 7, and 11 days in the urine assay.

Interference assessment. Potential interferences were evaluated by individually spiking LQC and HQC samples with human hemolysate (500 mg/dL), unconjugated bilirubin (30 mg/dL), and triglycerides (1000 mg/dL), compared to the unspiked QCs in the plasma assay. For the urine assay, the LQC and HQC in UriSub spiked with human serum albumin (HSA) (final concentration 0.25 mg/mL and 1.00 mg/mL), and with pH additives containing HCl or NaOH (final pH 3.0±1 and pH 10.0±1) were analyzed, compared to the unspiked QCs.

System suitability, drift, and carryover. System suitability was assessed by calculating the precision (CV,%) of the calculated concentration from 4 replicates of the system suitability standard (SSS) at the beginning of each batch, which were required to be ≤10%. The drift for SSS was assessed by calculating the bias (%) of the mean concentrations from 4 replicates at the beginning and 2 replicates at the end of each batch. The drift (bias,%) between the beginning and the end of the batch were required to be ≤20%. In the carryover evaluation, a reconstitution solution was injected after the upper limited of quantification standard (ULOQ-std). The carryover was calculated by evaluating the peak area of analytes for MSA, EMA, and GA in reconstitution solution, which were required to be <20% of the peak area of analytes in LLOQ standards.

### Data Analysis

The retention time and peak area for the analytes of interest were determined using the quantitation function on the Analyst® software (AB Sciex Version 1.6.2) and MultiQuant (Version 3.0.1). Calibration curves for MSA, EMA, and GA were constructed by plotting the peak area ratio (y) of analyte to internal standard versus the concentration of the analyte (x). The calibration curve was fitted initially to a non-weighted linear model of the form y=ax+b. The 1/x² weighting factor was optimal for evaluating the curves of MSA, EMA and GA found in the validation. The analyte concentration in each unknown sample and standard was determined by back-calculation using the following relationship from each fitted calibration curve: x=(y−b)/a. Precision is the degree of agreement among individual measurements produced by the assay system under a defined set of conditions. Intra- and inter-assay precision were expressed in terms of the coefficient of variation expressed as percent (CV,%), based on 6 replicates and a minimum of 6 batches respectively, calculated as follows:

$$( C V , \% ) = \frac{Standard Deviation}{M e a n} \times 100$$

The differential changes in assay were determined using (bias,%) calculation:

$$( Bias , \% ) = \frac{[ Actual Conc . - Nominal Conc . ]}{[ Nominal Conc . ]} \times 100$$

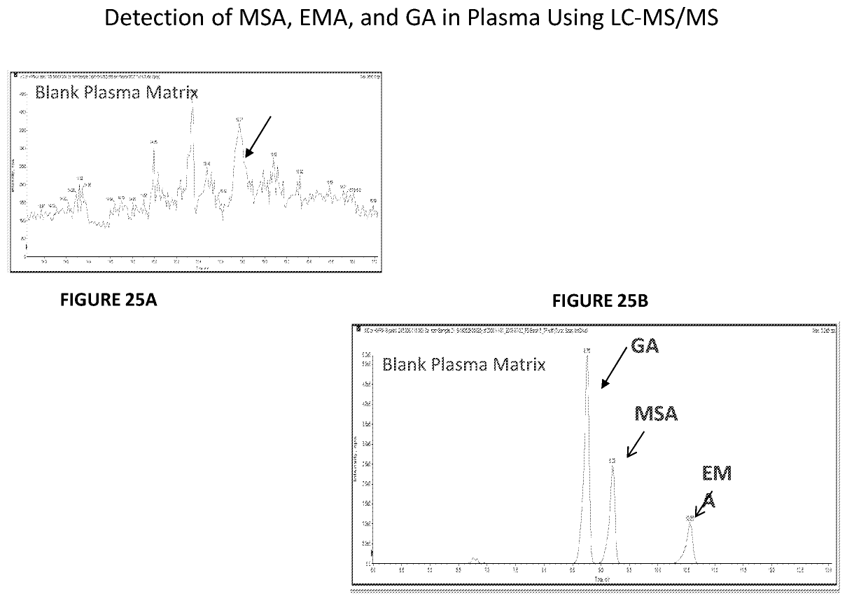

FIGS. 25A and 25B are LC-MS/MS spectra obtained when separation was performed using a previous method (FIG. 25A) and using the LC-MS/MS assay described herein (FIG. 25B). Poor isomer separation and identification, as well as poor signal intensity were obtained using the prior metabolomics-based method (FIG. 25A). In contrast, good separation of the three isomers were obtained using the multiplex LC-MS/MS assay described herein, as illustrated in the spectrum shown in FIG. 25B.

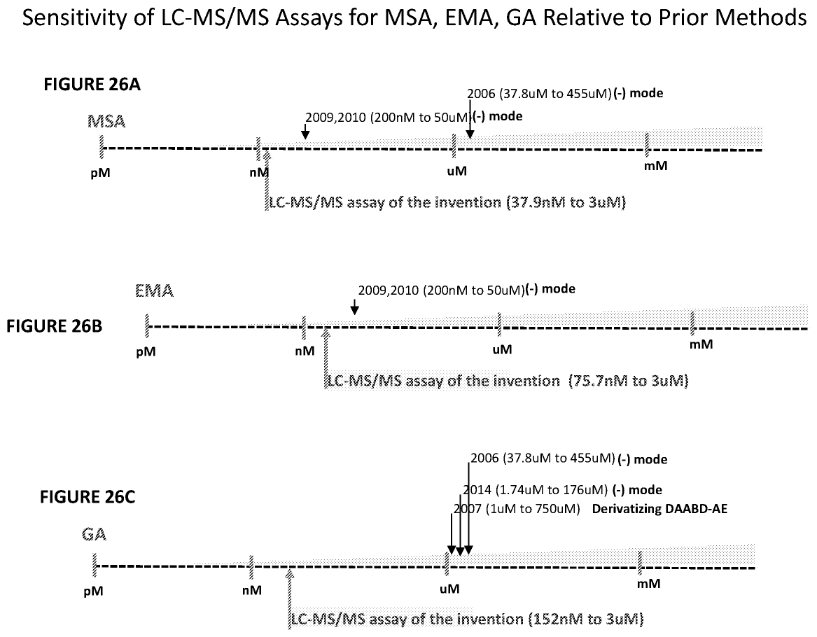

Improved chromatography and signal intensity were achieved in the LC-MS/MS method for MSA, EMA and GA compared to a previously known method. FIGS. 26A, 26B and 26C indicate the level of sensitivity of the LC-MS/MS assay method compared to the sensitivity achieved using a prior assay method for each isomer. Thus, the LC-MS/MS method described herein was a sensitive, robust and reliable assay for detection of MSA, EMA and GA.

Detection and Quantitation of NAP using LC-MS/MS. To quantify NAP in human K2-EDTA plasma, the quantification method using LC-MS/MS was developed. Due to low circulating levels of NAP, the sensitivity of quantification was improved using derivatization of isobutyl chloroformate in the sample preparation. The validation performance of the NAP assay is summarized in Table 8-15, including linearity, precision, matrix effect, system suitability, short-term stability, long-term stability, and reproducibility in the autosampler. Fit-for-purpose method validation results demonstrated quantitative ranges for NAP from 1 ng/mL to 85 ng/mL in plasma analysis (Table 10). The results of validation assessed by QCs met acceptance criteria (Table 9-15).

TABLE 8
Summary Table of MRM Parameters

| | Q1 Mass | Q3 Mass | Dwell Time | DP | EP | CE | CXP |
|---|---|---|---|---|---|---|---|
| Compound | (amu) | (amu) | (msec) | volts | volts | volts | volts |
| N-Acetylputrescine | 231.0 | 115.0 | 300 | 60 | 10 | 20 | 25 |
| (NAP1) | | | | | | | |
| N-Acetylputrescine | 231.0 | 157.0 | 300 | 60 | 10 | 20 | 25 |
| (NAP2) | | | | | | | |

TABLE 9
Validation Summary for NAP in Plasma Assay

| | | | Assay |
|---|---|---|---|
| Category | Study | | Performance |
| Standard and QCs | Linearity | LLOQ std. (% Bias) | <2% |
| performance | | other std. (% Bias) | <5% |
| | Precision | intra-assay precision (% CV) | <10% |
| | | inter-assay precision (% CV) | <26% |
| System Performance | Matrix Effect | LLOQ-QC (ME %, % Bias) | 90%; −10% |
| | | HQC (ME %, % Bias) | 76%; −24% |
| | Interference | Hemolysate, Bilirubin & | >15% ** |
| | | Lipoproteins; LQC & HQC | |
| | | (% Bias) | |
| | System Suitability | % CV | <3% |
| | System Suitability Drift | % Drift | <9% |
| | Carryover | % Carryover | 0% |
| Stability | Standard Solutions | STS (RT, 24 h) | −4% |
| | in Surrogate Matrix | LLOQ std. & substock* (% Bias) | |
| | | LTS (−80 C., 88 day) | 11%; −8% |
| | | LLOQ std. & ULOQ std | |
| | | (% Bias) | |
| | Human Plasma | STS (RT, 24 h) | 7%; 1% |
| | | LQC & HQC (% CV) | |
| | | LTS (−80 C., 84 day) | 2%; 3% |
| | | LQC & HQC (% CV) | |
| | | FTS | 7%; 11% |
| | | LQC & HQC (% CV) | |
| Reproducibility | Re-injection at 4° C. in | Reproducibility (4 C., 8 day) | 3%; 14% |
| | autosampler | LQC & HQC (% CV) | |

TABLE 10
Calibration Curve Ranges and Linearity for NAP in Plasma Assays.

| NAP | Plasma assay (n = 4) | | |
|---|---|---|---|
| Spiked Nominal | Mean Measured | | Average % |
| Concentration (ng/ml) | Concentration (ng/ml) | % CV | Bias |
| 1.0 | 1.0 | 3.5 | 1.5 |
| 2.0 | 1.8 | 5.2 | 4.1 |
| 5.0 | 5.0 | 6.6 | 2.4 |
| 10.0 | 10.1 | 3.1 | 2.0 |
| 25.0 | 23.9 | 3.9 | 2.0 |
| 50.0 | 52.4 | 2.7 | 2.6 |
| 68.0 | 70.8 | 6.0 | 1.4 |
| 85.0 | 83.4 | 4.5 | 2.8 |

Note:
The linearity for NAP (r² = 0.99745, n = 4)

TABLE 11
The Intra- and Inter-Assay Precision (% CV) in Average for Six Batches

| | Intra-assay | Inter-assay |
|---|---|---|
| QC | (% CV) | (% CV) |
| LLOQ QC | 9.8 | 14.7 |
| LQC | 5.2 | 8.8 |
| MQC | 4.8 | 7.4 |
| HQC | 9.5 | 25.6 |

TABLE 12
Matrix Effect Summary for NAP in Plasma Assay
spiked with NAP in plasma (n = 9)

| | spiking (ng/mL) | ME % | % Bias |
|---|---|---|---|
| LLOQ QC | 1.0 | 89.6 | −10.4 |
| HQC | 64 | 75.9 | −24.1 |

TABLE 13
Short-Term Stability, and Long-Term Stability for NAP
Spiked in 2.5% BSA Standard Solution

| | | Concentration | Mean | | |
|---|---|---|---|---|---|
| Stability | | (ng/mL) | (peak area) | CV % | Bias % |
| STS | L1 | 1.0 | 2.8E+04 | 1.3 | −3.6 |
| (24 hrs, 5 C.) | substock | 2000 | 1.6E+06 | 1.4 | −6.7 |
| LTS | L1 | 1.0 | 4.3E+04 | 6.1 | 10.6 |
| (88 days, −80 C.) | L8 | 85.0 | 3.0E+06 | 8.2 | −8.1 |

TABLE 14
Short-Term Stability, and Long-Term Stability for NAP Spiked in Human Plasma

| | | | | | | | | | Re-injection | |
|---|---|---|---|---|---|---|---|---|---|---|
| | | | Short-term | | Long-term | | Freeze-thaw | | stability in | |
| | | | stability | | stability | | stability | | autosampler | |
| | Expected | Acceptable | (24 h, RT) | | (84 days, −80° C.) | | (4x) | | (8 days) | |
| | Value | Ranges | Mean | | Mean | | Mean | | Mean | |
| | (ng/mL) | (ng/mL) | (ng/mL) | CV % | (ng/mL) | CV % | (ng/mL) | CV % | (ng/mL) | CV % |
| LQC | 3.2 | LQC: | 3.1 | 6.9 | 3.2 | 2.2 | 3.1 | 6.8 | 3.2 | 3.5 |
| | | 2.33-4.03 | | | | | | | | |
| HQC | 54.2 | HQC: | 41.0 | 1.2 | 63.4 | 2.8 | 42.4 | 11.0 | 46.5 | 13.8 |
| | | 27.8-80.7 | | | | | | | | |

TABLE 15
Interference in Plasma Assay

| | Unspiked | | | | | | | | | |
|---|---|---|---|---|---|---|---|---|---|---|
| | interference | Hemolysate | | | Bilirubin | | | Lipoproteins | | |
| | Mean | Mean | | Bias | Mean | | Bias | Mean | | |
| | (ng/mL) | (ng/mL) | CV % | (%) | (ng/mL) | CV % | (%) | (ng/mL) | CV % | % Bias |
| LQC | 3.7 | 3.1 | 3.3 | −16.1 | 3.0 | 3.2 | −18.4 | 13.4 | 3.2 | 264.0 |
| HQC | 49 | 47 | 2.1 | −3.8 | 45 | 1.3 | −7.5 | 57 | 1.0 | 16.5 |

Note:
In plasma assay, potential interferences were evaluated by spiking HQC and LQC samples with hemoglobin (500 mg/dL), unconjugated bilirubin (30 mg/dL), and triglycerides (1000 mg/dL).

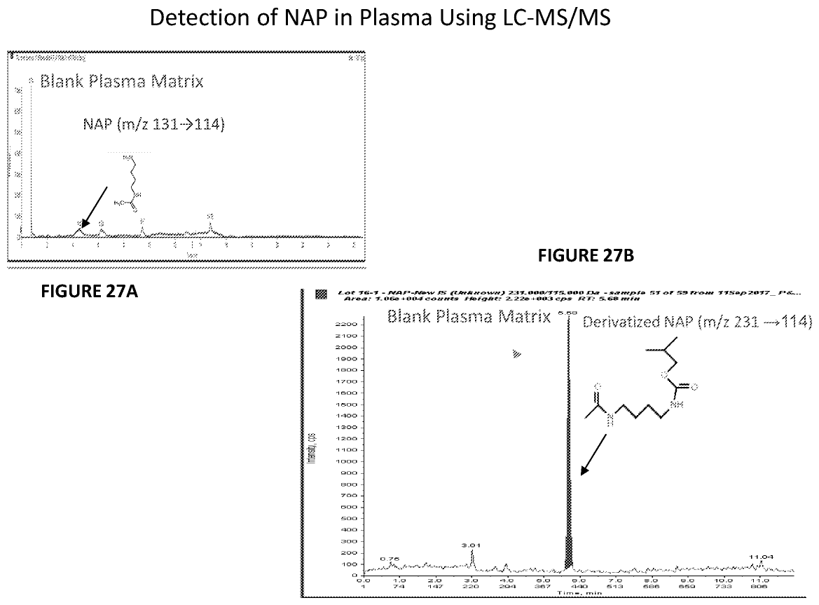

FIGS. 27A and 27B are LC-MS/MS spectra obtained when separation was performed using a previous method for metabolomics (FIG. 27A) and using the LC-MS/MS assay described herein (FIG. 27B). Poor signal intensity was obtained using the prior metabolomics-based method (FIG. 27A), and thus, quantitation of NAP in plasma was challenging. In contrast, the assay method of the invention provided improved signal intensity, cleaner samples, and improved chromatography (FIG. 27B). The LC-MS/MS method described herein was sensitive, robust and reliable for detection and quantitation of NAP.

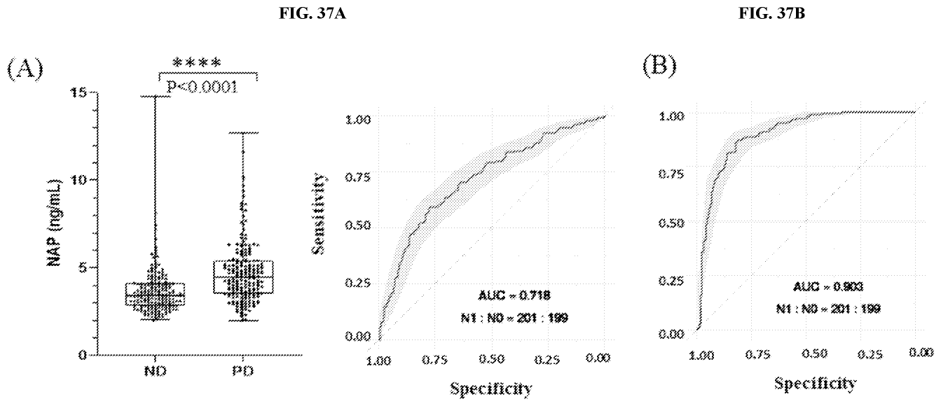

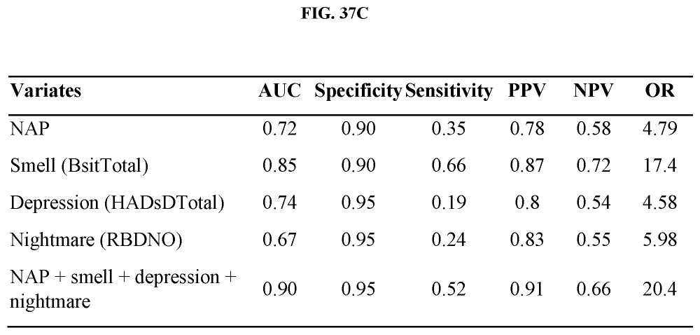

Utilizing our CLIA validated quantification method, the K₂-EDTA plasma samples from a total of 400 participants, including 199 non-disease and 201 PD cohort, were analyzed for NAP quantitation. When comparing PD in male and female patients, there were no statistical differences of plasma levels of NAP in gender that was observed (one-way ANOVA, p=0.6492) (Data not shown). The significant difference of plasma NAP between ND and PD was detected (t-test, p<0.0001). The mean concentrations of NAP in the plasma for ND and in the PD cohorts were 3.70 ng/mL and 4.74 ng/mL, respectively (FIGS. 37A-37C).

NCAM Detection Method. Plasma NCAM was detected and quantitated using the methods described in Guven et al., 2021, Journal of Pharmaceutical and Biomedical Analysis 197: 113981, which is incorporated by reference herein in its entirety.

### NCAM-1 Immunoassay Development

Human NCAM-1 DuoSet (R&D Systems, Cat #DY2408) that contains monoclonal mouse anti-human NCAM-1 capture antibody (R&D Systems. Cat #842183) and biotinylated polyclonal goat anti-human NCAM-1 detection antibody (R&D Systems, Cat #842184) was used to develop an NCAM-1 assay. Sulfo-Tag labeled Streptavidin was chosen as the detection reagent (MSD, Cat #R32AD-1).

### Optimization of Antibody Pair

A checkerboard titration assay was employed. Capture and detection antibody concentrations were optimized by titrating each antibody on multi-array 96-well standard plates (MSD, Cat #L15XA-3). 2-fold dilution series was used for both capture and detection antibody. The concentrations of capture antibody and detection antibody were varied with respect to each other. Capture antibody concentrations were prepared in phosphate buffered saline (PBS) (Fisher Scientific, Cat #10010031) at varying concentrations from 2 to 0.25 μg/mL. 96-well plates were coated with 30 μL of capture antibody concentrations overnight at 4° C. The wells of the plate were subsequently blocked with 150 μL of StartingBlock T20 blocking buffer (Fisher Scientific, Cat #37539) for 1 h at room temperature (RT, 18-24° C.) at 750 rpm. The wells of the plate were washed three times with PBS/Tween (PBST, Sigma Aldrich. Cat #08057-100TAB-F) using an automated plate washer (Biotek EL406 Plate Washer) to remove the excess reagents. Twenty-five μL purified NCAM-1 protein (R&D Systems, custom protein) at a concentration of 3,000 pg/mL in Hispec assay diluent (Bio-Rad, Cat #BUF049C) was added and plates were incubated for 2 h at RT (18-24° C.) at 750 rpm. Twenty-five μL of Hispec assay diluent alone was added for blank. Following another wash step (six times in this step), 30 μL/well of the detection antibody prepared in blocking buffer at varying concentrations from 4 to 1 μg/mL, was added and incubated at RT and 750 rpm for 1 h. The plate was washed six times and 30 μL of sulfo-tag labeled streptavidin was added to each well. Plates were incubated for 30 minutes at RT and 750 rpm. Subsequently, 150 μL of Read Buffer (MSD, Cat #R92TC-1) was added and the plates were read immediately on the MSD SECTOR Imager (MSD, QuickPlex SQ 120 Reader). For each antibody pair, the signal-to-noise ratio was calculated by dividing the ECL value of the signal when the purified NCAM-1 is present, by the ECL value of the signal from blank. The dilutions which returned the strongest signal-to-noise ratio were selected.

### Optimization of Blocking Conditions

To determine which blocking buffer performed best during the blocking step, different commercially available blocking buffers were tested (StartingBlock (Fisher Scientific, Cat #37539), SuperBlock (Thermo Fisher Scientific, Cat #37515) and Blocker A solution (MSD, Cat #R93BA-4)). 96-well plates were coated with 30 μL of capture antibody at a concentration of 0.25 μg/mL in PBS overnight at 4° C. The wells of the plate were subsequently blocked with 150 μL of one of the three blocking buffers for 1 h at RT (18-24° C.) at 750 rpm. The wells of the plate were washed three times with PBST using an automated plate washer. Twenty-five L purified NCAM-1 protein at a concentration of 3,000 μg/mL in Hispec assay diluent was added and plates were incubated for 2 h at RT (18-24° C.) at 750 rpm. Twenty-five μL of Hispec assay diluent alone was added for blank. Following another wash step (six times in this step), 30 μL/well of the detection antibody at a concentration of 1 μg/mL in blocking buffer, was added and incubated at RT and 750 rpm for 1 h. The plate was washed six times and 30 μL of sulfo-tag labeled streptavidin was added to each well. Plates were incubated for 30 minutes at RT and 750 rpm. Subsequently, 150 μL of Read Buffer was added and the plates were read immediately on the MSD SECTOR Imager. For each blocking buffer, the signal-to-noise ratio was calculated by dividing the ECL value of the signal when the purified NCAM-1 is present, by the ECL value of the signal from blank. The blocking buffer which yielded the highest signal-to-noise ratio was selected.

### Human Anti-Mouse Antibody (HAMA) Interference

To minimize the potential effect of HAMA, an excess of non-relevant mouse IgG antibody (Jackson ImmunoResearch, Cat #015-000-003) was added at a concentration of 10 μg/mL. For this purpose, 96-well plates were coated with 30 μL of capture antibody at a concentration of 0.25 μg/mL in PBS overnight at 4° C. The wells of the plate were subsequently blocked with 150 μL of StartingBlock for 1 h at RT (18-24° C.) at 750 rpm. The wells of the plate were washed three times with PBST using an automated plate washer. Twenty-five μL purified NCAM-1 protein at concentrations 300, 1500 and 3000 pg/mL and three 2-fold human plasma dilutions, starting at 1:100 in Hispec assay diluent were added and plates were incubated for 2 h at RT (18-24° C.) at 750 rpm. Twenty-five μL of Hispec assay diluent alone was added for blank. Following another wash step (six times in this step), 30 μL/well of the detection antibody at a concentration of 1 μg/mL in blocking buffer, was added with and without mouse IgG antibody and incubated at RT and 750 rpm for 1 h. The remaining steps of the assay were performed as described above. The signal-to-noise ratio was calculated for each well. The effect of mouse IgG on HAMA susceptibility was determined by comparing signal-to-noise from experiments run both with and without mouse IgG inclusion.

### Calibration Curve

A dose response curve was generated for the NCAM-1 sandwich assay by spiking two-fold serial dilution of the 200,000 pg/mL purified NCAM-1 stock in Hispec assay diluent. The assay was run as described above using detection antibody together with mouse IgG. NCAM-1 nominal concentration (%) is a terminology that has been used throughout this manuscript and relates the calculated amount to the known added amount of the calibrator. NCAM-1 nominal concentration (%) was assessed in the range of 312.5-200,000 pg/mL. Concentrations, spike recoveries and standard deviations for each calibration point were calculated by regression analysis using five-parameter logistic curve-fitting with Discovery Workbench 4.0 software (MSD).

### Parallelism and Matrix Effects

To evaluate parallelism, eight human individual plasma (BioIVT) with six 2-fold dilutions between 1:50 and 1:1600 were tested. Dilutions were prepared using Hispec assay diluent. The minimum dilution achieving parallelism was chosen as the minimum required dilution (MRD). To assess matrix effects, human individual plasma was spiked with purified NCAM-1 at concentrations of 10,000 pg/mL (High spike) and 5,000 pg/mL (Medium spike) and 2,500 pg/mL (Low spike). Plasma, spiked Hispec assay diluent and spiked plasma were each assayed in duplicate on the same plate. Nominal concentration (%) was calculated using the equation shown below.

$$Nominal Concentration ( \% ) = \frac{[ Measured Conc . ]}{[ Spiked Conc . + Endogenous Conc . ]} \times 100$$

### Sensitivity

Ten 2-fold dilutions of purified NCAM-1 were prepared beginning with 50,000 pg/mL in Hispec assay diluent and assayed in duplicate, along with 24 replicates of the zero-concentration Hispec assay diluent blank. A similar procedure was followed for pooled human plasma which was subjected to eleven 2-fold dilutions from 1:10 to 1:16000. Sigmoidal, 5PL, interpolation approach was used to evaluate assay sensitivity. The minimum detectable concentration (MDC) was determined as the concentration at which a response greater than the blank with an acceptable nominal concentration is achieved. The reliable detection limit (RDL) was determined as the lowest concentration of analyte that produces a response significantly greater than the blank.

### Procedure of the MSD Based Sandwich Immunoassay for Quantification of NCAM-1 in Plasma

A schematic of the assay procedure can be found in FIG. 1. Multi-Array 96-Well Standard Plates were coated with 30 μL mouse anti-human NCAM-1 capture antibody at a 0.25 μg/mL concentration, in PBS. Plates were covered with adhesive sealing film and incubated overnight (>16 h) at 4° C. The next day, the capture antibody solution was removed by emptying the plate and blotting it on paper towels. 150 μL of StartingBlock T20 blocking buffer was added to each well and the plates were sealed and blocked for 1 hour at RT (18-24° C.) and 750 rpm. A calibration curve was generated by ×2 serial dilution in Hispec assay diluent. Working concentrations of 20,000, 10,000, 5,000, 2,500, 1,250, 625, 312.5 and 0 pg/mL were used in the assay. Plates were washed three times with PBST using an automated plate washer to remove the excess reagents. Human plasma was diluted 1:200 in Hispec assay diluent. Twenty-five μL of each standard and sample were added to the plate in duplicate. Hispec assay diluent alone was used as a blank. Plates were incubated for 2 hours at RT (18-24° C.) at 750 rpm.

Plates were then emptied and washed six times with PBST wash using an automated plate washer to remove the excess reagents. A mixture of biotinylated goat anti-human NCAM-1 detection antibody at 1 μg/mL working concentration and normal mouse IgG at 10 μg/mL working concentration in Hispec assay diluent was prepared. 30 μL of this mixture was added to each well. Plates were sealed and incubated for 1 hour at RT (18-24° C.) at 750 rpm.

After emptying the detection antibody solution and washing the plate six times, Sulfo-tagged Streptavidin was diluted in Hispec assay diluent to a final concentration of 1 μg/mL. 30 μL of this solution was added to each well. Plates were sealed and incubated for 30 minutes at RT (18-24° C.) at 750 rpm.

Subsequently, MSD Read Buffer was added, and the plates were read immediately on the MSD SECTOR Imager.

### Assay Validation

### Precision

Twenty human plasma samples were screened to identify a low, medium and high concentration of NCAM-1. The identified plasma was then utilized for precision studies and later as the low-quality control (LQC), medium quality control (MQC) and high quality control (HQC) samples. The samples were aliquoted (50 μL/tube) and stored in screw top cryotubes at −80° C. until measurement. On the day of experiment, samples were diluted 1:200 in Hispec assay diluent and tested in triplicate with one run per day for six days. Intra- and Inter-assay precision was determined and expressed as the coefficient of variation (CV %) using the equation below:

$$C V \% = \frac{Standard Deviation}{Average Measured Concentration} \times 100$$

### Accuracy

Accuracy was determined as the percentage of the observed concentration of known amount of standard spiked into plasma matrix and expressed as percent nominal concentrations. Known concentrations of purified NCAM-1 were spiked into pooled healthy individual plasma and calculating the interpolated results back to the concentrations. These experiments were performed on six separate days with six separate plates. We have calculated the accuracy for three different concentrations of NCAM-1 that fall within the linear range of the assay.

### Selectivity

The selectivity study was performed by testing ten human individual plasma samples spiked with purified NCAM-1. 10 μL of 20 μg/mL purified NCAM-1 was added to 90 μL of human plasma and Hispec diluent assay for a final spike concentration of 2 μg/mL. 10 μl of and 4 μg/mL purified NCAM-1 was added to 90 μL of human plasma and Hispec diluent assay for a final spike concentration of 400,000 pg/mL. (NCAM-1 spiking volume is 10% of the final test volume). Following that, plasma, spiked Hispec diluent assay diluent and spiked plasma were diluted 200 times resulting in final NCAM-1 spike concentrations at 10,000 pg/mL (high spike) and 2,000 pg/mL (low spike). Each sample then assayed in duplicate on the same plate. Nominal concentrations (%) were calculated by dividing the measured NCAM-1 concentration by the assay by the theoretical concentration of the sample (spike concentration+NCAM-1 concentration in the unspiked sample in pg/mL). The assay passed the analysis criteria when the nominal concentrations were within 80% and 120% [22].

### Short-Term Stability

In the short-term stability test, three levels of QC samples were thawed from the −80° C. freezer and incubated at 4° C. or RT (18-24° C.) for 2, 4 or 24 h. The samples were then assayed and compared with samples freshly thawed from the −80° C. freezer and assayed along with the samples incubated at different conditions on the day of the assay.

### Freeze-Thaw Stability of Samples

Freeze-thaw stability was determined over three freeze-thaw cycles. QC samples from three concentration levels (high, medium and low) were thawed between 1-3 times by removal from the −80° C. freezer and thawing them on ice for ≤1 hour. Samples were then returned to the freezer and stored as before. QCs were diluted 1:200 and assayed on the same plate, with 3 replicates per sample. Any significant changes in assay response were determined using Bias % calculation using the equation shown below:

$$Bias \% = \frac{[ Actual Conc . - Nominal Conc . ]}{[ Nominal Conc . ]} \times 100$$

### Interference

Assurance™ interference test kit (Sun Diagnostics, New Gloucester, ME) was used for bilirubin, lipid, and hemoglobin spiking. Biotin was purchased from Fisher Scientific (Cat #BP232-1). Interfering substances were spiked in 10% of the final volume of pooled plasma at seven serially diluted concentrations and samples were assayed normally. Bias % was calculated for each sample to determine at which concentration the interference yielded a significant change in assay response.

ITIH4 Detection Method. Plasma ITIH4 was detected and quantitated using the following method.

TABLE 16
Materials and Methods

| | | Catalog | | Storage |
|---|---|---|---|---|
| Materials | Vendor | Number | Lot Number | Conditions |
| Uncoated MSD multi- | Meso Scale Diagnostics | L15XA-3 | — | RT |
| Array 96-well standard | | | | |
| plates | | | | |
| Reservoir | Thermo Fisher | NC0382499 | — | RT |
| | Scientific | | | |
| PBS | Thermo Fisher | 10010031 | — | RT |
| | Scientific | | | |
| Phosphate buffered | Sigma | 08057- | — | RT |
| saline/Tween tablets | | 100TAB-F | | |
| StartingBlock T20 | Thermo Fisher | 37539 | — | 4° C. |
| Buffer | Scientific | | | |
| StartingBlock (PBS) | Thermo Fisher | 37578 | — | 4° C. |
| Assay Diluent | Scientific | | | |
| Biotinylated anti- | R&D Systems | Custom-AB | CINS011711A | 4° C. |
| human ITIH-4 | | | | |
| Sulfo-Tag Streptavidin | Meso Scale Diagnostics | R32AD-1 | W00165975 | 4° C. |
| ECL reading buffer | Meso Scale Diagnostics | R92TC-1 | Y0140344 | RT |

Sandwich Assay: Capture antibody is diluted to the working concentration (1 μg/ml). MSD Multi-array 96-well Standard plates are coated with 30 μl of the diluted capture antibody solution. Plate is sealed and incubated at 4° C. overnight. The antibody solution is discarded and plate is incubated with 150 μl of StartingBlock with shaking at room temperature for 1 hour. Plate is washed 3× with PBST. Standard, QC and sample dilutions are made in Startingblock (PBS) assay diluent buffer (See assay SOP for sample preparation guideline) and 25 μl of prepared sample is added to the wells and plate is incubated at RT with shaking (750 rpm) for 2 hours. Plates are washed 6× with PBST. Detection antibody (working concentration: 1 μg/mL) in StartingBlock is prepared. 30 μl of mixture is added per well. Plate is incubated for 1 hour with shaking (750 rpm) at RT. Antibody solution is discarded and plate is washed 6× with PBST. Sulfo-tag streptavidin is diluted to the working concentration (1 μg/ml) and 30 μl is added per well. Plate is incubated for 30 min with shaking (750 rpm) at RT. Plate is washed 9× with PBST and 150 μl of 2× Read buffer is added into each well. Plates are read with QUICKPLEX immediately.

### Calibration Curve

In the current qualification experiments, seven non-zero Calibration Standards were prepared in StartingBlock (PBS) assay diluent buffer (pg/ml): 150000, 50000, 16667, 5556, 1852, 617, 206. MSD's software (Discovery workbench 4.0.12.1) was used for calculations with curve fitting method of Four Parameter Logistic (4PL) nonlinear regression model and 1/Y² weighting.

The recovery of an analyte in an assay is the detector response obtained from an amount of the analyte added to and extracted from the biological matrix. The correlation of determinations (R²) of the standard curve must be ≥0.90 for the signal versus concentration. Using back-calculated concentrations, at least 75% of non-zero standards (6 out of 7 standard points) should meet the following criteria:
1. Recovery of an analyte is the measured concentration relative to the known amount added to the matrix. The standard calibrator concentrations should be within 25% of the nominal concentration at LLOQ and within 20% of the nominal concentration at all other concentrations. The standard calibrator points show recovery between 75-125% for LLOQ and 80-120% for all other concentrations meet the acceptance criteria. % Recovery is calculated as follows:

$$\% Recovery = ( Measured Concentration \div Spiked Concentration ) \times 100$$

2. Precision—is the degree of agreement among individual measurements produced by the assay system under a defined set of conditions. Precision will be expressed in terms of the coefficient of variation expressed as percent (% CV) calculated as follows:

$$\% CV ( Standard deviation ( SD ) \div Average Measured concentration ) \times 100$$

1.1.1 Precision Study with QC Materials:

According to US FDA, at least three concentrations of QCs should be incorporated into each run as follows: one within three times the LLOQ (Low QC), one in the midrange (middle QC), and one approaching the high end (high QC) of the range of the expected study sample concentrations. In addition, QCs should be made in the same matrix of the study sample. The calibration range is 206-150000 μg/ml for current ITIH-4 assay. The endogenous ITIH-4 in plasma samples is very high therefore instead of spiking the matrix to prepare QCs we prepared three different plasma dilutions as high, middle and low QCs at the following dilution ratios: non-diluted (HQC), 1:10 (MQC), and 1:80 (LQC). The matrix used for the QCs were pooled human plasma. All QCs were aliquoted in 50 μl, stored at −80° C. All QCs were thawed at room temperature and further diluted 1:1000 times using starting block buffer (PBS) before experiment (Final QC dilutions in the assay are 1:1000 (HQC), 1:10000 (MQC) and 1:80000 (LQC).

The acceptance criteria for precision experiments were defined as:
1. The mean value of the repeated samples should be within 20% of their respective nominal values except at LLOQ, where it should not deviate by more than 25%.
2. The precision determined at each concentration level should not exceed 20% of the CV except at LLOQ, where it should not exceed 25% of the CV.
3. Furthermore (Ref. 2) the total error should not exceed 30% (40% at LLOQ and ULOQ).

6 independent precision experiments were performed for inter-assay precision analysis. In each experiment, high, mid, and low levels of samples were repeated as triplets (N=3) for intra-assay precision analysis. % CV=(Standard deviation (SD)÷Average Measured concentration)×100. Each experiment was performed on a different day.

### Accuracy:

Accuracy is the closeness of the calculated mean of individual measurements to a nominal concentration. If the nominal concentration is the true concentration, then accuracy is expressed as trueness. The accuracy should be within 20% (25% at the LLOQ) of the nominal spiked concentration in at least 80% of the matrices evaluated (Ref. 2). In the current accuracy study, one high concentration (final concentration after spiking and dilution: 100000 pg/ml; 10% spike volume) and one low concentration (final concentration after spiking and dilution: 10000 pg/ml; 10% spike volume) of ITIH-4-1 protein were spiked into pooled plasma. % Recoveries were analyzed as well as the inter-assay parameters. Inter assay acceptance criteria is 20% for HQC and MQC and 25% for LQC (Ref. 2).

$$\% Recovery = ( Measured Conc . \div ( Spiked Conc . + Endogenous Conc . ) ) \times 100$$

### Experiments:

TABLE 17
Spike/recovery analysis (5 independent experiments).
Spiking volume was 10% of the final volume.

| | Experiment 1 | | | |
|---|---|---|---|---|
| Spike | Calc. | Calc. Cone. | | |
| (pg/mL) | Cone. | CV | Recovery (%) | |
| 100000 | 25959 | 6.9 | 100% | |
| 10000 | 13085 | 6.3 | 95% | |
| 0 | 12391 | 1.2 | | |
| | Experiment 2 | | | |
| Spike | | Calc. Cone. | | |
| (pg/mL) | Calc. Cone. | CV | Recovery (%) | |
| 100000 | 26050 | 0.0 | 101% | |
| 10000 | 13522 | 5.6 | 91% | |
| 0 | 13504 | 5.0 | | |
| | Experiment 3 | | | |
| Spike | | Calc. Cone. | | |
| (pg/mL) | Calc. Cone. | CV | Recovery (%) | |
| 100000 | 19319 | 9.2 | 92% | |
| 10000 | 9371 | 2.7 | 98% | |
| 0 | 8276 | 7.2 | | |
| | Experiment 4 | | | |
| Spike | | Calc. Cone. | | |
| (pg/mL) | Calc. Cone. | CV | Recovery (%) | |
| 100000 | 22567 | 2.1 | 101% | |
| 10000 | 9887 | 3.2 | 97% | |
| 0 | 8825 | 6.8 | | |
| | Experiment 5 | | | |
| Spike | Calc. | Calc. | Recovery | Inter-assay |
| (pg/mL) | Cone. | Cone. CV | (%) | % CV |
| 100000 | 23793 | 5.9 | 107% | 11% |
| 10000 | 10974 | 15.9 | 110% | 15% |
| 0 | 8466 | 3.9 | | 20% |

% Recovery = (Measured Conc. ÷ (Spiked Conc. + Endogenous Conc.)) × 100. Each experiment was performed on a different day.

### Parallelism:

Parallelism is an essential experiment characterizing relative accuracy for a ligand binding assay. By assessing the effects of dilution on the quantitation of endogenous analyte in matrix, selectivity, matrix effects, minimum required dilution, endogenous levels of healthy and diseased populations and the LLOQ are assessed in a single experiment. Dilution of samples should not affect the accuracy and precision. Two major factors that contribute to non-parallelism are: a difference between the immune-affinity characteristics of calibrator reference material and unknown analyte, to the capture and detection reagents; and matrix effects variances among calibration curve matrix, quality control matrix and study population matrix.

ITIH-4 endogenous concentrations are very high therefore plasma samples were not spiked but were diluted. In this experiment, 5 individual plasma samples were tested by making serial dilutions of the samples. Back-calculated concentrations of diluted samples are used to evaluate method parallelism. There are no clear requirements for parallelism acceptance criteria in biomarker assay development, but there is a general industry standard. Parallelism recovery must be less than or equal to 20% amongst the in-range measurements.

### Experiments:

TABLE 18
ITIH-4 Parallelism study: Parallelism in this project is a demonstration that the sample
dilution response curve is parallel to the standard concentration response curve.

Individual Plasma −113

| | | Calc. | Calc. | |
|---|---|---|---|---|
| Plasma | Raw | Cone. | Cone. | |
| Dilution | Signal | Mean | CV | Parallelism |
| 5000 | 23688 | 15823 | 3.6 | |
| 10000 | 10407 | 7452 | 2.9 | 94% |
| 20000 | 4803 | 3634 | 4.9 | 98% |
| 40000 | 2456 | 1923 | 3.4 | 106% |
| 80000 | 1182 | 930 | 0.4 | 97% |
| 160000 | 653 | 486 | 2.9 | 105% |

| | Individual Plasma −114 | | | | Individual Plasma −115 | | | |
|---|---|---|---|---|---|---|---|---|
| | | Calc. | Calc. | | | Calc. | Calc. | |
| Plasma | Raw | Cone. | Cone. | | Raw | Cone. | Cone. | |
| Dilution | Signal | Mean | CV | Parallelism | Signal | Mean | CV | Parallelism |
| 5000 | 24725 | 16523 | 3.2 | | 22060 | 14878 | 6.9 | |
| 10000 | 11687 | 8294 | 1.8 | 100% | 10422 | 7462 | 1.9 | 100% |
| 20000 | 5363 | 4029 | 1.1 | 97% | 4817 | 3644 | 1.6 | 98% |
| 40000 | 2871 | 2234 | 0.3 | 111% | 2567 | 2007 | 1.1 | 110% |
| 80000 | 1388 | 1095 | 0.1 | 98% | 1294 | 1020 | 3.5 | 102% |
| 160000 | 753 | 573 | 1.9 | 105% | 689 | 518 | 3.3 | 102% |
| | Individual Plasma −116 | | | | Individual Plasma −117 | | | |
| | | Calc. | Calc. | | | Calc. | Calc. | |
| Plasma | Raw | Cone | Cone | | Raw | Cone | Cone. | |
| Dilution | Signal | Mean | CV | Parallelism | Signal | Mean | CV | Parallelism |
| 5000 | 17533 | 12047 | 2.6 | | 15441 | 10718 | 2.4 | |
| 10000 | 8284 | 6035 | 1.3 | 100% | 7483 | 5493 | 2.7 | 102% |
| 20000 | 3780 | 2901 | 1.3 | 96% | 3532 | 2721 | 7.4 | 99% |
| 40000 | 2007 | 1581 | 3.9 | 109% | 1857 | 1465 | 1.3 | 108% |
| 80000 | 1112 | 372 | 2.2 | 110% | 1020 | 797 | 2.9 | 109% |
| 160000 | 593 | 434 | 0.4 | 100% | 577 | 420 | 0.9 | 106% |

Parallelism = (Measured Conc. of greater dilution × dilution factor/measured conc. of smaller dilution) × 100

### 1.1.2 Selectivity:

Selectivity is the ability of an assay to measure the analyte of interest in the presence of other constituents in the sample. A well-designed ligand binding assay should be able to accurately measure the analyte of interest from most of the study individuals without unique individual matrix biology interfering. In ITIH4 selectivity experiments 9 Individual plasma samples were spiked with two different concentrations of ITIH-4 and recovery percentages were analyzed. The accuracy should be within 20% (25% at the LLOQ) of the nominal spiked concentration in at least 80% of the matrices evaluated.

TABLE 19
ITIH-4 selectivity study: 10 individual human plasma were
spiked with high and low ITIH-4 protein concentration.
Individual Plasma −114

| | | Calc. | Calc. | |
|---|---|---|---|---|
| Spike | Raw | Cone. | Cone. | Recovery |
| (pg/mL) | Signal | Mean | CV | (%) |
| 100000 | 53356 | 23273 | 13.7 | 97% |
| 10000 | 24153 | 11097 | 0.2 | 95% |
| 0 | 22184 | 10249 | 5.6 | |

| Individual Plasma −115 | | | | | Individual Plasma −116 | | | |
|---|---|---|---|---|---|---|---|---|
| | | Calc. | Calc. | | | Calc. | Calc. | |
| Spike | Raw | Cone. | Cone. | Recovery | Raw | Cone. | Cone. | Recovery |
| (pg/mL) | Signal | Mean | CV | (%) | Signal | Mean | CV | (%) |
| 100000 | 54802 | 23870 | 1.9 | 103% | 52138 | 22780 | 4.4 | 107% |
| 10000 | 24745 | 11350 | 4.8 | 104% | 18595 | 8691 | 2A | 96% |
| 0 | 20185 | 9383 | 5.1 | | 16003 | 7552 | 7.7 | |
| Individual Plasma −117 | | | | | Individual Plasma −118 | | | |
| | | Calc. | Calc. | | | Calc. | Calc. | |
| Spike | Raw | Cone. | Cone. | Recovery | Raw | Cone. | Cone. | Recovery |
| (pg/mL) | Signal | Mean | CV | (%) | Signal | Mean | CV | (%) |
| 100000 | 52353 | 22865 | 10.9 | 104% | 51199 | 22395 | 7.5 | 104% |
| 10000 | 20959 | 9718 | 8.8 | 101% | 19145 | 5531 | 5.7 | 96% |
| 0 | 17372 | 8155 | 6.1 | | 16654 | 7840 | 4.0 | |
| Individual Plasma −119 | | | | | Individual Plasma −120 | | | |
| | | Calc. | Calc. | | | Calc. | Calc. | |
| Spike | Raw | Cone. | Cone. | Recovery | Raw | Cone. | Cone. | Recovery |
| (pg/mL) | Signal | Mean | CV | (%) | Signal | Mean | CV | (%) |
| 100000 | 72278 | 30951 | 4.1 | 106% | 62504 | 27001 | 6.8 | 109% |
| 10000 | 34178 | 15347 | 0.5 | 90% | 25019 | 11466 | 9.5 | 91% |
| 0 | 34514 | 15487 | 5.1 | | 24231 | 11130 | 0.2 | |
| Individual Plasma −121 | | | | | Individual Plasma −122 | | | |
| | | Calc. | Calc. | | | Calc. | Calc. | |
| Spike | Raw | Cone. | Cone. | Recovery | Raw | Cone. | Cone. | Recovery |
| (pg/mL) | Signal | Mean | CV | (%) | Signal | Mean | CV | (%) |
| 100000 | 60721 | 26277 | 7.9 | 108% | 59973 | 25976 | 0.9 | 116% |
| 10000 | 23957 | 11012 | 3.8 | 91% | 21935 | 10141 | 4.7 | 101% |
| 0 | 22928 | 10567 | 11.3 | | 18299 | 8562 | 1.8 | |

% Recovery = (Measured Conc. ÷ (Spiked Conc. + Endogenous Conc.)) × 100. Spiking volume was 10% of the final volume.

### Interference:

The endogenous matrix interferences can specifically or nonspecifically bind to capture/detection reagents or the analyte of interest and lead to an increase or decrease of the signal generated. For biomarker assays, specific matrix effects can be additionally caused by endogenous molecules with similar structure to the target analyte (e.g., homologous family members, isoforms and precursor proteins) or their natural ligands and the analogs of the ligands. Because the antibodies have been tested by the vendor on homologous protein from the same family for cross-reactivity, we focused on these four common interferences: hemolysates, lipids (triglyceride-rich lipoproteins), biotin and unconjugated bilirubin.

In this study, each substance was spiked at seven concentration levels in pooled human plasma. Spiking volume was 10% of the final volume.

### Experiments:

TABLE 20
Summary of accuracy obtained in interference study.

| | | Calc. Cone. | Calc. Cone. | |
|---|---|---|---|---|
| | Raw Signal | Mean | CV | % Bias |
| Hemolysates | | | | |
| (mg/dL) | | | | |
| 500 | 38959 | 11146 | 3.9 | 1% |
| 250 | 36554 | 10481 | 1.1 | −5% |
| 125 | 34552 | 9926 | 0.4 | −10% |
| 0 | 38444 | 11004 | 1.8 | |
| Lipids | | | | |
| (mg/dL) | | | | |
| 1000 | 49053 | 13933 | 1.3 | 19% |
| 500 | 42124 | 12021 | 2.4 | 3% |
| 250 | 43268 | 12336 | 3.2 | 6% |
| | 40912 | 11686 | 0.1 | |
| Bilirubin | | | | |
| (mg/dL) | | | | |
| 20 | 34476 | 9905 | 6.6 | −11% |
| 10 | 36008 | 10329 | 2.3 | −7% |
| 5 | 34300 | 9856 | 0.4 | −12% |
| 0 | 38993 | 11155 | 2.3 | |
| Biotin | | | | |
| (mg/mL) | | | | |
| 3 | 77161 | 21702 | 5.0 | 12% |
| 2 | 51758 | 14679 | 3.2 | 2% |
| 1 | 46275 | 13167 | 4.9 | −12% |
| 0 | 42128 | 12022 | 3.4 | |

### Summary:

1—The assay was not significantly affected by Hemolysate at concentrations up to 500 mg/dL (≤20% bias).
2—The assay was not significantly affected by Lipids at concentrations up to 1000 mg/dL (≤20% bias).
3—The assay was not significantly affected by unconjugated Bilirubin at concentrations up 20 mg/dL (≤20% bias).
4—The assay was not significantly affected by Biotin at concentrations up to 3 mg/mL (≤20% bias).

### Sample Stability: Freeze and Thaw and Short Term Stability

The sample stability under specific conditions for given time intervals was assessed. Stability evaluations (freeze-thaw and short term stability) covered the sample handling and storage conditions Stability samples were compared to freshly made calibrators and freshly diluted QCs. Conditions used in stability experiments reflects situations likely to be encountered during actual sample handling and analysis (e.g., long-term, room temperature storage; freeze-thaw cycles). Long term stability study will be done for both assay reagents and sample in −80° C. in the same test.

QC samples described above were used in this stability test. In each freeze and thaw cycle, HQC, MQC and LQC were taken out from −80° C. and thawed at room temperature for 30 minutes. The samples were then returned to −80° C. and kept for at least 24 hours before next freeze and thaw cycle.

The samples undergone various cycles of freeze and thaw were then assayed and compared with QC samples freshly taken out of −80° C.

In the short term stability test, HQC, MQC and LQC were taken out from −80° C. and incubated at 4° C. and room temperature for two hours, four hours and 24 hours. The samples were then assayed and compared with QC samples freshly taken out of −80° C.

The sample is accepted stable if the accuracy is within 20% (25% at the LLOQ) of the control samples (QC samples freshly taken out of −80° C.)

Method Validations. ELISA NCAM and ITIH4 detection and quantitation methods were validated by analyses of precision, inter-assay variation, accuracy, parallelism, and short-term stability. Precision analysis was performed to determine how well a method provides the same result when a single sample was tested repeatedly. Precision measures the random error of a method. Inter-assay variation analysis was performed to ensure repeatability and assay performance over time. Accuracy analysis was performed to investigate if the concentration-response relationship was similar in the calibration curve and the samples. Parallelism analysis was performed to demonstrate that a sample dilution response curve was parallel to the standard concentration response curve.

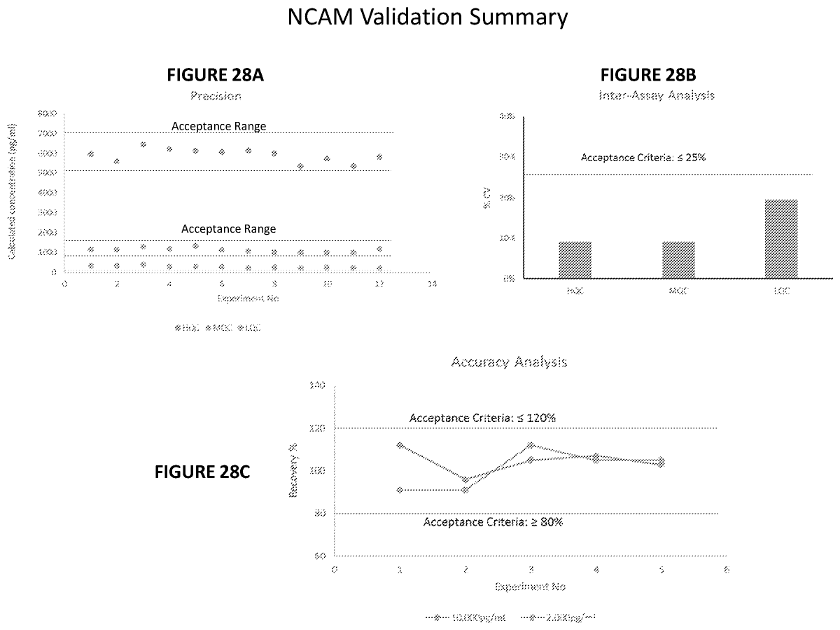

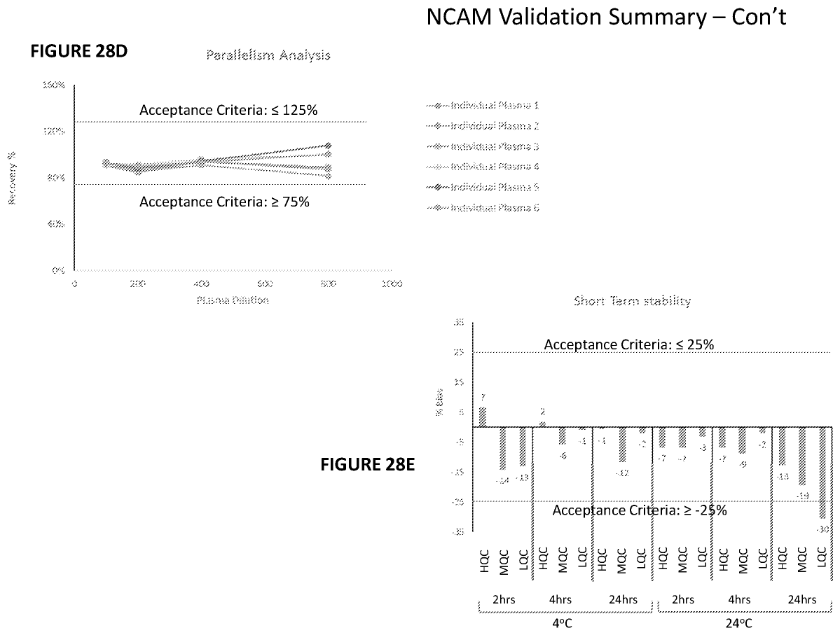

Results of the validation analyses of the NCAM detection and quantitation method are provided in FIGS. 28A-28E. Precision analyses were performed at high, medium and low concentrations (HQC, MQC, and LQC, respectively). For each concentration, twelve experiments were performed. Results indicated that all twelve experiments at the high, medium and low concentrations evaluated were within the acceptance range (see FIG. 28A). Inter-assay analyses of the results for the high, medium and low concentrations experiments were performed and results indicated that the coefficient of variation (% CV) of the data obtained for each group were within the acceptance criteria of less than or equal to 25% (see FIG. 28B). Accuracy analyses were performed at two concentrations of NCAM: 10,000 pg/mL and 2,000 pg/mL. Five experiments were performed at each concentration. Recovery percentages for all experiments at both concentrations (FIG. 28C) were within the acceptance criteria of greater than or equal to 80% and less than or equal to 120%. Parallelism analyses were performed using six individual plasma samples at four dilutions. Recovery percentages (FIG. 28D) were within the acceptance criteria of greater than or equal to 75% and less than or equal to 125% for all samples tested at each of the four dilutions. Short-term stability analyses were performed at two temperatures: 4° C. and 24° C. For each temperature, high, medium and low concentration samples (HQC, MQC, and LQC, respectively) were maintained at 2 hours, 4 hours, and 24 hours. Results show that all samples tested were within acceptance criteria of greater than or equal to −25% and less than or equal to 25% (see FIG. 28E).

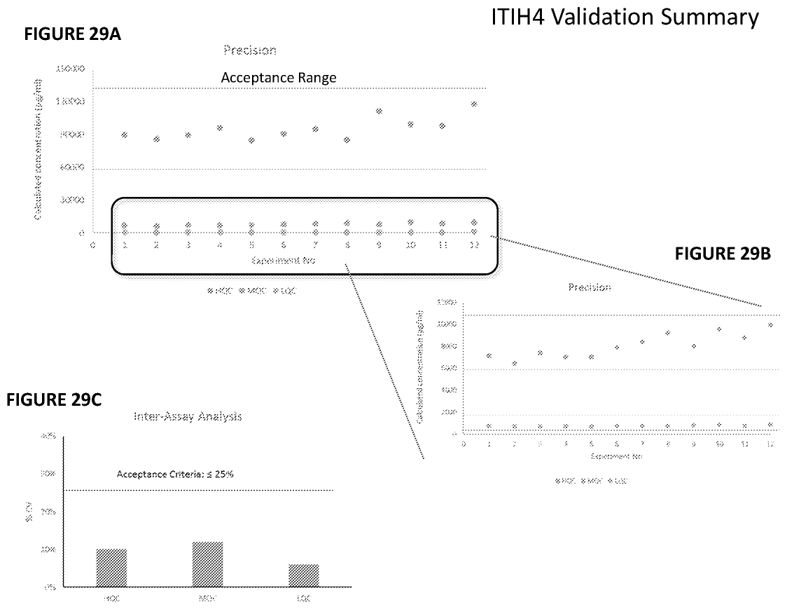

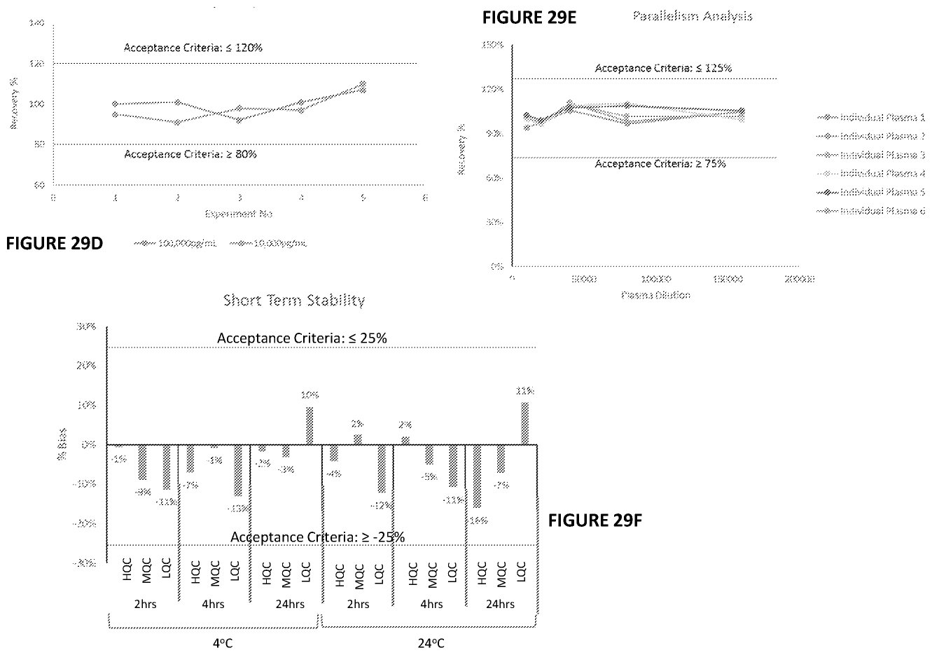

Results of the validation analyses for the ITIH4 detection and quantitation method are provided in FIGS. 29A-29F. Precision analyses were performed at high, medium and low concentrations (HQC, MQC, and LQC, respectively). For each concentration, twelve experiments were also performed. Results indicated that all twelve experiments at the high, medium and low concentrations evaluated were within the acceptance range (see FIGS. 29A-29B). Inter-assay analyses of the results for the high, medium and low concentrations experiments were performed and results indicated that the coefficient of variation (% CV) of the data obtained for each group were within the acceptance criteria of less than or equal to 25% (see FIG. 29C). Accuracy analyses were performed at two concentrations of ITIH4: 100,000 pg/mL and 10,000 pg/mL. Five experiments were performed at each concentration. Recovery percentages for all experiments at both concentrations were within the acceptance criteria of greater than or equal to 80% and less than or equal to 120% (see FIG. 29D). Parallelism analyses were performed using six individual plasma samples at five dilutions. Recovery percentages were within the acceptance criteria of greater than or equal to 75% and less than or equal to 125% for all samples tested at each of the dilutions (see FIG. 29E). Short-term stability analyses were performed at two temperatures: 4° C. and 24° C. For each temperature, high, medium and low concentration samples (HQC, MQC, and LQC, respectively) were maintained at 2 hours, 4 hours, and 24 hours. Results show that all samples tested were within acceptance criteria of greater than or equal to −25% and less than or equal to 25% (see FIG. 29F).

### Example 6: Identification of Biomarkers and Clinical Variables for Diagnosing Parkinson's Disease and for Stratifying Parkinson's Disease by Stage

Molecular diagnostics that identify and stratify Parkinson's Disease (PD) by stage are useful for determining the appropriate therapeutic intervention dose for a patient. Novel biomarkers and clinical variables that provide diagnostic utility in identifying PD, as well as stratifying it by stage, are described herein. The strategy for identification and evaluation of biomarkers included: (1) stratification of biomarkers along UPDRS and Hoehn & Yahr stages as described herein to identify predictive markers for the prodromal phase of disease; and (2) screening of multiple biofluids to overlay the pathophysiology of the disease.

Proteomics, metabolomics, lipidomics and genetic analysis were performed to identify biologic markers of PD for use in diagnostic testing and to identify and investigate correlations between biologic markers and clinical features of PD. The study design included single center observational study to assess biological markers in PD patients and healthy controls. The patient cohort included about 200 patients with diagnosis of PD and age/gender matched healthy individuals as controls, as summarized in Table 21, below.

TABLE 21
Patient Cohort

| Sample Counts | | Totals |
|---|---|---|
| PD | Male | 113 |
| | Female | 83 |
| Control | Male | 112 |
| | Female | 84 |

| | |
|---|---|
| Totals | 392 |

Patient biofluids including plasma and urine along with close to 1,000 clinical features were collected in the study. Table 22 below summarizes the procedures performed.

TABLE 22
Collection of Patient Characteristics and Clinical Features

| Procedure | Study Visit |
|---|---|
| Informed Consent | X |
| Demographics | X |
| Inclusion/Exclusion Criteria | X |
| Medical History | X |
| Family History | X |
| PD History | X |
| UK Parkinson's Disease Society Brain Bank | X |
| Clinical Diagnostic Criteria | |
| Concomitant Medications | X |
| Previous PD Medications | X |
| Vital Signs (Ht, Wt, BP, Pulse) | X |
| Montreal Cognitive Assessment (MoCA) | X^{C} |
| Hospital Anxiety and Depression Scale (HADS) | X |
| Parkinson's Disease Sleep Scale (PDSS-2) | X |
| Rapid eye movement sleep behavior | X |
| questionnaire (RBDQ1) | |
| Neurological Examination | X^{C} |
| UPDRS Parts I-IV | X^{C} |
| Modified Hoehn and Yahr | X^{C} |
| B-SIT (12-Item Smell Test) | X^{C} |
| Blood & Urine Specimen Collection | X |

CAll efforts were made to have assessments completed in the “ON” motor state

Plasma biomarkers for PD were evaluated individually and in combination with age and selected diagnostic utilities. The biomarkers evaluated in the analyses included: EMA/GA/MSA, NAP, NCAM and ITIH4. Diagnostic variables included in the analyses were performance on the smell test, anxiety test, and sleep test. Assessment of diagnostic value was performed for individual biomarkers, combinations of biomarkers alone, and combinations of biomarkers combined with age and clinical variables to identify optimal combinations.

The study included a PD patient cohort of 400, among which 199 were healthy and 201 had PD. CLIA tests were conducted for the six plasma biomarkers, in particular, EMA, GA, MSA, and NAP from a metabolomics study, as well as NCAM and ITIH4 from a proteomic study. NAP, MSA, GA, EMA, NCAM-1 and ITIH4 were detected and quantitated using methods of the invention described above.

Diagnostic utility variables identified from the clinical data file included the Smell test, as reflected in B-Sit score with variable named as “BSitTotal”, the Anxiety test, as reflected in HADs score with variable named as “HADsDTotal,” and the Sleep test, as reflected in the RBD indicator with variable named as “RBDNO.”

Table 23 provides a summary of data availability for each marker. Missing data points are indicated in the column “# of NA.” The number of data points above and below the quantile levels are indicated under “# of AQL” and “# of BQL”, respectively. Both AQL and BQL data points were treated as NA in the following analysis. Treating the single data point for each of EMA and NCAM that was above the quantile level (AQL) as NA (missing data) did not affect the analysis as there was only one data point for each EMA and NCAM. Regarding the 150 BQL data points for MSA, since the distribution of health to PD was 61 to 89, there was no significant trend indicating that one group was more likely to have BQL values and only slight differences in analytical results were detected between imputing BQL by lower bound and treating BQL as missing.

TABLE 23
Biomarker Data

| | # of NA | # of AQL | # of BQL |
|---|---|---|---|
| EMA | 0 | 1 | 0 |
| GA | 50 | 0 | 0 |
| MSA | 25 | 0 | 150 |
| NAP | 0 | 0 | 0 |
| NCAM | 0 | 1 | 0 |
| ITIH4 | 0 | 0 | 0 |

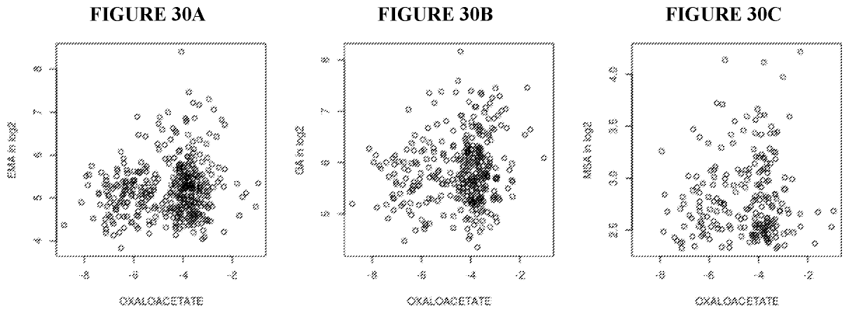

To evaluate the association between EMA/GA/MSA and oxaloacetate, normalized oxaloacetate values were used (raw and normalized data for oxaloacetate were highly correlated in terms of ordering (by spearman)). Results are shown in FIGS. 30A-30C. Scatterplots for EMA, GA, and MSA in log 2 versus normalized oxaloacetate (FIGS. 30A-30C) showed no particular pattern, e.g., linear association, between EMA, GA, or MSA and oxaloacetate. The weak associations between EMA, GA and MSA with oxaloacetate were confirmed by the corresponding correlation coefficients by spearman methods, which were 0.095, 0.124 and 0.014.

Diagnostic assessment of biomarkers was then performed for individual biomarkers and for combinations of biomarkers, as well as with age and diagnostic utility tests. Diagnostic assessment outputs included AUC value, ROC curve, sensitivity, PPV, NPV and OR (combined panel). Raw data, without normalization or imputation, were used for the analysis.

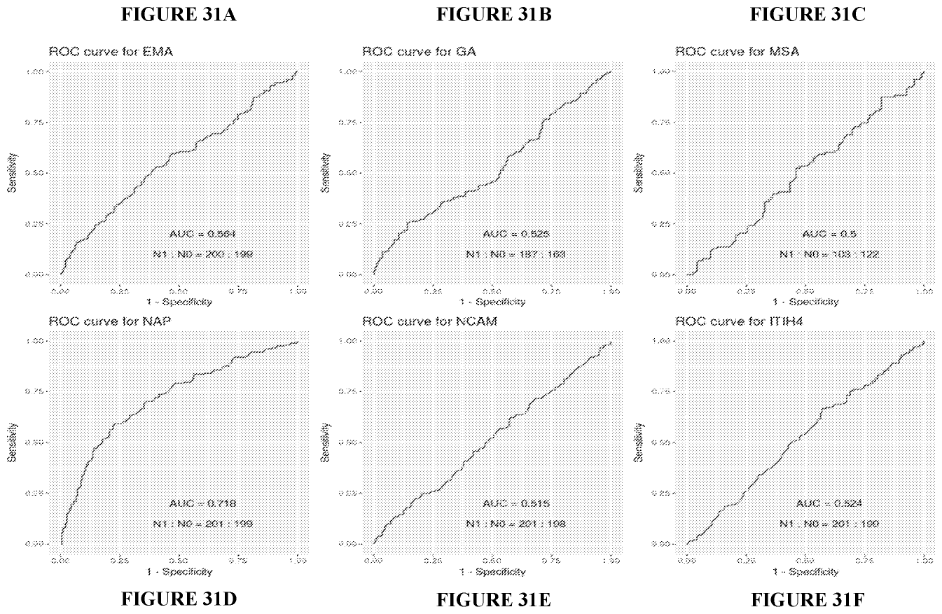

Biomarker Assessment. Individual biomarker assessment results are summarized in Table 24, which provides the individual AUC value for each of the six biomarkers, and in FIGS. 31A-31F, which provide the corresponding ROC curves.

TABLE 24
AUC Values for Biomarkers

| EMA | GA | MSA | NAP | NCAM | ITIH4 |
|---|---|---|---|---|---|
| 0.564 | 0.525 | 0.5 | 0.718 | 0.515 | 0.524 |

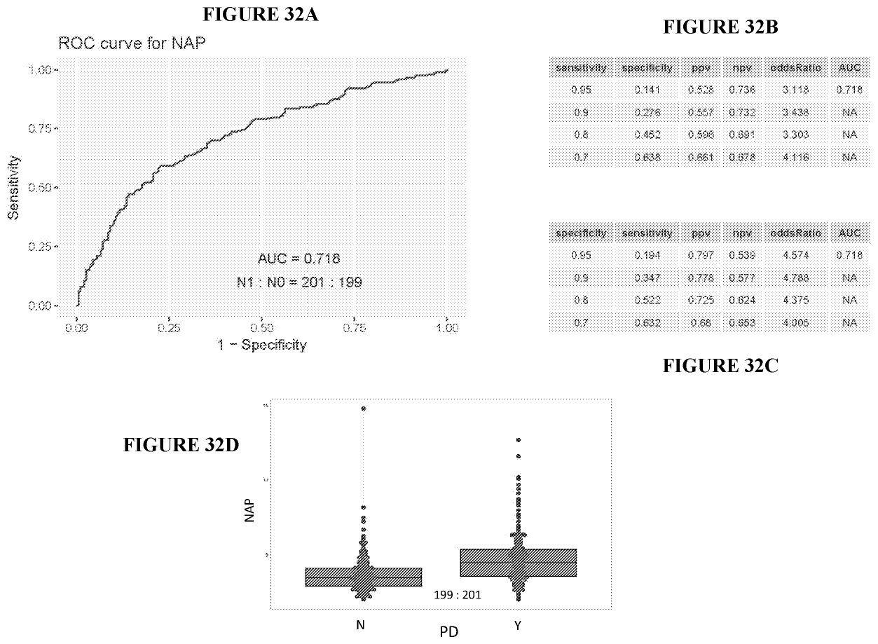

The individual biomarker assessment indicates that NAP has an AUC value of 0.718 and is a useful diagnostic marker for PD. Results of the diagnostic assessment for NAP are provided in FIGS. 32A-32D, which includes: ROC curve (FIG. 32A); values of sensitivity and specificity of 0.95, 0.9, 0.8 and 0.7, the corresponding specificity or sensitivity results, respectively, as well as PPV, NPV, oddsRatio and AUC results obtained (FIGS. 32B and 32C); and the Beeswarm plot of NAP vs. PD (FIG. 32D).

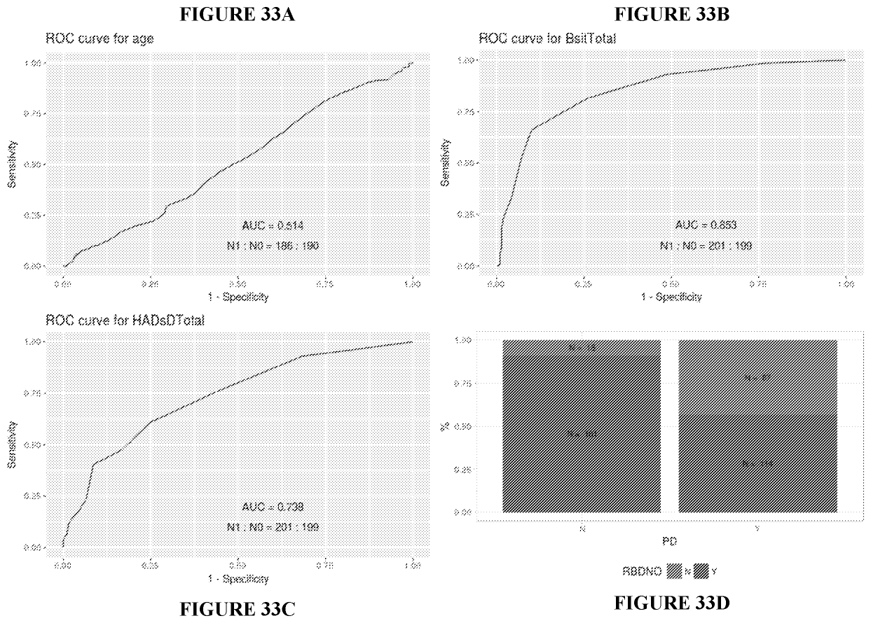

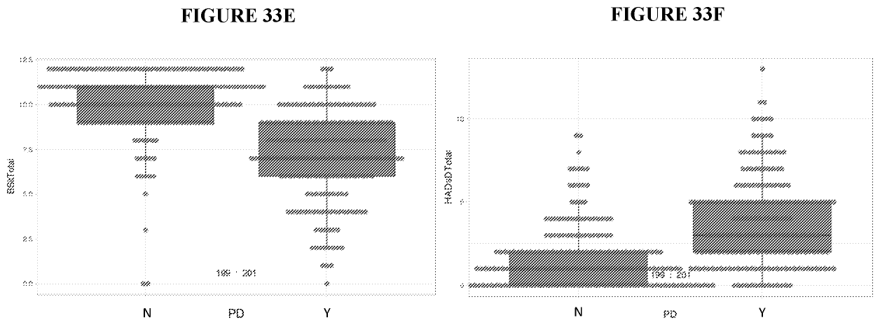

Clinical Variable Assessment. Diagnostic assessment of individual clinical variables were performed and results for age, performance on the smell test, anxiety test and sleep test are summarized in Table 20, as well as in FIGS. 33A-33F. Diagnostic assessment analysis utilized a complete set of data from the smell test, anxiety test and sleep test, and 24 fewer data points for analysis of the age variable.

For the smell test, a BSIT (Brief Smell Identification Test) score of 7 or greater is considered to indicate intact olfaction, while a BSIT score of 6 or lower is interpreted to mean impaired olfaction.

For the anxiety test, symptoms of anxiety and depression were assessed and a HADsDTotal (Hospital Anxiety and Depression Scale) test score of 7 or less indicates normal functioning, 8-10 indicates borderline abnormal results, and 11-21 indicates an abnormal case or the presence of anxiety and depression symptoms. For the sleep test, patients were evaluated for the presence or absence of REM sleep behavior disorder (RBD).

AUC values (Table 25) and ROC curves (FIGS. 33A-33C) are presented for age (FIG. 33A), smell test results (BsitTotal, FIG. 33B), and anxiety test results (HADsDTotal, FIG. 33C), as these are continuous variables. For the sleep test (RBDNO) (FIG. 33D), a p-value from Chi-square test and distribution plots is provided, as this is a binary variable. Among the clinical variables, all three diagnostic utility variables (smell, anxiety and sleep) were shown to distribute differently between PD and healthy patients, while age is distributed evenly between these two groups. FIGS. 33E-33F are the Beeswarm plots for the smell test (BSitTotal, FIG. 33E) and the anxiety test results (HADsDTotal, FIG. 33F).

TABLE 25
Individual assessment on clinical variables

| AUC | | | Chi-square p-value |
|---|---|---|---|
| age | BsitTotal | HADsDTotal | RBDNO |
| 0.514 | 0.853 | 0.738 | 1.749e−14 |

Combination Assessment. The diagnostic value of all possible combinations of biomarkers and clinical variables were then assessed, and a combination with a reasonably good AUC value, e.g., >about 0.8, was considered optimal.

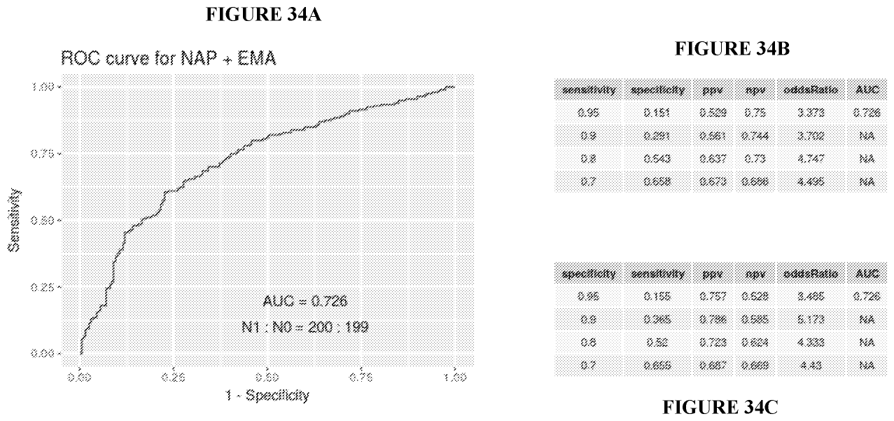

Results of various biomarker combination assessments indicated that the EMA+NAP combination was particularly useful based on an AUC value of 0.726. Diagnostic assessment results obtained for EMA and NAP are provided in FIGS. 34A-34C and include: ROC curve (FIG. 34A) and values of sensitivity and specificity of 0.95, 0.9, 0.8 and 0.7, corresponding statistics on sensitivity or specificity values, as well as PPV, NPV, OddsRatio, and AUC values obtained (FIGS. 34 B and 34C).

Results of various biomarker-clinical variable combinations assessments showed that six specific combinations of four variables have AUC values above 0.9. Two of the six combinations included NAP, BstiTotal and HADsTotal. In one of the two combinations, the fourth variable was age, and in the other of the two combinations, the fourth variable was RBDNO. The NAP-BstiTotal-HADsTotal-Age combination yielded a higher AUC value than the NAP-BstiTotal-HADsTotal-RBDNO combination. The NAP-BstiTotal-HADsTotal-Age combination also involved a smaller sample size due to 24 missing data points in age.

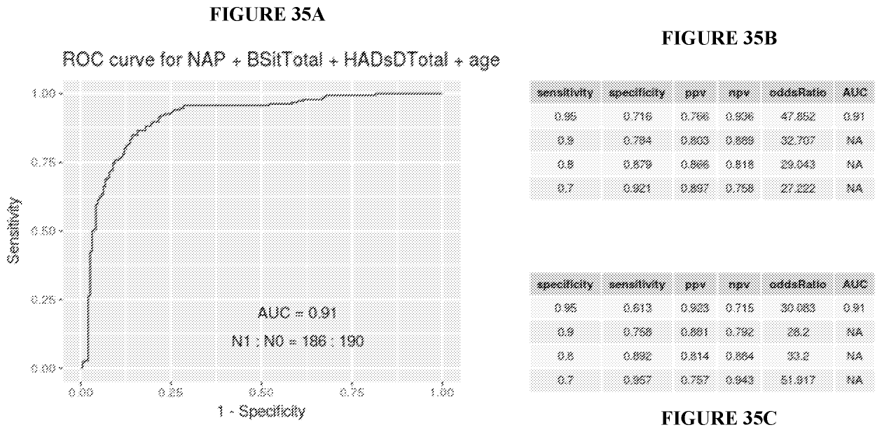

Diagnostic assessment results for these two biomarker-clinical variable combinations are provided in FIGS. 35A-35C. FIGS. 35A-35C provide diagnostic assessment results for the combination of NAP+BSitTotal+HADsDTotal+age and include: ROC curve (FIG. 35A), and sensitivity and specificity values of 0.95, 0.9, 0.8 and 0.7, the corresponding sensitivity or specificity values, respectively, as well as the PPV, NPV, OddsRatio, and AUC values obtained (FIG. 35B and FIG. 35C).

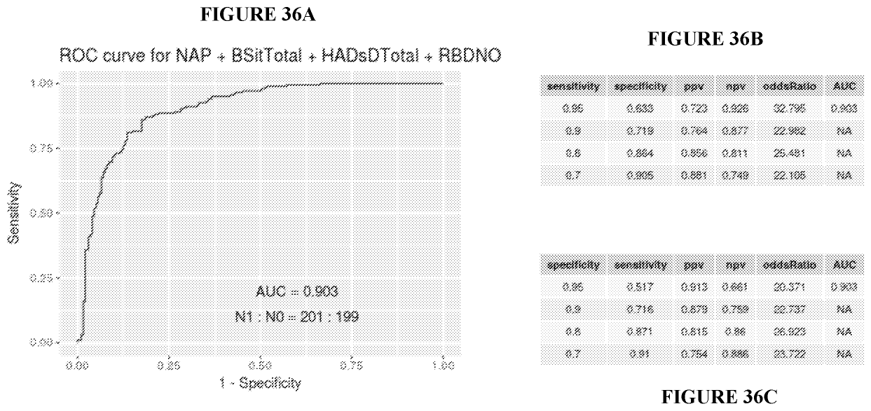

FIGS. 36A-36C provide diagnostic assessment results for the combination of NAP+BSitTotal+HADsDTotal+RBDNO and include: ROC curve (FIG. 36A), and sensitivity and specificity values of 0.95, 0.9, 0.8 and 0.7, the corresponding sensitivity or specificity values, respectively, as well as the PPV, NPV, OddsRatio, and AUC values obtained (FIG. 36B and FIG. 36C).

In sum, these studies indicate that EMA, GA and MSA did not correlate with oxaloacetate. The individual biomarker diagnostic assessment analyses indicated that the biomarker NAP has an AUC value of 0.718 and can be used for differentiating PD patients from healthy patients. Diagnostic assessment of individual clinical variables indicated that all three diagnostic utility variables were predictive for PD and healthy groups. Diagnostic assessments of combinations of biomarkers identified the EMA+NAP combination with an AUC value of 0.726 as particularly useful biomarker. Diagnostic assessments of biomarkers with clinical variables identified the NAP+BSitTotal+HADsDTotal+age combination and the NAP+BSitTotal+HADsDTotal+RBDNO combination as having AUC values larger than 0.9. The combination that included age was found to have a higher AUC value of 0.91 (based on a sample size with 24 fewer data points); while the combination that included RBDNA was found to have a lower AUC value of 0.903 (based on a full sample size).

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A method for diagnosing the presence of Parkinson's disease or the stage of Parkinson's disease in a subject, the method comprising:
(a) detecting the level of one or more Parkinson's Disease biomarkers in a biological sample from the subject, wherein the first marker is N-acetyl putrescine (NAP) or ethyl malonic acid (EMA); and
(b) comparing the level of the first marker in the biological sample with a first predetermined threshold value, wherein an increased or decreased level of the first marker as compared to the first predetermined threshold value indicates the presence of Parkinson's disease in the subject.

2. The method of claim 1, further comprising
detecting the level of a second marker in the biological sample from the subject, wherein the second marker is EMA or NAP, and wherein the first marker and the second marker are different markers; and
comparing the level of the second marker in the biological sample with a second predetermined threshold value, wherein an increased or decreased level of the second marker as compared to the second predetermined threshold value indicates the presence of Parkinson's disease in the subject.

3. The method of claim 1 or 2, further comprising determining the subject's performance on an anxiety test, a sleep test, a smell test, or any combination thereof, wherein an impaired performance on the anxiety test, the sleep test, the smell test, or any combination thereof indicates the presence of Parkinson's disease in the subject.

4. The method of claim 3, wherein impaired performance on the anxiety test is indicated where the anxiety test yields a HADsDTotal score of 8 or above.

5. The method of claim 3, wherein impaired performance on the sleep test is indicated where the sleep test indicates the presence of REM sleep behavior disorder.

6. The method of claim 3, wherein impaired performance on the smell test is indicated where the smell test yields a BsitTotal score of 6 or below.

7. The method of any one of the preceding claims, further comprising administering a treatment for Parkinson's disease where the diagnosis indicates the presence of Parkinson's disease in the subject.

8. The method of any one of the preceding claims, wherein the subject is suspected of having or being at risk of having Parkinson's disease.

9. The method of any one of the preceding claims, further comprising comparing the level of the first marker and/or the level of the second marker in the biological sample with that in a control sample,
wherein the biological sample is a sample obtained at a later time point than the control sample, the control sample being an earlier-in-time biological sample obtained from the subject, or
wherein the control sample is a Parkinson's disease-positive biological sample that is obtained from a subject with Parkinson's disease.

10. A method for monitoring Parkinson's disease in a subject, the method comprising:
(a) detecting the level of a first marker in a first biological sample obtained at a first time from a subject having Parkinson's disease, wherein the first marker is NAP or EMA;
(b) detecting the level of the first marker in a second biological sample obtained from the subject at a second time, wherein the second time is later in time than the first time; and
(c) comparing the level of the first marker in the first biological sample with that in the second biological sample, wherein a change in the level of the first marker indicates a change in Parkinson's disease status or stage in the subject.

11. The method of claim 10, further comprising detecting the level of a second marker in the first biological sample, wherein the second marker is EMA or NAP, and wherein the first marker and the second marker are different markers;
detecting the level of the second marker in the second biological sample; and
comparing the level of the second marker in the first biological sample with that in the second biological sample, wherein a change in the level of the second marker indicates a change in Parkinson's disease status or stage in the subject.

12. The method of claim 10 or 11, further comprising
determining the subject's performance on an anxiety test, a sleep test, a smell test, or any combination thereof at the first time point and at the second time point; and
comparing the subject's performance on the anxiety test, the sleep test, the smell test or any combination thereof at the first time point with that at the second time point;
wherein a change in performance on the anxiety test, the sleep test, the smell test, or any combination thereof between the first time point and the second time point indicates a change in Parkinson's disease status or stage in the subject.

13. The method of claim 12, wherein impaired performance on the anxiety test is indicated where the anxiety test yields a HADsDTotal score of 8 or above.

14. The method of claim 12, wherein impaired performance on the sleep test is indicated where the sleep test indicates the presence of REM sleep behavior disorder.

15. The method of claim 12, wherein impaired performance on the smell test is indicated where the smell test yields a BsitTotal score of 6 or below.

16. The method of any one of claims 10-15, wherein the subject is actively treated for Parkinson's disease prior to obtaining the second sample.

17. The method of any one of claims 10-15, wherein the subject is not actively treated for Parkinson's disease prior to obtaining the second sample.

18. The method of any one of claims 12-17, wherein a change in the level of the first marker, a change in the level of the second marker, a change in performance on the anxiety test, a change in performance on the sleep test, a change in performance on the smell test, or any combination thereof between the first time point and the second time point, indicates progression of Parkinson's disease in the subject.

19. The method of any one of claims 12-17, wherein a change or equivalent in the level of the first marker, a change or equivalent in the level of the second marker, a change or equivalent in performance on the anxiety test, a change or equivalent in performance on the sleep test, a change or equivalent in performance on the smell test, or any combination thereof between the first time point and the second time point, indicates progression of Parkinson's disease in the subject.

20. The method of any one of claims 12-19, further comprising comparing the level of the first marker in the first biological sample, the level of the first marker in the second biological sample, the level of the second marker in the first biological sample, the level of the second marker in the second biological sample, or any combination thereof, with that in a control sample, wherein the control sample is a sample from a Parkinson's disease-free subject, wherein a change in the level detected in the comparison indicates the presence of Parkinson's disease in the subject.

21. The method of any one of claims 12-19, further comprising comparing the level of the first marker in the first biological sample, the level of the first marker in the second biological sample, the level of the second marker in the first biological sample, the level of the second marker in the second biological sample, or any combination thereof, with that in a control sample, wherein the control sample is a sample from a subject with Parkinson's disease, wherein a change in the level detected in the comparison indicates a change in the stage of the Parkinson's disease in the subject.

22. The method of any one of the above claims 12-21, further comprising selecting a treatment for Parkinson's disease for the subject, and/or administering a treatment for Parkinson's disease to the subject, based on the status, the stage or the progression of the Parkinson's disease in the subject.

23. The method of any one of claims 12-21, further comprising administering a therapeutic for Parkinson's disease to the subject based on the status, the stage, or the progression of the Parkinson's disease in the subject.

24. The method of any one of claims 12-21, further comprising withholding and active treatment of the Parkinson's disease in the subject based on non-progression of the Parkinson's disease in the subject.

25. The method of any one of the preceding claims, wherein the stage of Parkinson's disease is based on the Hoehn-Yahr scale 0, scale 1, scale 1.5, scale 2, scale 2.5, scale 3, scale 4, or scale 5.

26. A method of treating Parkinson's disease in a subject comprising:
(a) obtaining a biological sample from the subject;
(b) submitting the biological sample to obtain diagnostic information as to the level of a first marker in the biological sample, wherein the first maker is NAP or EMA; and
(c) administering a therapeutically effective amount of a Parkinson's disease therapy to the subject if the level of the first marker obtained in (b) is above or below a first threshold level.

27. The method of claim 26, further comprising obtaining diagnostic information as to the level of a second marker in the biological sample, and administering a therapeutically effective amount of the Parkinson's disease therapy to the subject if the level of the second marker in the biological sample is above or below a second threshold level, wherein the second maker is EMA or NAP, and wherein the first maker and the second marker are different markers.

28. A method of treating Parkinson's disease in a subject comprising:
(a) obtaining diagnostic information as to the level of a first maker in a biological sample from the subject, wherein the first maker is NAP or EMA; and
(b) administering a therapeutically effective amount of a Parkinson's disease therapy to the subject if the level of the first marker obtained in (a) is above or below a first threshold level.

29. The method of claim 28, further comprising obtaining diagnostic information as to the level of a second marker in the biological sample, and administering a therapeutically effective amount of the Parkinson's disease therapy to the subject if the level of the second marker in the biological sample is above or below a second threshold level, wherein the second maker is EMA or NAP, and wherein the first maker and the second marker are different markers.

30. The method of any one of claims 26-29, further comprising obtaining the subject's performance on an anxiety test, a sleep test, a smell test, or any combination thereof, and administering a therapeutically effective amount of a Parkinson's disease therapy if the subject's performance on the anxiety test, the sleep test, the smell test, or any combination thereof is impaired.

31. The method of claim 30, wherein impaired performance on the anxiety test is indicated where the anxiety test yields a HADsDTotal score of 8 or above.

32. The method of claim 30, wherein impaired performance on the sleep test is indicated where the sleep test indicates the presence of REM sleep behavior disorder.

33. The method of claim 30, wherein impaired performance on the smell test is indicated where the smell test yields a BsitTotal score of 6 or below.

34. A method of treating Parkinson's disease in a subject suspected of having Parkinson's disease, the method comprising:
(a) obtaining a biological sample from the subject for use in identifying diagnostic information as to the level of a first marker in the biological sample, wherein the first maker is NAP or EMA;
(b) measuring the level of the first marker in the biological sample; and
(c) recommending to a healthcare provider to administer a Parkinson's disease therapy if the level of the first marker in the biological sample is above or below a threshold level.

35. The method of claim 34, further comprising measuring the level of a second marker in the biological sample, wherein the second maker is EMA or NAP, and wherein the first maker and the second marker are different markers; and recommending to a healthcare provider to administer a Parkinson's disease therapy if the level of the second marker in the biological sample is above or below a threshold level.

36. The method of claim 34 or 35, further comprising obtaining the subject's performance on an anxiety test, a sleep test, a smell test, or any combination thereof, and recommending to a health care provider to administer a Parkinson's disease therapy if the subject's performance on the anxiety test, the sleep test, the smell test, or any combination thereof is impaired.

37. The method of claim 36, wherein impaired performance on the anxiety test is indicated where the anxiety test yields a HADsDTotal score of 8 or above.

38. The method of claim 36, wherein impaired performance on the sleep test is indicated where the sleep test indicates the presence of REM sleep behavior disorder.

39. The method of claim 36, wherein impaired performance on the smell test is indicated where the smell test yields a BsitTotal score of 6 or below.

40. The method of any one of the preceding claims, wherein the level of the first marker and/or the level of the second marker are determined using immunoassay or ELISA.

41. The method of any one of the preceding claims, wherein the level of the first marker and/or the level of the second marker are determined using mass spectrometry.

42. The method of any one of the preceding claims, wherein the level of the first marker and/or the level of the second marker are determined using liquid chromatography with tandem mass spectrometry.

43. The method of any one of the preceding claims, wherein
the first maker is determined using a method comprising contacting the biological sample with a first reagent that selectively binds to the first marker to form a first marker-reagent complex and detecting the first maker-reagent complex; and/or
the second maker is determined using a method comprising contacting the biological sample with a second reagent that selectively binds to the second marker to form a second marker-reagent complex and detecting the second marker-reagent complex.

44. The method of any one of the preceding claims, wherein the biological sample comprises blood, serum, urine, cerebrospinal fluid, organ tissue, feces, skin, hair, or cheek tissue.

45. A kit for detecting Parkinson's disease in a subject, the kit comprising:
(a) a first reagent for measuring the level of a first marker in a biological sample from the subject, wherein the first maker is NAP or EMA;
(b) a second reagent for measuring the level of a second marker in the biological sample, wherein the second marker is EMA or NAP, and the first maker and the second marker are different markers; or
(c) a first reagent for measuring the level of a first marker in a biological sample from the subject, wherein the first maker is NAP or EMA, and a second reagent for measuring the level of a second marker in the biological sample, wherein the second marker is EMA or NAP, and the first maker and the second marker are different markers;
the kit further comprising a set of instructions for measuring the level of the first marker in the biological sample and/or the level of the second marker in the biological sample.

46. The kit of claim 45, further comprising test material for performing an anxiety test, a sleep test, a smell test, or any combination thereof.

47. The kit of claim 45 or 46, wherein the first reagent selectively binds to the first marker to form a first marker-reagent complex, and the second reagent selectively binds to the second marker to form a second marker-reagent complex.

48. The kit of claim 45 or 46, wherein the biological sample comprises blood, serum, urine, cerebrospinal fluid, organ tissue, feces, skin, hair, or cheek tissue.

49. A panel for use in (a) a method of detecting a first marker and/or a second maker for Parkinson's disease, (b) a method for determining the stage of Parkinson's disease in a subject, (c) a method of treating Parkinson's disease in a subject, or (d) a method of monitoring the treatment of Parkinson's disease in a subject, the panel comprising:
(i) a first reagent specific for detecting a first maker in a biological sample from the subject, and
(ii) a second reagent specific for detecting a second marker in the biological sample;
wherein the first maker is NAP or EMA, the second marker is EMA or NAP, and the first maker and the second marker are different markers.

50. The panel of claim 49, wherein the first reagent selectively binds to the first marker to form a first marker-reagent complex, and the second reagent selectively binds to the second marker to form a second marker-reagent complex.

51. The panel of claim 49 or 50, wherein the biological sample comprises blood, serum, urine, cerebrospinal fluid, organ tissue, feces, skin, hair, or cheek tissue.

52. A kit comprising the panel of any one of claims 49-51 and a set of instructions for obtaining diagnostic information as to the level of the first marker and the level of the second marker.

53. The kit of claim 52, further comprising test material for performing an anxiety test, a sleep test, a smell test, or any combination thereof.

54. Use of the panel of any one of claims 49-51 in a method for diagnosing and/or treating Parkinson's disease.
